SEQUENCE LISTING (45 sequences)

SEQ ID NO: 1 - DNA, 1186 bp, Musa acuminata
  CDS (55)...(1026)
tttggttgtgcctaacagagagagagagacagaccgatagcctcctcattcactatggcgatccgatcgccagcttcgctgctgttatttgcgttcctgatgcttgcgctcacgggaagactgcaggcccggcgcagctcatgcattggcgtctactggggacaaaacaccgacgagggaagcttagcagatgcttgtgccacaggcaactacgaatacgtgaacatcgccacccttttcaagtttggcatgggccaaactccagagatcaacctcgccggccactgtgaccctcggaacaacggctgcgcgcgcttgagcagcgaaatccagtcctgccaggagcgtggcgtcaaggtgatgctctccatcggaggtggcgggtcttatggcctgagttccaccgaagacgccaaggacgtagcgtcatacctctggcacagtttcttgggtggttctgctgctcgctactcgagacccctcggggatgcggttctggatggcatagacttcaacatcgccggagggagcacagaacactatgatgaacttgccgctttcctcaaggcctacaacgagcaggaggccggaacgaagaaagttcacttgagtgctcgtccgcagtgtcctttcccggattactggcttggcaacgcactcagaacagatctcttcgacttcgtgtgggtgcagttcttcaacaacccttcgtgccatttctcccagaacgctatcaatcttgcaaatgcgttcaacaattgggtcatgtccatccctgcgcaaaagctgttccttgggcttcctgctgctcctgaggctgctccaactggtggctacattccaccccatgatctcatatctaaagttcttccgatcctaaaggattccgacaagtacgcaggaatcatgctgtggactagataccacgacagaaactccggctacagttctcaagtcaagtcccacgtgtgtccagcgcgtcggttctccaacatcttatctatgccggtgaagtcttccaagtaaacctgaacggcgtagatgatcggtggtcgaaaactccgatcatcatgggtccccatccgtatccgtgcgttgctacgttatggtgtttcccttgtatgttggtcttttcaataatataataaggggttagttttacgtttccaaaaaaaaaaaaaaaaaa

SEQ ID NO: 2 - protein, 323 aa, Musa acuminata
MAIRSPASLLLFAFLMLALTGRLQARRSSCIGVYWGQNTDEGSLADACATGNYEYVNIATLFKFGMGQTPEINLAGHCDPRNNGCARLSSEIQSCQERGVKVMLSIGGGGSYGLSSTEDAKDVASYLWHSFLGGSAARYSRPLGDAVLDGIDFNIAGGSTEHYDELAAFLKAYNEQEAGTKKVHLSARPQCPFPDYWLGNALRTDLFDFVWVQFFNNPSCHFSQNAINLANAFNNWVMSIPAQKLFLGLPAAPEAAPTGGYIPPHDLISKVLPILKDSDKYAGIMLWTRYHDRNSGYSSQVKSHVCPARRFSNILSMPVKSSK

SEQ ID NO: 3 - protein, 90 aa, Musa acuminata
MAIRSPASLLLFAFLMLALTGRLQARRSSCIGVYWGQNTDEGSLSDKYAGIMLWTRYHDRNSGYSSQVKSHVCPARRFSNILSMPVKSSK

SEQ ID NO: 4 - protein, 67 aa, Musa acuminata
MEKCFNIIPSLLLISLLIKSSNAAGIAVYWGQNGNEGSLSPKYGGVMIWDRFNDAQSGYSNAIKGSV

SEQ ID NO: 5 - protein, 69 aa, Musa acuminata
MARTPQSTPLLISLSVLALIKTSYAGGIAIYWGQNGNEGTLSPKYGGVMIWSKFYDDQSGYSNSIKGSV

SEQ ID NO: 6 - protein, 73 aa, Musa acuminata
MTNMTLRKHVIYPLLFISCSLSKPSDASRGGIAIYWGQNGNEGNLSRKYGGVMIWSKFWDDKNGYSNSILASV

SEQ ID NO: 7 - protein, 64 aa, Musa acuminata
MIKYSPLLTASVSFLKALKLEAGDIVIYWGQNGNEGNLSPKYGGVMIWSKFYDNGYSNAILANV

SEQ ID NO: 8 - protein, 67 aa, Musa acuminata
MAAKIVSVLFLISSLIFASFESSHGGQIVIYWGQNGNEGNLSAKYGGVMIWSKAYDNGYSNAILASV

SEQ ID NO: 9 - DNA, 496 bp, Musa acuminata
  misc_feature 163, 387, 471: n = A,T,C or G
ggcacgagtacatcctctgcttcttcgagccttttcgccttccttcctcgtctaaccatgtcgacctgcggcaactgcgactgcgttgacaagagccagtgcgtgaagaagggaaacagctacggtatcgatattgttgagaccgagaagagctacgtcgacnaggtgatcgttgccgcagaagctgccgagcatgacggcaagtgcaagtgcggcgccgcctgcgcctgcaccgactgcaagtgtggcaactgagaagcacttgtgtcactaccactaaataaaagtttgcaatgcataaaaaacaaaagaacaaaaaaaaaaaaggaagaagaagaaggtgtggctatgtactctaataattcgggcaggctgataggttgtaanatgggataacgcagtatcatctgtgttatctctgtcctgtgtttacaactctcctatctatcctagtccatgaaatattattantattaaaaaaaaaaaaaaaaaaaaa

SEQ ID NO: 10 - DNA, 416 bp, Musa acuminata
ggcacgagggcacgaggttgcctctcgacatgtcgacctgcggcaactgcgactgcgctgacaagagccagtgcgtgaagaagggaaacagctacgctaccgagactgttgcgaccgagaagagcttcttggatggtgtagtcgatgccccagcagccgccgagacggagggagactgcaagtgtggtccttcctgcgcctgtgttgactgccaatgtggccagtgacagcttcttagctagtaatgacaatatataatatgttcgagtaaataacttggggcttgcatggctaatcgtttatcagtgtgtcatgatgtcagatgggatagggttgtgtctaccttgtctacatctgtactgttatcatacatgataaataaagaattattagtattaaaaaaaaaaaaaaaaaaa

SEQ ID NO: 11 - protein, 65 aa, Musa acuminata
MSTCGNCDCVDKSQCVKKGNSYGIDIVETEKSYVDEVIVAAEAAEHDGKCKCGAACACTDCKCGN

SEQ ID NO: 12 - protein, 67 aa, Musa acuminata
MSTCGNCDCADKSQCVKKGNSYATETVATEKSFLDGVVDAPAAAETEGDCKCGPSCACVDCKQCGNQ

SEQ ID NO: 13 - protein, 63 aa, Musa acuminata
MSDKCGNCDCADSSQCVKKGNSTETVATDKSFIEDVVMGVPAAESGGDCKCGTSCPCVNCTCD

SEQ ID NO: 14 - protein, 66 aa, Musa acuminata
MSGKCDNCDCADSTQCVKKGNSYDLVTVATDNRSMETVFMDVPAAESGGDCKCGTGCSCVSCTCDH

SEQ ID NO: 15 - protein, 65 aa, Musa acuminata
MSDKCDNCDCADSTQCVKKGSSYTAVTIATDNRIMTVVMDVPAAENGGDCKCGPSCSCVNCTCDH

SEQ ID NO: 16 - DNA, 1423 bp, Musa acuminata
taagcttccgtgccaaagcggtctgcctttctacgccgcatcgggaaggggaaacacaaaaaaaagatcaggaagatgatgctgacacgagaggtggaaggaagtttaccgctctcccataatagagattcctttggatgcttctcccggtgggtgtgtggagcacagacactgaatgtggtccgtcgttccaatccctcacgtaatcgggccgtctccggctataaataaccccccccgaccgagcgaacgcttctaaccaggaacgcataccacaccacaatttgttgagccgttgtgcttgttgcctctcgacatgtcgacctgcggcaactgcgactgcgctgacaagagccagtgcgtgtaagttctcttcctccccgccctcccacctctttgtgatacacacaacaaatatgcatgagggttgagtttaatattgaccacaagaacttgggtttgctcctgcaggaagaagggaaacagctacgctaccgagactgttgcgaccgagaagaggtattattgatctctctcatgggtgagggtgtgggagtatcttgtccgcatgatgaaattccacaacatgatgactcagcaaacaagatccttttattcttgagaaaacaactaaaagaagaaaaaaaaaacagagaatatatctgcgattatttctttttgagtgatgtggaattccatgccatagcttaaaactattttcgaagtcgaagcatattacatacctcttgatgaattagtaaggatgattaaaagtaagccatctaaagcagagtaactacttacgtttctttcatgtcatctctgtcttacagcttcttggatggtgtagtcgatgccccagcagccgccgaaacggagggagactgcaagtgtggtccttcctgcgcctgtgttgactgccaatgtggccagtgacagcttcttagctagtaatgacaatatataatatgttcgagtaaataacttggggcttgcatggctaatcgtttatcagtgtgtcatgatgtcagatgggatagggttgtgtctaccttgtctacatctgtactgttatcatacatgataaataaagaattattagtattaatttggtttcaggtgataactactgctcctttcaaccgaatcactactgttacgtgaacaaacatgtaatagtagtgattcagtaggacgacttttgtctatttaacttttgctttgggttgcaaaaatatgttcttcctgattcacgaaagagggtgtccatgagcattcggctattgagcgatgttggatgaggcctcaaagggaagaatttatgcttaggactctgagttcgatggttgccaccgacctcctcaagtaccaagacacatacccttccttccgaggcctatccaacatcgctcgtatcgtcgac

SEQ ID NO: 17 - DNA, 3559 bp, Musa acuminata
  misc_feature (1)...(3559): Complement
attggacccacgcggtggcggccgctctagaatagtggatcccccgggctgcaggaattctaaaatctattcttttttattttattaattaaattaaattaattttttattgtttggtatttagcctaacattcccggactcctctatttttggagattgaatacaaaattcttctcccatctaaagttattttaattttgaagatcatatggctgacatataaagcaaatatgtcaaaggtagttttcaccgtccacacgatagaaacaacaaagtagggtaattaaatttgttccgtcatcacaaagcacaacaccaaaatattcacttaatcaaatcctcactataaataataatccttcaaactgcaactctaaacaatgaggttctctctcccagcaacgttcttttctgaacacaaagatttgccacaaccttagctgacttttaatatcagtggtctctggacaagattcttgttgcacgctaaaattcgaactaaaatcagatcgagttatatccgtaattgagattgatgaccgaaccgattttaagagtactctccgtaacttgggattaataaaattaataaggtaggtatcagttattttagatgataaaaatcttgatagtttgaatctcatcttagtcacttatttttaattaaaaataataataataatttgattaatctgattggaaaaaaaaaaagttctctagccattaaagtctggtaggacatagaaattaatgaattaaactgtaaccataaggttgaatttttgaacacatgtacaggaaaattgatttgttgaagtcatgtctaatcaatgcagcagtttacagcttggtgtgacttccacaactataggcttatcccctgggagtcgaggatcaaacgtgtgagcaatattctcccttcctgatgataaactatgatggctgttaggtgtgtaagcactccaaattttccatcaatgtggaattggaagagttcacgcactgacggaccaactcggtttgttcagtctggtgactactgctgagcatgagaaaatggttgatggtagcaagttgcaaatgtacctgacctcatcttaaagactgttgattagatgcatgcattgattacgtctcttccatctttaactcttttgatcgatgcatcgtcttaattaggtcaaggacatgtgatgacaagaatctattccactatttgtgacccatattccaaatggaacaagacttccaagtcctcatccagaattttggaagggataaggatggtggggagaaagaacaagctgttgcctttcgttttcttctatcaggaagccaagagtttcaagaggagggtagacctgaggggatgatgcctgtgtcgaaacctctatataaggagtaggaacacagcatgttgatgaacacaaaccatttcagcggggaagaagagaacccttttgacagagttgttgtcatggcaacaaaagcttctctctccataaaaggctttgccttgctggtttcagtccttgtagcagttccaacaagttctctctctctctctctctctctctctctctctctctctctctctctctcatattatacatttgattgttagctcttacaaatttattagggtttttataagagttcaagcttttggtaatttaatcatggtaggttatattttcaaaacttgtaacctgcattttgtctctttatttcatgcaatattcttttccttgattggcttacgtcatttacttgagttagctcatatgtaactgtttaaatatttgggattattggttaacggataaaaaaaattaattgattttagatacaatgctatatatatatatatatatatatatatatatatatatatatatatatatatatattataggtagaaacttggtataattcacacgtatgttcgctttatctgaataaaatgagtagtcctttcaatgcagattagtcttactccacttgcagatgcacgaccaatttgcttgatcatcttccatagagcaccacagctaagtctccgatgtgttctactgcaggagtgcaatcgattggtgtctgctacggaatgctcggcaacaatcttcccccgcccagcgaggtggtcagtctctacaaatccaacaacatcgcgaggatgagactctacgatccaaaccaggccgccctgcaagccctcaggaactccaacatccaagtcctgttggatgtcccccgatccgacgtgcagtcactggcctccaatccttcggccgccggcgactggatccggaggaacgtcgtcgcctactggcccagcgtctcctttcgatacatagctgtcggaaacgagctgatccccggatcggatctggcgcagtacatcctccccgccatgcgcaacatctacaatgctttgtcctcggctggcctgcaaaaccagatcaaggtctcgaccgcggtcgacacgggcgtcctcggcacgtcctaccctccctccgccggcgccttctcctccgccgcccaggcgtacctgagccccatcgtgcagttcttggcgagtaacggagcgccgctcctggtcaatgtgtacccttattttagctacaccggcaacccgggacagatctcgctgccctacgccctgttcacggcctccggcgtcgtcgtgcaggatgggcgattcagctatcagaacctgttcgacgccatcgtcgacgcggtcttcgcggcgctggagagagtgggaggggcgaacgtggcggtggtggtgtcggagagcgggtggccgtcggcgggcggaggagccgaagcgagcaccagcaacgcgcagacgtacaaccagaacttgatcaggcatgttggcggaggaacgccgaggagaccagggaaggagatcgaggcatacatattcgagatgttcaacgagaaccagaaggctggagggatcgagcagaactttggcctgttttatcccaacaagcagcccgtataccaaataagcttttagaaactaacttgtaaggttgatgaatcatctcctacctacctacctacctacgaataaaacatgaaataaagcaccaaaataaagggagaattctgatcttggagaaagttgaatcatgatgatatataacaaacacccctctttactcattatcagtatgttacaagtttcttgaaacttgaacggatcacaatttggacctacaagtattttgggtcataattatttcattgaactatatattcaaaaaaagatgtgtttggagtgcttaatacagtatgacttcagtttgcaagattacctcttcagcgtcagcttcagcatgccaaaaaaccatcatctgctatggggcatgttttacaccttgatggtgctacatcatcatcattcatgtttcattttaggtctcgtgctctttatatagatcacataaaagtttggatcgcttcaagtttctaggttacattgtatgcagcactttgagcctactgaacattgtgactgccttttagaacattggactgcaggaa

SEQ ID NO: 18 - DNA, 3559 bp, Musa acuminata
taacctgggtgcgccaccgccggcgagatcttatcacctagggggcccgacgtccttaagattttagataagaaaaaataaaataattaatttaatttaattaaaaaataacaaaccataaatcggattgtaagggcctgaggagataaaaacctctaacttatgttttaagaagagggtagatttcaataaaattaaaacttctagtataccgactgtatatttcgtttatacagtttccatcaaaagtggcaggtgtgctatctttgttgtttcatcccattaatttaaacaaggcagtagtgtttcgtgttgtggttttataagtgaattagtttaggagtgatatttattattaggaagtttgacgttgagatttgttactccaagagagagggtcgttgcaagaaaagacttgtgtttctaaacggtgttggaatcgactgaaaattatagtcaccagagacctgttctaagaacaacgtgcgattttaagcttgattttagtctagctcaatataggcattaactctaactactggcttggctaaaattctcatgagaggcattgaaccctaattattttaattattccatccatagtcaataaaatctactatttttagaactatcaaacttagagtagaatcagtgaataaaaattaatttttattattattattaaactaattagactaacctttttttttttcaagagatcggtaatttcagaccatcctgtatctttaattacttaatttgacattggtattccaacttaaaaacttgtgtacatgtccttttaactaaacaacttcagtacagattagttacgtcgtcaaatgtcgaaccacactgaaggtgttgatatccgaataggggaccctcagctcctagtttgcacactcgttataagagggaaggactactatttgatactaccgacaatccacacattcgtgaggtttaaaaggtagttacaccttaaccttctcaagtgcgtgactgcctggttgagccaaacaagtcagaccactgatgacgactcgtactcttttaccaactaccatcgttcaacgtttacatggactggagtagaatttctgacaactaatctacgtacgtaactaatgcagagaaggtagaaattgagaaaactagctacgtagcagaattaatccagttcctgtacactactgttcttagataaggtgataaacactgggtataaggtttaccttgttctgaaggttcaggagtaggtcttaaaaccttccctattcctaccacccctctttcttgttcgacaacggaaagcaaaagaagatagtccttcggttctcaaagttctcctcccatctggactcccctactacggacacagctttggagatatattcctcatccttgtgtcgtacaactacttgtgtttggtaaagtcgccccttcttctcttgggaaaactgtctcaacaacagtaccgttgttttcgaagagagaggtattttccgaaacggaacgaccaaagtcaggaacatcgtcaaggttgttcaagagagagagagagagagagagagagagagagagagagagagagagagagtataatatgtaaactaacaatcgagaatgtttaaataatcccaaaaatattctcaagttcgaaaaccattaaattagtaccatccaatataaaagttttgaacattggacgtaaaacagagaaataaagtacgttataagaaaaggaactaaccgaatgcagtaaatgaactcaatcgagtatacattgacaaatttataaaccctaataaccaattgcctattttttttaattaactaaaatctatgttacgatatatatatatatatatatatatatatatatatatatatatatatatatatataatatccatctttgaaccatattaagtgtgcatacaagcgaaatagacttattttactcatcaggaaagttacgtctaatcagaatgaggtgaacgtctacgtgctggttaaacgaactagtagaaggtatctcgtggtgtcgattcagaggctacacaagatgacgtcctcacgttagctaaccacagacgatgccttacgagccgttgttagaagggggcgggtcgctccaccagtcagagatgtttaggttgttgtagcgctcctactctgagatgctaggtttggtccggcgggacgttcgggagtccttgaggttgtaggttcaggacaacctacagggggctaggctgcacgtcagtgaccggaggttaggaagccggcggccgctgacctaggcctccttgcagcagcggatgaccgggtcgcagaggaaagctatgtatcgacagcctttgctcgactaggggcctagcctagaccgcgtcatgtaggaggggcggtacgcgttgtagatgttacgaaacaggagccgaccggacgttttggtctagttccagagctggcgccagctgtgcccgcaggagccgtgcaggatgggagggaggcggccgcggaagaggaggcggcgggtccgcatggactcggggtagcacgtcaagaaccgctcattgcctcgcggcgaggaccagttacacatgggaataaaatcgatgtggccgttgggccctgtctagagcgacgggatgcgggacaagtgccggaggccgcagcagcacgtcctacccgctaagtcgatagtcttggacaagctgcggtagcagctgcgccagaagcgccgcgacctctctcaccctccccgcttgcaccgccaccaccacagcctctcgcccaccggcagccgcccgcctcctcggcttcgctcgtggtcgttgcgcgtctgcatgttggtcttgaactagtccgtacaaccgcctccttgcggctcctctggtcccttcctctagctccgtatgtataagctctacaagttgctcttggtcttccgacctccctagctcgtcttgaaaccggacaaaatagggttgttcgtcgggcatatggtttattcgaaaatctttgattgaacattccaactacttagtagaggatggatggatggatggatgcttattttgtactttatttcgtggttttatttccctcttaagactagaacctctttcaacttagtactactatatattgtttgtggggagaaatgagtaatagtcatacaatgttcaaagaactttgaacttgcctagtgttaaacctggatgttcataaaacccagtattaataaagtaacttgatatataagtttttttctacacaaacctcacgaattatgtcatactgaagtcaaacgttctaatggagaagtcgcagtcgaagtcgtacggttttttggtagtagacgataccccgtacaaaatgtggaactaccacgatgtagtagtagtaagtacaaagtaaaatccagagcacgagaaatatatctagtgtattttcaaacctagcgaagttcaaagatccaatgtaacatacgtcgtgaaactcggatgacttgtaacactgacggaaaatcttgtaacctgacgtcctt

SEQ ID NO: 19 - protein, 1131 aa, Musa acuminata
IGPTRWRPLNSGSPGLQEFNLFFFILLIKLNFFIVWYLAHSRTPLFLEIEYKILLPSKVILILKIIWLTYKANMSKVVFTVHTIETTKGNICSVITKHNTKIFTSNPHYKSFKLQLTMRFSLPATFFSEHKDLPQPLTFNISGLWTRFLLHAKIRTKIRSSYIRNDPNRFEYSPLGINKINKVGISYFRKSFESHLSHLFLIKNNNNNLINLIGKKKSSLAIKVWDIEINELNCNHKVEFLNTCTGKLICSHVSMQQFTAWCDFHNYRLIPWESRIKRVSNILPSTMMAVRCVSTPNFPSMWNWKSSRTDGPTRFVQSGDYCAENGWQVANVPDLILKTVDMHALITSLPSLTLLIDASSLGQGHVMTRIYSTICDPYSKWNKTSKSSSRILEGIRMVGRKNKLLPFVFFYQEAKSFKRRVDLRGCLCRNLYIRSRNTACTQTISAGKKRTLLTELLSWQQKLLSPKALPCWFQSLQFQQVLSLSLSLSLSLSLSLSHIIHLIVSSYKFIRVFIRVQAFGNLIMVGYIFKTCNLHFVSLFHAIFFSLIGLRHLLELAHMLFKYLGLLVNGKKLIDFRYNAIYIYIYIYIYIYIYIYIYYRKLGIIHTYVRFIIKVVLSMQISLTPLADARPICLIIFHRAPQLSLRCVLLQECNRLVSATECSATIFPRPARWSVSTNPTTSRGDSTIQTRPPCKPSGTPTSKSCWMSPDPTCSHWPPILRPPATGSGGTSSPTGPASPFDTLSETSSPDRIWRSTSSPPCATSTMLCPRLACKTRSRSRPRSTRASSARPTLPPPAPSPPPPRRTAPSCSSWRVTERRSWSMCTLILATPATRDRSRCPTPCSRPPASSCRMGDSAIRTCSTPSSTRSSRRWREWEGRTWRWWCRRAGGRRRAEEPKRAPATRRRTTRTSGMLAEERRGDQGRRSRHTYSRCSTRTRRLEGSSRTLACFIPTSSPYTKAFRNLVRLMNHLLPTYLPTNKTNKAPKRENSDLGESIMMIYNKHPSLLIISMLQVSNLNGSQFGPTSILGHNYFIELYIQKKMCLECLIQYDFSLQDYLFSVSFSMPKNHHLLWGMFYTLMVLHHHHSCFILGLVLFIITKFGSLQVSRLHCMQHFEPTEHCDCLLEHWTAG

SEQ ID NO: 20 - protein, 1126 aa, Musa acuminata
LDPRGGGRSRIVDPPGCRNSKIYSFLFYLNNFLLFGIPNIPGLLYFWRLNTKFFSHLKLFFRSYGHIKQICQRFSPSTRKQQSRVIKFVPSSQSTTPKYSLNQILTINNNPSNCNSKQGSLSQQRSFLNTKICHNLSLLISVVSGQDSCCTLKFELKSDRVISVIEIDDRTDFKSTLRNLGLIKLIRVSVILDDKNLDSLNLILVTYFLKIIIIILILEKKKVLPLKSGRTKLMNTVTIRLNFTHVQENFVEVMSNQCSSLQLGVTSTTIGLSPGSRGSNVAIFSLPDDKLWLLGVALQIFHQCGIGRVHALTDQLGLFSLVTTAEHEKMVDGSKLQMYLTSSRLLIRCMHLRLFHLLFSMHRLNVKDMQESIPLFVTHIPNGTRLPSPHPEFWKGGWWGERTSCCLSFSSIRKPRVSRGGTGDDACVETSIGVGTQHVDEHKPFQRGRREPFQSCCHGNKSFSLHKRLCLAGFSPCSSSNKFSLSLSLSLSLSLSLSLILYILLALTNLLGFLEFKLLVISWVIFSKLVTCILSLYFMQYSFPLAYVIYLSLICNCLNIWDYWLTDKKNLILDTMLYIYIYIYIYIYIYIYIYIIIGRNLVFTRMFALSENESFQCRLVLLHLQMHDQFASSSIEHHSVSDVFYCRSAIDWCLLRNARQQSSPAQRGGQSLQIQQHREDETLRSKPGRPASPQELQHPSPVGCPPIRRAVTGLQSFGRRRLDPEERRRLLAQRLLSIHSCRKRADPRIGSGAVHPPRHAQHLQCFVLGWPAKPDQGLDRGRHGRPRHVLPSLRRRLLLRRPGVPEPHRAVLGERSAAPGQCVPLFLHRQPGTDLAALRPVHGLRRRRAGWAIQLSEPVRRHRRRGLRGAGESGRGERGGGGVGERVAVGGRRSRSEHQQRADVQPELDQACWRRNAEETREGDRGIHIRDVQREPEGWRDRAELWPVLSQQAARIPNLLETNLGIISYLPTYLRIKHEIKHQNKGRILILEKVESYITNTPLYSLSVCYKFLETTDHNLDLQVFWVIIISLNYIFKKRCVWSAYSMTSVCKITSSASASACQKTIICYGACFTPCYIIIIHVSFVSCSLYRSHKSLDRFKFLGYIVCSTLSLLNIVTAFNIGLQE

SEQ ID NO: 21 - protein, 1121 aa, Musa acuminata
NWTHAVAAALEWIPRAAGILKSILFYFINIKIFYCLVFSLTFPDSSIFGDIQNSSPISYFNFEDHMADISKYVKGSFHRPHDRNNKVGLNLFRHHKAQHQNIHLIKSSLIIILQTATLNNEVLSPSNVLFTQRFATTLADFYQWSLDKILVARNSNNQIELYPLRLMTEPILRVLSVTWDNGRYQLFMIKILIVISSSLIFNKFDSDWKKKKFSSHSLVGHRNIKLPGIFEHMYRKIDLLKSCLINAAVYSLVLPQLAYPLGVEDQTCEQYSPFLMINYDGCVCKHSKFSINVELEEFTHRTNSVCSVWLLLSMRKWLMVASCKCTPHLKDCLDACIDYVSSIFNSFDRCIVLIRSRTCDDKNLFHYLPIFQMEQDFQVLIQNFGRDKDGGEKEQAVAFRFLLSGSQEFQEEGRPEGMMPVSKPLYKEEHSMLMNTNHFSGEEENPFDRVVVMATKASLSIKGFALLVSVLVAVPTSSLSLSLSLSLSLSLSLSYYTFDCLLQIYGFYKSSSFWFNHGRLYFQNLPAFCLFISCNILFLDWLTSFTVSSYVTVIFGIIGRIKKINFIQCYIYIYIYIYIYIYIYIYILVETWYNSHVCSLYNKMSSPFNADSYSTCRCTTNLLDHLPSTTAKSPMCSTAGVQSIGVCYGMLGNNLPPPSEVVSLYKSNNIARMRLYDPNQAALQALRNSNIQVLLDVPRSDVQSLASNPSAAGDWIRRNVVAYWPSVSFRYIAVGNELIPGSDLAQYILPAMRNIYNALSSAGLQNQIKVSTAVDTGVLGTSYPPSAGAFSSAAQAYLSPIVQFLASNGAPLLVNVYPYFSYTGNPGQISLPYALFTASGVVVQDGRFSYQNLFDAIVDAVFAALERVGGANVAVVVSESGWPSAGGGAEASTSNAQTYNQNLIRHVGGGTPRRPGKEIEAYIFEMFNENCKAGGIEQNFGLFYPNKQPVYQISFKLTCKVDESSPTYLPTYENMKSTKIKGEFSWRKLNHDDIQTPLFTHYQYVTSFLKLERITIWTYKYFGSLFHTIYSKKDVFGVLNTVLQFARLPLQRQLQHAKKPSSAMGHVLHLDGATSSSFMFHFRSRALYIDHIKVWIASSFVTLYAALAYTLLPFRTLDCRK

SEQ ID NO: 22 - DNA, 7397 bp, Musa acuminata
  misc_feature 82, 601, 628, 640, 655, 692, 725, 774, 793, 806, 813,854, 867, 870, 876, 882, 890, 919, 946, 959, 965, 995, 999, 1002,1028, 1043, 1054, 1075, 1093, 1515, 2166, 2216, 2265, 2345,2533, 2870, 2917, 3077, 3337, 3356, 3618, 3627, 3754: n = A,T,C or G
  misc_feature 3810, 3819, 3884, 3893, 4494, 4503, 4524, 4533, 4568,4574, 4597, 4654, 4724, 4741, 4759, 4852, 5027, 5253, 5546, 5565,5567, 5575, 5578, 5618, 5619, 5650, 5669, 5672, 5677, 5683,5694, 5704, 5708, 5732, 5741, 5754, 5758, 5772, 5778: n = A,T,C or G
  misc_feature 5780, 5784, 5788, 5802, 5804, 5808, 5813, 5820, 5824,5832, 5834, 5836, 5854, 5858, 5863, 5872, 5875, 5889, 5915, 5922,5950, 5990, 6006, 6011, 6344, 6401, 6416, 6596, 6600, 6608,6612, 6712, 6748, 6753, 6756, 6762, 6830, 6844, 6847: n = A,T,C or G
  misc_feature 6863, 6910, 6965, 6968, 7070, 7116, 7179, 7291, 7322,7325, 7345, 7351, 7359, 7387, 7395: n = A,T,C or G
agcgaggtcgactaatgagctactaacattaatgtcacagatagtaatagatgagaagccgtatccaacacgcaatctgtanacttggtcacaggacttcttatccaaagactcgcctctgcgatttcccacattcacctcatttggtccataggaagcttcacagcgggcaggaatccatttctctatataagcaccacctcccacccacaccaccaccactaccactgctaaggaggatgaaggccttgttgttggtcatctttaccctggcctcgtcgctcggcgccttcgccgagcaatgcggaaggcaagccgggggggctctctgccccggcgggctgtgctgtagccagtacggctggtgcggtaacacggatccatactgcggccaaggatgccagagccaatgcggcggtagcggcggtagcggcggtggcagcgtggcctcgatcatcagctcctccctcttcgagcagatgctgaagcatcgcaacgacgcagcctgccccggcaagggtttctacacgtacaacgccttcatcgccgccgccaactccttcagcgggttcgggacgaccggcgacgacccaagaagaanaaggagatcgcggctttcttggcgcanacgtctcacganacgacaggtaattcncacatctcccgaagctcgtaaactgtttatgggatanaaaactgaatgtttggggtttggcaggtgggtnggcgacgcgcccgatggtccgtacgccttgggttactgcttcgtccaanaacaaaaccctcatcggantactgcgtcccanctcccantggccgtgcgctgcagcaaaaaatactacggccgaagcccntccaaatttcatngtnagccanattctnacagttcntcgccgcgatcgagttcacaacgatgccntttctaacgcaacaatccgatgtgttntgcgtgcagcaantacaantacgggccggccgggagagccatcggttcngacntgntcaacaacccagacctggtggccacngacgcgaccatctcnttcaagacggntctgtggttttggatgactcntcagtcgcccaagccgtngtgccacgacgtgataaccgggagctggacgccatccaacgccgaccaggcggccggaaggcttccgggctacggtgtcaccaccaacatcatcaatggagggttggagtgcgggaaagggtacgatgccagggtggcggataggatcggcttctacaagaggtactgcgacttgctgggggtgagctacggagacaacttggactgctacaaccagagaccctttgcttctacagcagctacagccacattctagcggtgagctatggagacaacttggagtgctacaaccagagaccctttacttagtccgatactactgtgacgaatccatgtaataacgcaataaacgctattactgagatagcgactccgtgagttgactgtagaagttgcggaggaagtcttcaataaaagcttanctacatacatggcccacaactatcgttgaccgtgatcatatgcatccatcaaatgtcctcaaatgtcttggagtaagtaaatgcgtattcgatcggtaaaatgaagatgttagaataaataaaattaattatttttttataattataaatattttaatatattttttaatcttaaagatcctaaaaatctaattataaggattttatatatggattgggatactaagaatatttaattataaaaattaatatactttttaatcttaaagatctaattataagtattttctatatggattgggatattaactcgatttacttataaaaattttaatataaaaattttaaatttaaaaattaaaatactaaaaatatctaaatataacggtaatcatgagatcgagaacgtgatgattgagatcatgagatcgaggttgagagtaaaaaggaaattacgttaatcatgggaaatttcgttttgtttgcacggtcgagatggtgaccgtggacacctaacatccacaaccggcatgcaataaccatgttgtcatatgttagcttgtctcatatcttatgaccatgaatcacatagtcttcacgaatattaattaagccagcttagcatcacagttttgcacctttgtaccatanctgaagtgttcgtatggcttgacccatcccgagtgtatggtctcccggancctggagcgtgttaacccgaggtctagttgaggggcatagaccttgttntcttaggcagaggttgaagatcactcctttagctatccgttgggtgcctatataaaggtcgaaatcatgagggggattcntaactcgacctattcaatatttgagctagcaagagttggagttacgtgtatgaggttcgacccccaatgctgttcctggggtcgcttttatacctattcctgcatgtgatcatacatagtagctttaatcatcttcagtcatcatcgtacgttgggtgcatgcattgtctaatttactcgattcaatntcgttcgacactgcttcctacctactatgtggcccaatacatagttgtattgtctcatacggcctcgagcaaagcgtgtgcagaggaactgtgtcaagtggttggctggcctcgggctcatggcattgagttggctcgatacaacacatcggcttagggataccatgccgagtctattgtggtagttgacatgtcatgtggggtggatgccaaaatatgctatatcattctctccctacaaaggagttgtgccataggagaatcgtggacacggcttgggttctgtggtcggtccttgttcgcctcagttgggtggattacttcatcaagttggccntctgttggctgggcaaagtacacttggtagggatggtcgagacaagnccaaggaaggttggctaagacttggttttcgacaatcaattgtttatgaggcgaatggtatccctccgttggggtgtctgctcgtttcgatttgttgcgatggattgtttgttgtaggaggcttggttcgattgctcttaagtcgggagaaggtatttgntaaggagttcaatttgaccatgttgaagtgaataaaaggacttgccaagaagtttggctcgaccgtgttaaagccagagaatgtgtatgtcgaggtctattcaaccatgtggaagctagagaatgcaccaattgtgaggtttggcttgctcacgtttaaagcagaaggatatacttgctacgaggtttgctcaaccatgtggaagcaatcaaatgcacttgctatgaggtttggcttgacttactcgacaatggacgctngtaagtgagaagggactanccaagacttagttggcaaggactagtcgatacttgctcgacaatagatgcctataggtaatggattgactgagacttagtcgacaaagactagctgagacttagtgggcaatggatgcctataagtaagaaaggatggctcgagattaataaagatcaaataattaatataaatttatcaaacacttaatggacgcatataagtgagaaaggacggatcgagattaataaagatcaaataattaatataagtttatcaaacncttattaanacattggacaaaagaggtactatgtaatattaaaattgggaggcacaaatattatttccaaatacttttctccttaagcccttcgccaccattgccattttaatctattttttctatataattatcncataacattcgtacatgagatatgacataaaccttcgacctgctttagtaaacatnttgattatngtgacaccagaagccataatattgcttaccttaacatgatggagatgaactttagttggtccaantatctaatnaatggaagtggacaagcacgatgactaggatggctacatgttcatgtgttgactttccaagtaatcaatcaagctggaatcgaataagacgattaaagtagggcgatgaccattaagttcaatgtcacgctcatcaacataattccaacaccgtgcagaaagatcttatcttacattgacttgcccatccggccgccggcatcgattggcggaaacgaagggtcagtctcccaattcacattcaaaggacgaattcattttcatcagatgagcacttcagtcctgcttgattatattttattattattattattattaattgaatggtaagtttacagaatatatagatattttagtttcaataaaatattttaaaaaatgataaagggagaaggtggatttgatcttaggatttttattgtgagcaataaaagtctttagttagaacttccaaaatgtgtcaaatgaaccctaataagtgggtttggtctatggttacgatgagatcagtatttgtatataaaaaaattatcaacttgatttttattttttaacccttaataagtggacatgatatatcataatcaaatcatgtgatgtntgatgagtnataacatattttttaataatnaaaattatnaatagagaaaaaataagattactatcccttctatngatgtnttataatattttaatccctttcnatatagattcacgtagaataagaaagattataatcgcatcaaatcaaatacagaatnaaatcatgcttttgacttaattcgaaaaataatcttcctctcttgataatatccttattgataagcattnttatatatatatatatntatatcaacttctaaaanatatttttaaattaattaaatttatcaaaataaaaagataaactaaattagttctgcatcataatgtagtaagtgtaagaacttgtgaaatanggatctagaacactgatagaaaattccaaaccattactagttctacttgatgaaaacaaaaccatataaaagaatcctcttatatatatatatatatatatactactttacttattctttggacgtacaacacaagtcaggaaaccgaaacaaaggtggcggaaagttggcagangctgaagagacttttcgtagaagtgaaggagacacacgtctataagaattgtcatgactatacgctgaagaaaaagaggggagagagagagaaggaagcgccactgttgaccggtcttgtccatgaggaattgtttgtcgactaatgagcagtacaaacatttgtgtcgacagatggcaacaaatgagaagcggtatcccaacacgcaatctgtagcctttggtcnccagacttatccaaagacttgcctctgcgatttcctcatgcgcctcatctgttccaaaggaagcttcacagcgggcaggaatccatttctctatataagcaccacctcccacccacaccaccaccaccaccaccactgctaaggaggatgaaggccttgttgctggtcatttttaccctggcctcgtcgctcggcgccttcgccgagcaatgcggaaggcaagccgggggggctctctgccccggcgggctgtgctgtagccagtacggctggtgcggtaacacggatccatnctgcggtcaaggatgccananccaatgcncangctccacgccctccccttccactccgagcggcggtggcanngttggctcgatcatcatctcctccctcttcnagcagatgctgaagcatcncancgacncagccngccccggcaanggcttctacncgtncaccgccttcatctccgccgccanctccttcancgggttcgggacnaccngcgaccactccacnaataanangganatcncggctttcttggtncngacntctcncgagacnacangtaatccntncntctcccgaggctcgtctncagnttatngatagacanctnaatgcattgggttnggcacgtgggtggtccaccgtgcccnatggccnttcgcgtggggttactgcttcgtccagnaacagaaccctcatcggactactgcgtcgccagctcgcantggccgtgcgctgcangcaanaaatactacggccgaagccccatccaaatctcattcaactacaactacgggccggccgggaaaaccatcggctccgacctgctcaacaacccagacctggtggccaccgacccgaccatctccttcaagacggctctgtggttctggatgactcctcagtcgcccaagccgtcgtgccacgacgtgataaccgggagctggacgccatccaacgccgaccgggcggccggaaggcttccgggctacggtgtcaccaccaacatcatcaatggagggttggagtgcgggaaagggtccgatgccagggtggcggataggatcggcttctacaanaggtactgcgacttgctgggggtgagctacggagacaacttggactgctacaaccanagtccctttacttantccgatactatgtgcgaatccatgtaataacgcaataaacgctactgctgaaatagcgactccgtgagttgattgtagaagttgcggaggaaatcttcaataaaagctaagctgaacaagttcatggccctcaatcatcgttgatcgtcgtcagatgcatccatcaaatgtcttggagtnagtnaatgcgtnttcnatcggtaaattgaagatgttagaataaataaaattatttattttttataattataaatattttaatatattttttaatcttaaagatcctaaaaaatctnattataaggattttatatatggattgggatactaanaaaanttnattatnaaaattaatatacttttaatcttaaggatcctaaaaaaacataattataaggattttctatatggatngggatactaacaanatntaattgtaaaaatttnaatataaaattgttaaatctaaaaattaaaatactaaaaatatatantaatcatgatatcgagaatgtggcgcttagatctcgagatcgaggttgagactanagnggaaattatgttaatcatgggaaattttcttttgtttccaagacgatgaccgtggaaacctaacatccgcaatcggtcatgcaataaccatgttatcatcantgaacttgtcgtcgtcatcttacggccacaaatcacagtcttctancaaggcacgaatattaatgagtccaacgtagtatctatattgttttacacttttataccgtantcgaggtgttcgcacgatttggcccatcccaagtgcataagatcattgatatgacctctacgttggagcgtgttaacccgagatctagttgagggggcataggtctcatttntctacgtggaggttaaagatcacctttattncancccttgtagattctaaactngaggtngatctctntaggagatcggtctcccttggaactctntaggggtncc

SEQ ID NO: 23 - DNA, 7397 bp, Musa acuminata
  misc_feature 82, 601, 628, 640, 655, 692, 725, 774, 793, 806, 813,854, 867, 870, 876, 882, 890, 919, 946, 959, 965, 995, 999, 1002,1028, 1043, 1054, 1075, 1093, 1515, 2166, 2216, 2265, 2345,2533, 2870, 2917, 3077, 3337, 3356, 3618, 3627, 3754: n = A,T,C or G
  misc_feature 3810, 3819, 3884, 3893, 4494, 4503, 4524, 4533, 4568,4574, 4597, 4654, 4724, 4741, 4759, 4852, 5027, 5253, 5546, 5565,5567, 5575, 5578, 5618, 5619, 5650, 5669, 5672, 5677, 5683,5694, 5704, 5708, 5732, 5741, 5754, 5758, 5772, 5778: n = A,T,C or G
  misc_feature 5780, 5784, 5788, 5802, 5804, 5808, 5813, 5820, 5824,5832, 5834, 5836, 5854, 5858, 5863, 5872, 5875, 5889, 5915, 5922,5950, 5990, 6006, 6011, 6344, 6401, 6416, 6596, 6600, 6608,6612, 6712, 6748, 6753, 6756, 6762, 6830, 6844, 6847: n = A,T,C or G
  misc_feature 6863, 6910, 6965, 6968, 7070, 7116, 7179, 7291, 7322,7325, 7345, 7351, 7359, 7387, 7395: n = A,T,C or G
tcgctccagctgattactcgatgattgtaattacagtgtctatcattatctactcttcggcataggtggtgcgttagacatntgaaccagtgtcctgaagaataggtttctgagcggagacgctaaagggtgtaagtggagtaaaccaggtatccttcgaagtgtcgcccgtccttaggtaaagagatatattcgtggtggagggtgggtgtggtggtggtgattggtgacgattcctcctacttccggaacaacaaccagtagaatgggaccggagcagcgagccgcggaagcggctcgttacgccttccgttcggcccccccgagagacggggccgcccgacacgacatcggtcatgccgaccacgccattgtgcctaggtatgacgccggttcctacggtctcggttacgccgccatcgccgccatcgccgccaccgtcgcaccggagctagtagtcgaggagggagaagctcgtctacgacttcgtagcgttgctgcgtcggacggggccgttcccaaagatgtgcatgttgcggaagtagcggcggcggttgaggaagtcgcccaagccctgctggccgctgctgggttcttcttnttcctctagcgccgaaagaaccgcctntgcagagtgctntgctgtccattaagngtgtagagggcttcgagcatttgacaaataccctatnttttgacttacaaaccccaaaccgtccacccanccgctgcgcgggctaccaggcatgcggaacccaatgacgaagcaggttnttgttttgggagtagcctnatgacgcagggtngagggtnaccggcacgcgacgtcgttttttatgatgccggcttcgggnaggtttaaagtancantcggtntaagantgtcaagnagcggcgctagctcaagtgttgctacggnaaagattgcgttgttaggctacacaanacgcacgtcgttnatgttnatgcccggccggccctctcggtagccaagnctgnacnagttgttgggtctggaccaccggtgnctgcgctggtagagnaagttctgccnagacaccaaaacctactgagnagtcagcgggttcggcancacggtgctgcactattggccctcgacctgcggtaggttgcggctggtccgccggccttccgaaggcccgatgccacagtggtggttgtattatttacctcccaacctcacgccctttcccatgctacggtcccaccgcctatcctagccgaagatgttctccatgacgctgaacgacccccactcgatgcctctgttgaacctgacgatgttggtctctgggaaacgaagatgtcgtcgatgtcggtgtaagatcgccactcgatacctctgttgaacctcacgatgttggtctctgggaaatgaatcaggctatgatgacactgcttaggtacattattgcgttatttgcgataatgactctatcgctgaggcactcaactgacatcttcaacgcctccttcagaagttattttcgaatngatgtatgtaccgggtgttgatagcaactggcactagtatacgtaggtagtttacaggagtttacagaacctcattcatttacgcataagctagccattttacttctacaatcttatttattttaattaataaaaaaatattaatatttataaaattatataaaaaattagaatttctaggatttttagattaatattcctaaaatatatacctaaccctatgattcttataaattaatatttttaattatatgaaaaattagaatttctagattaatattcataaaagatatacctaaccctataattgagctaaatgaatatttttaaaattatatttttaaaatttaaatttttaattttatgatttttatagatttatattgccattagtactctagctcttgcactactaactctagtactctagctccaactctcatttttcctttaatgcaattagtaccctttaaagcaaaacaaacgtgccagctctaccactggcacctgtggattgtaggtgttggccgtacgttattggtacaacagtatacaatcgaacagagtatagaatactggtacttagtgtatcagaagtgcttataattaattcggtcgaatcgtagtgtcaaaacgtggaaacatggtatngacttcacaagcataccgaactgggtagggctcacataccagagggcctnggacctcgcacaattgggctccagatcaactccccgtatctggaacaanagaatccgtctccaacttctattgaggaaatcgataggcaacccacggatatatttccagctttagtactccccctaagnattgagctggataagttataaactcgatcgttctcaacctcaatgcacatactccaagctgggggttacgacaaggaccccagcgaaaatatggataaggacgtacactagtatgtatcatcgaaattagtagaagtcagtagtagcatgcaacccacgtacgtaacagattaaatgagctaagttanagcaagctgtgacgaaggatggatgatacaccgggttatgtatcaacataacagagtatgccggagctcgtttcgcacacgtctccttgacacagttcaccaaccgaccggagcccgagtaccgtaactcaaccgagctatgttgtgtagccgaatccctatggtacggctcagataacaccatcaactgtacagtacaccccacctacggttttatacagatatagtaagagagggtgtttcctcaacacggtatcctcttagcacctgtgccgaacccaagacaccagccaggaacaagcggagtcaacccacctaatgaagtagttcaaccggnagacaaccgacccgtttcatgtgaaccatccctaccagctctgttcnggttccttccaaccgattctgaaccaaaagctgttagttaacaaatactccgcttaccatagggaggcaaccccacagacgagcaaagctaaacaacgctacctaacaaacaacatcctccgaaccaagctaacgagaattcagccctcttccataaacnattcctcaagttaaactggtacaacttcacttattttcctgaacggttcttcaaaccgagctggcacaatttcggtctcttacacatacagctccagataagttggtacaccttcgatctcttacgtggttaacactccaaaccgaacgagtgcaaatttcgtcttcctatatgaacgatgctccaaacgagttggtacaccttcgttagtttacgtgaacgatactccaaaccgaactgaatgagctgttacctgcgancattcactcttccctgatnggttctgaatcaaccgttcctgatcagctatgaacgagctgttatctacggatatccattacctaactgactctgaatcagctgtttctgatcgactctgaatcacccgttacctacggatattcattctttcctaccgagctctaattatttctagtttattaattatatttaaatagtttgtgaattacctgcgtatattcactctttcctgcctagctctaattatttctagtttattaattatattcaaatagtttgngaataattntgtaacctgttttctccatgatacattataattttaaccctccgtgtttataataaaggtttatgaaaagaggaattcgggaagcggtggtaacggtaaaattagataaaaaagatatattaatagngtattgtaagcatgtactctatactgtatttggaagctggacgaaatcatttgtanaactaatancactgtggtcttcggtattataacgaatggaattgtactacctctacttgaaatcaaccaggttnatagattanttaccttcacctgttcgtgctactgatcctaccgatgtacaagtacacaactgaaaggttcattagttagttcgaccttagcttattctgctaatttcatcccgctactggtaattcaagttacagtgcgagtagttgtattaaggttgtggcacgtctttctagaatagaatgtaactgaacgggtaggccggcggccgtagctaaccgcctttgcttcccagtcagagggttaagtgtaagtttcctgcttaagtaaaagtagtctactcgtgaagtcaggacgaactaatataaaataataataataataataattaacttaccattcaaatgtcttatatatctataaaatcaaagttattttataaaattttttactatttccctcttccacctaaactagaatcctaaaaataacactcgttattttcagaaatcaatcttgaaggttttacacagtttacttgggattattcacccaaaccagataccaatgctactctagtcataaacatatatttttttaatagttgaactaaaaataaaaaattgggaattattcacctgtactatatagtattagtttagtacactacanactactcantattgtataaaaaattattanttttaatanttatctcttttttattctaatgatagggaagatanctacanaatattataaaattagggaaagntatatctaagtgcatcttattctttctaatattagcgtagtttagtttatgtcttantttagtacgaaaactgaattaagctttttattagaaggagagaactattataggaataactattcgtaanaatatatatatatatanatatagttgaagattttntataaaaatttaattaatttaaatagttttatttttctatttgatttaatcaagacgtagtattacatcattcacattcttgaacactttatncctagatcttgtgactatcttttaaggtttggtaatgatcaagatgaactacttttgttttggtatattttcttaggagaatatatatatatatatatatatgatgaaatgaataagaaacctgcatgttgtgttcagtcctttggctttgtttccaccgcctttcaaccgtctncgacttctctgaaaagcatcttcacttcctctgtgtgcagatattcttaacagtactgatatgcgacttctttttctcccctctctctctcttccttcgcggtgacaactggccagaacaggtactccttaacaaacagctgattactcgtcatgtttgtaaacacagctgtctaccgttgtttactcttcgccatagggttgtgcgttagacatcggaaaccagnggtctgaataggtttctgaacggagacgctaaaggagtacgcggagtagacaaggtttccttcgaagtgtcgcccgtccttaggtaaagagatatattcgtggtggagggtgggtgtggtggtggtggtggtggtgacgattcctcctacttccggaacaacgaccagtaaaaatgggaccggagcagcgagccgcggaagcggctcgttacgccttccgttcggcccccccgagagacggggccgcccgacacgacatcggtcatgccgaccacgccattgtgcctaggtangacgccagttcctacggtntnggttacgngtncgaggtgcgggaggggaaggtgaggctcgccgccaccgtnncaaccgagctagtagtagaggagggagaagntcgtctacgacttcgtagngtngctgngtcggncggggccgttnccgaagatgngcangtggcggaagtagaggcggcggtngaggaagtngcccaagccctgntggncgctggtgaggtgnttattntncctntagngccgaaagaaccangnctgnagagngctctgntgtncattaggnangnagagggctccgagcagangtcnaatanctatctgtnganttacgtaacccaanccgtgcacccaccaggtggcacgggntaccggnaagcgcaccccaatgacgaagcaggtcnttgtcttgggagtagcctgatgacgcagcggtcgagcgtnaccggcacgcgacgtncgttntttatgatgccggcttcggggtaggtttagagtaagttgatgttgatgcccggccggcccttttggtagccgaggctggacgagttgttgggtctggaccaccggtggctgggctggtagaggaagttctgccgagacaccaagacctactgaggagtcagcgggttcggcagcacggtgctgcactattggccctcgacctgcggtaggttgcggctggcccgccggccttccgaaggcccgatgccacagtggtggttgtagtagttacctcccaacctcacgccctttcccaggctacggtcccaccgcctatcctagccgaagatgttntccatgacgctgaacgacccccactcgatgcctctgttgaacctgacgatgttggtntcagggaaatgaatnaggctatgatacacgcttaggtacattattgcgttatttgcgatgacgactttatcgctgaggcactcaactaacatcttcaacgcctcctttagaagttattttcgattcgacttgttcaagtaccgggagttagtagcaactagcagcagtctacgtaggtagtttacagaacctcantcanttacgcanaagntagccatttaacttctacaatcttatttattttaataaataaaaaatattaatatttataaaattatataaaaaattagaatttctaggattttttagantaatattcctaaaatatatacctaaccctatgattnttttnaantaatanttttaattatatgaaaattagaattcctaggatttttttgtattaatattcctaaaagatatacctanccctatgattgttntanattaacatttttaaanttatattttaacaatttagatttttaattttatgatttttatatatnattagtactatagctcttacaccgcgaatctagagctctagctccaactctgatntcncctttaatacaattagtaccctttaaaagaaaacaaaggttctgctactggcacctttggattgtaggcgttagccagtacgttattggtacaatagtagtnacttgaacagcagcagtagaatgccggtgtttagtgtcagaagatngttccgtgcttataattactcaggttgcatcatagatataacaaaatgtgaaaatatggcatnagctccacaagcgtgctaaaccgggtagggttcacgtattctagtaactatactggagatgcaacctcgcacaattgggctctagatcaactcccccgtatccagagtaaanagatgcacctccaatttctagtggaaataangtngggaacatctaagatttganctccanctagaganatcctctagccagagggaaccttgaganatccccangg

SEQ ID NO: 24 - protein, 2326 aa, Musa acuminata
  VARIANT 25, 164, 173, 177, 181, 193, 204, 220, 227, 231, 233,247, 251, 254, 259, 269, 278, 282, 284, 294, 296, 305, 310, 314,320, 326, 458, 656, 673, 687, 713, 774, 883, 899, 952, 1038,1043, 1163, 1180, 1183, 1202, 1204, 1397, 1400, 1412: Xaa = Any Amino Acid
  VARIANT 1414, 1422, 1441, 1462, 1468, 1474, 1504, 1559, 1729,1735, 1736, 1739, 1740, 1753, 1764, 1770, 1771, 1773, 1775, 1778,1782, 1783, 1791, 1794, 1800, 1806, 1807, 1808, 1810, 1815,1818, 1822, 1825, 1826, 1832, 1833, 1835, 1837, 1838: Xaa = Any Amino Acid
  VARIANT 1842, 1851, 1863, 1876, 1881, 1883, 1991, 2009, 2014,2071, 2075, 2109, 2120, 2121, 2122, 2124, 2147, 2151, 2152, 2157,2169, 2187, 2188, 2221, 2236, 2257, 2293, 2303, 2304, 2310,2312, 2323: Xaa = Any Amino Acid
SEVDATNINVTDSNREAVSNTQSVXLVTGLLIQRLASAISHIHLIWSIGSFTAGRNPFLYISTTNAEGKPGGLSAPAGCAVASTAGAVTRIHTAAKDARANAAVAAVAAVAAWPRSSAPPSSSRCSIATTQPAPARVSTRTTPSSPPPTPSAGSGRPATTQEEXGDRGFLGAXVSRXDRFXHLPKLVNCLWDXKLNVWGLAGGXATRPMVRTPWVTASSXNKTLIGXLRPXSXWPCAAAKNTTAEAXPNFXVSXILTVXRRDRVHNDAXSNATIRCVXRAAXTXTGRPGEPSVXTXSTTQTWWPXTRPSXSRRXCGFGLXSRPSRXATTPGAGRHPTPTRRPEGFRATVSPPTSSMEGWSAGKGTMPGWRIGSASTRGTATCWGATETTWTATTRDPLLLQQLQPHSSGELWRQLGVLQPETLYLVRYYCDESMRNKRYYDSDSVSLKLRRKSSIKAXLHTWPTTIVDRDHMHPSNVLKCLGVSKCVFDRNEDVRINKINYFFIIINILIYFLILKILKILGFYIWIGILRIFNYKNYTFSRSNYKYFLYGLGYLDLLIKILIKFIKLKYKYLNITVIMRSRTLRSDRGEKGNYVNHGKFRFVCTVEMVTVDTHPQPACNNHVVICLVSYLMTMNHIVFTNINASLASQFCTFVPXLKCSYGLTHPECMVSRXLERVNPRSSGATLXSAEVEDHSFSYPLGAYIKVEIMRGIXNSTYSIFELARVGVTCMRFDPQCSSWGRFYTYSCMSYIVALIIFSHHRTLGACIVFTRFNXVRHCFLPTMWPNTLYCLIRPRAKRVQRNCVKWLAGLGLMALSWLDTTHRLRDTMPSLLWLTCHVGWMPKYAISFSPYKGVVPENRGHGLGSVVGPCSPQLGGLLHQVGXLLAGQSTLGRDGRDKXKEGWLRLGFRQSIVYEANGIPPLGCLLVSICCDGLFVVGGLVRLLLSREKVFXKEFNLTMLKIKGLAKKFGSTVLKPENVYVEVYSTMWKLENAPIVRFGLLTFKAEGYTCYEVCSTMWKQSNALAMRFGLTYSTMDAXKEGTXQDLVGKDSILARQMPIGNGLTETSTKTSDLVGNGCLVRKDGSRLIKIKLIIYQTLNGRIVRKDGSRLIKIKLIVYQTLITLDKRGTMYNWEAQILFPNTFLLKPFATIAILIYFFYIIIXHSYMRYDINLRPALVNXLIXVTPEAIILTLTWRTLVGPXIXMEVDKHDDDGYMFMCLSKSIKLESNKTIKVGRPLSSMSRSSTFQHRAERSYLTLTCPSGRRHRLAETKGQSPNSHSKDEFIFIRALQSCLIIFYYYYYYLNGKFTEYIDILVSIKYFKKREKVDLILGFLLAIKVFSNFQNVSNEPVGLVYGYDEISICIKNYQLDFYFLTLNKWTYIIIKSCDVVITYFLIXKIXNREKIRLLSLLXMXYNILIPFXIDSRRIRKIIIASNQIQNXIMLLTFEKSSSLDNILIDKHXYIYIYXYQLLKXIFKLIKFIKIKRTKLVLHHNVVSVRTCEIXINTDRKFQTITSSTKQNHIKESSYIYIYIYTTLLILWTYNTSQETETKVAESWQXLKRLFVEVKETHVYKNCHDYTLKKKRGEREKEAPLLTGLVHEELFVDAVQTFVSTDGNKEAVSQHAICSLWSPDLSKDLPLRFPHAPHLFQRKLHSGQESISLYKHHLPPTPPPPPPLLRRMKALLLVIFTLASSLGAFAEQCGRQAGGALCPGGLCCSQYGWCGNTDPXCGQGCXXQCXXSTPSPSTPSGGGXVGSIIISSLFXQMLKHXXDXAXPGXGFYXXTAFISAAXSFXGFGTTXDHSTNXXXIXAFLVXTSXETTXNPXXSRGSSXXYXTXXCIGXGTWVVHRAXWPFAWGYCFVQXQNPHRTTASPARXGRALXAXNTTAEAPSKSHSTTTTGRPGKPSAPTCSTTQTWWPPTRPSPSRRLCGSGLLSRPSRRATTPGAGRHPTPTGRPEGFRATVSPPTSSMEGWSAGKGPMPGWRIGSASTXGTATCWGATETTWTATTXVPLLXPILCANPCNNAINATAEIATPVDCRSCGGNLQKLSTSSWPSIIVDRRQMHPSNVLEXVNAXSIGKLKMLEIKLFIFYNYKYFNIFFNLKDPKKSXYKDFIYGLGYXXXIXKINILLILRILKKHNYKDFLYGXGYQXXIVKIXINCIKLKYKYIXIMISRMWRLDLEIEVETXXEIMLIMGNFLLFPRRPWKPNIRNRSCNNHVIIXELVVVILRPQITVFXQGTNINESNVVSILFYTFIPXSRCSHDLAHPKCIRSLIPLRWSVLTRDLVEGAVSFXYVEVKDHLYXXPCRFTXGXSLEIGLPWNSXGVP

SEQ ID NO: 25 - protein, 2258 aa, Musa acuminata
  VARIANT 27, 160, 169, 173, 190, 200, 216, 222, 227, 229, 243,247, 248, 250, 252, 255, 273, 278, 280, 290, 291, 292, 301, 306,459, 654, 669, 685, 710, 767, 878, 893, 944, 1029, 1104,1107, 1162, 1164, 1182, 1185, 1372, 1375, 1379, 1382, 1393: Xaa = Any Amino Acid
  VARIANT 1395, 1417, 1440, 1445, 1450, 1475, 1533, 1666, 1672,1673, 1676, 1690, 1706, 1707, 1714, 1719, 1727, 1730, 1734, 1740,1742, 1743, 1744, 1750, 1751, 1752, 1754, 1756, 1757, 1760,1761, 1769, 1770, 1773, 1779, 1788, 1790, 1799, 1813: Xaa = Any Amino Acid
  VARIANT 1818, 1820, 1930, 1949, 1954, 2011, 2012, 2015, 2016,2058, 2060, 2061, 2063, 2083, 2087, 2088, 2092, 2106, 2122, 2123,2156, 2171, 2191, 2224, 2235, 2236, 2247, 2258: Xaa = Any Amino Acid
ARSTNELLTLMSQIVIDEKPYPTRNLXTWSQDFLSKDSPLRFPTFTSFGPEASQRAGIHFSIAPMRKASRGGSLPRRAVLPVRLVRHGSILRPRMPEPMRRRRRRWQRGLDHQLLPLRADAEASQRRSLPRQGFLHVQRLHRRRQLLQRVRDDRRRPKKXKEIAAFLAXTSHXTTGNSHISRSSTVYGIXNMFGVWQVGXRRARWSVRLGLLLRPXTKPSSXYCVPXPXGRALQQKILRPKPXQISXXAXFXQFXAAIEFTTMPFLTQQSDVXCVQQXQXRAGRESHRFXXXQQPRPGGHXRDHLXQDGSVVLDDSSVAQAVVPRRDNRELDAIQRRPGGRKASGLRCHHQHHQWRVGVRERVRCQGGGDRLLQEVLRLAGGELRRQLGLLQPETLCFYSSYSHILAVSYGDNLECYNQRPFTSDTTVTNPCNNAINAITEIATPVDCRSCGGSLQKLXYIHGPQLSLTVIICIHQMSSNVLEVNAYSIGKMKMLEIKLIIFLLIFYIFSRSKSNYKDFIYGLGYEYLIIKINILFNLKDLIISIFYMDWDINSIYLKFYKNFKFKNNTKNIIRSDRERDDDHEIEVESKKEITLIMGNFVLFARSRWPWTPNIHNRHAITMLSYVSLSHILPITSSRILIKPAHHSFAPLYHXSVRMAPIPSVWSPGXWSVLTRGLVEGHRPCXLRQRLKITPLAIRWVPIRSKSGGFXTRPIQYLSQELELRVGSTPNAVPGVAFIPIPACDHTLSSSVIIVRWVHALSNLLDSXSFDTASYLLCGPIHSCIVSYGLEQSVCRGTVSSGWLASGSWHVGSIQHIGLGIPCRVYCGSHVMWGGCQNMLYHSLPTKELCHRRIVDTAWVLWSVLVRLSWVDYFIKLAXCWLGKVHLVGMVETXPRKVGDLVFDNQLFMRRMVSLRWGVCSFRFVAMDCLLEAWFDCSVGRRYLXRSSIPCSEKDLPRSLARPCSQRMCMSRSIQPCGSRMHQLGLACSRLKQKDILATRFAQPCGSNQMHLLGLALTRQWTLVSEKGLXKTLARTSRYLLDNRCLVMDLRLSRQRLAETWAMDAYKERMARDRSNNYKFIKHLMDAYKERTDRDRSNNYKFIKXLLXHWTKEVLCNIKIGRHKYYFQILFSLSPSPPLPFSIFSILSHNIRTDMTTFDLLTXLXHQKPYCLPHDGDELLVQXSNXWKWTSTMTRMATCSCVDFPSNQSSWNRIRRLKGDDHVQCHAHQHNSNTVQKDLILHLAHPAAGIDWRKRRVSLPIHIQRTNSFSSDEHFSPALYFIIIIIINMVSLQNIIFFQNILKNDKGRRWISDFYCEQKSLVRTSKMCQMNPNKWVWSMVTMRSVFVYKKIINLIFIFPLISGHDISSNHVMXDEXHIFXKLXIEKKDYYPFYXCXIIFSLSIIHVEERLSHQIKYRXKSCFLNSKNNLPLLIISLLISIXIYIYXYINFXIFLNLNLSKKDKLNFCIIMVELVKXGSRTLIENSKPLLVLLDENKTIKNPLIYIYIYILLYLFFGRTTQVRKPKQRWRKVGRXRDFSKRRHTSIRIVMTIRRKRGERERRKRHCPVLSMRNCLSTNEQYKHLCRQICSKGSFTAGRNPFLYISTTSHPHHHHHHHCGGRPCCWSFLPWPRRSAPSPSNAEGKPGGLSAPAGCAVASTAGAVTRIHXAVKDAXXNAXAPRPPLPLRAAVAXLARSSSPPSSSRCSIXXTQPAPAXASTRXPPSSPPPXPSXGSGXPATTPXIXXXSRLSWXXXLXRXXVIXXSPEARLQXXDRXLNALGXARGWSTVPXGXSRGVTASSXNRTLIGLLRRQLAXAVRCXQXILRPKPHPNLIQLQLRAGRENHRLRPAQQPRPGGHRPDHLLQDGSVVLDDSSVAQAVVPRRDNRELDAIQRRPGGRKASGLRCHHQHHQWRVGVRERVRCQGGGDRLLQXVLRLAGGELRRQLGLLQPXSLYLXRYYVRIHVITQTLLLKRLRELIVEVAEEIFNKSAEQVHGPQSSLIVVRCIHQMSWSXXMRXXSVNRCNKNYLFFIIINILIYFLILKILKNLIIRILYMDWDTXKXXLXKLIYFSGSKNIIIRIFYMDXDTNXXLKFXYKIVKSKNNTKNIXSYRECGAISRSRLRLXXKLCSWEIFFCFQDDDRGNLTSAIGHAITMLSSXNLSSSSYGHKSQSSXKARILMSPTYLYCFTLLYRXRGVRTIWPIPSADHYDLYVGACPEILRGHRSHXSTWRLKITFIXXLVDSKLEVDLXRRSVSLGTLGX

SEQ ID NO: 26 - protein, 2359 aa, Musa acuminata
  VARIANT 23, 196, 205, 209, 214, 226, 254, 260, 264, 267, 284,285, 287, 289, 301, 309, 313, 315, 327, 328, 345, 352, 358, 494,697, 713, 728, 813, 939, 991, 1076, 1081, 1165, 1167, 1208,1226, 1229, 1250, 1253, 1442, 1445, 1452, 1455, 1465: Xaa = Any Amino Acid
  VARIANT 1474, 1492, 1511, 1517, 1523, 1549, 1604, 1676, 1770,1777, 1780, 1781, 1794, 1795, 1805, 1812, 1814, 1816, 1820, 1823,1832, 1835, 1840, 1841, 1846, 1847, 1849, 1850, 1855, 1857,1861, 1862, 1864, 1865, 1871, 1872, 1874, 1877, 1878: Xaa = Any Amino Acid
  VARIANT 1883, 1894, 1903, 1916, 1922, 1923, 2034, 2053, 2058,2114, 2118, 2144, 2155, 2157, 2158, 2160, 2179, 2184, 2185, 2190,2206, 2223, 2224, 2256, 2271, 2290, 2326, 2335, 2336, 2342,2344, 2347, 2356, 2359: Xaa = Any Amino Acid
QRGRLMSYHCHRMRSRIQHAICXLGHRTSYPKTRLCDFPHSPHLVHRKLHSGQESISLYKHHLPPTPPPLPLLRRMKALLLVIFTLASSLGAFAEQCGRQAGGALCPGGLCCSQYGWCGNTDPYCGQGCQSQCGGSGGSGGGSVASIISSSLFEQMLKHRNDAACPGKGFYTYNAFIAAANSFSGFGTTGDDPRRXRRSRLSWRXRLTXRQVIXTSPEARKLFMGXKTECLGFGRWVGDAPDGPYALGYCFVQXQNPHRXTASXLPXAVRCSKKYGRSPSKFHXXPXSXSSSPRSSSQRCXFRNNPMCXACSXYXYGPAGRAIGSDXXNNPDLVATDATISFKTXLWFWMTXQSPKPXCHDVITGSWTPSNADQAAGRLPGYGVTTNIINGGLECGKGYDARVADRIGFYKRYCDLLGVSYGDNLDCYNQRPFASTAATATFRAMETTWSATTRDPLLSPILLRIHVITQTLLLRRLRELTVEVAEEVFNKSLXTYMAHNYRPSYASIKCPQMSWSKMRIRSVKRCNKNLFFYNYKYFNIFFNLKDPKNLIIRILYMDWDTKNILKLIYFLILKILVFSIWIGILTRFTYKNFNIKILNLKIKILKISKYNGNHEIENVMIEIMRSRLRVKRKLRSWEISFCLHGRDGDRGHLTSTTGMQPCCHMLACLISYDHESHSLHEYLSQLSITVLHLCTIXEVFVWLDPSRVYGLPXPGACPEVLRGIDLVXLGRGRSLLLSVGCLYKGRNHEGDSLDLFNIASKSWSYVYEVRPPMLFLGSLLYLFLHVIIHSSFNHLQSSSYVGCMHCLIYSIQXRSTLLPTYYVAQYIVVLSHTASSKACAEELCQVVGWPRAHGIELARYNTSAGYHAESIVVVDMSCGVDAKICYIILSLQRSCAIGESWTRLGFCGRSLFASVGWITSSSWPSVGWAKYTWGWSRQXQGRLAKTWFSTINCLGEWYPSVGVSARFGLLRWIVCCRRLGSIALKSGEGIXGVQFDHVEVNKRTCQEVWLDRVKARECVCRGLFNHVEARECTNCEVWLAHVSRRIYLLRGLLNHVEAIKCTCYEVWLDLLDNGRXVRRDXPRLSWQGLVDTCSTIDAYRWIDDLVDKDLRLSGQWMPISKKGWLEINKDQIININLSNTWTHISEKGRIEINKDQIINISLNSXYXIGQKRYYVILKLGGTNIISKYFSPALRHHCHFNLFFLYNYXITFVHEIHKPSTCFSKHXDYXDTRSHNIAYLNMMEMNFSWSXYLXNGSGQARLGWLHVHVLTFQVINQAGIEDDSRAMTIKFNVTLINIIPTPCRKILSYIDLPIRPPASIGGNEGSVSQFTFKGRIHFHQMSTSVLLDYILLLLLLLIEWVYRIYRYFSFNKIFKMIKGEGGFDLRIFIVSNKSLLELPKCVKTLISGFGLWLRDQYLYIKKLSTFLFFNPVDMIYHNQIMCXMSXNIFFNNXNYXRKNKITIPSXDVLYFNPFXYRFTNKKDYNRIKSNTEXNHAFDLIRKIIFLSYPYAXLYIYIXISTSKXYFINIYQNKKINISSASCSKCKNLNXDLEHKIPNHYFYLMKTKPYKRILLYIYIYIYYFTYSLDVQHKSGNRNKGGGKLAXAEETFRRSEGDTRLELSLYAEEKEGREREGSATVDRSCPGIVCRLMSSTNICVDRWQQMRSGIPTRNLPLVXRLIQRLASAISSCASSVPKEASQRAGIHFSIAPPPTHTTTTTTTAKEDEGLVAGHFYPGLVARRLRRAMRKASRGGSLPRRAVLPVRLVRHGSXLRSRMPXPMXXLHALPFHSERRWXXWLDHHLLPLXADAEASXRXSXPRQXLLXVHRLHLRRXLLXRVRDXXRPLHXXGXXGFLGXDXSRDXXSXXLPRLVXXLXIDXXMHWVXHVGGPPCPMAXRVGLLLRPXTEPSSDYCVASSXWPCAAXXKYYGRSPIQISFNYNYGPAGKTIGSDLLNNPDLVATDPTISFKTALWFWMTPQSPKPSCHDVITGSWTPSNADRAAGRLPGYGVTTNIINGGLECGKGSDARVADRIGFYXRYCDLLGVSYGDNLDCYNXSPFTXSDTMCESMRNKRYCNSDSVSLKLRRKSSIKAKLNKFMALNHRSSSDASIKCLGVSXCVFXRIEDVRINKIIYFLLIFYIFSRSKIXLGFYIWIGILXKXXYXNYTFNLKDPKKTLGFSIWXGILTXXNCKNXNIKLLNLKIKILKIYXNHDIENVALRSRDRGDXXGNYVNHGKFSFVSKTMTVETHPQSVMQPCYHXTCRRHLTATNHSLLXRHEYVQRSIYIVLHFYTVXEVFARFGPSQVHKIIDMTSTLERVNPRSSGGIGLIXLRGGRSPLXXPLILNXRXISXGDRSPLELXRGX

SEQ ID NO: 27 - DNA, 4924 bp, Musa acuminata
  misc_feature 879, 3691, 4119: n = A,T,C or G
ggatcccaacttttaggaatggatcttaaaattttagttataagttcaaagttagaaaaatctttaccaagagctttgagtccattgatgacatccgtgaaacggtgtacatgtctccgatggactcacttggtttcattcggaaaagttcgaaagagtgcataagaatattgattttggattctttcactcggttggtgccttcatgagtgacctcaagagtcctccaaatatcaaaagccgaatcacaaattgaaatgtgattgaattcatttttgtctaatgcacaaaacagggcattcatagcctttgtgtttaaagcaaaaacattcttctccgattcatcccattcgctcatcggaagagaaaatttttgaaatccattttcgacaatagaccaaagctcgaaatccatggaaatgaggaagatcctcatatgagttttccaatacatgtaattcgactcattaaacataggtggatgtgtaatgaaatgaccctcatgcsctatctctcttgggtattaaaccaaatatgagagtgagccttgctctgataccaattgttaggatcagagtggcactaagagagggggggagtgaattagtgcagtggattaaaacttataagtttaaaaatgaattcgtaaatacgagaagatttcgttttaatagtaacttgagtagatgaaaaccaaaagttaacagtagtgtaaataacaatttcgggaaagtaagaactcacacattcaaggaacataccaatttaaagtggttcggtcaaaatgacctacatccacttgtgaagccttcttcgaagaggctcccaacttccactagcaaatcactttgaaggggaaggacaaatacctctcttacnaccttttacaatggttcatactcttacaaattttcaacgagaaagaaggaggtgaacatgcaagcaattgaaaacaagacttgctaaagactttgctaaggctttttttctcaatctattgcttctcaaaagttgtattctctgctgagaattgaggggtatttatagaccccaagaggatttaaatttgggctccaaatttcgaatgctcttgggttcccgaggttgccggtgccaccgcctgtcagtgtttgacactggacagtgtactagcggtgccaccgccggacctctcgggtgttgggcggtgccaccgcctagactttttcagctcactggttggattccaaacttgacccaaaccagtccgaactcgggtccaattgacccgtaaccggattataggattaacccttaatcctaaccctaattatatgcaaactacgcaactgaaaatatagtcctaagcaagtttttaaccggcaaacgtcgagtcttcttccggcgatctttcggcagacttctgatatacctttggatttcttctagcggactcctagtagggtcccgatcttgtggcgagtttagcgagtagccgaaccttctcggtgatctccgcaaaccgccgatgatctcttcggcagactttcgaaaacttcgacaagtccccgatttcttctcggttggttccgacagcatctctaacgaaacttcggactccttgaatgtccatcgaacttgactccggtaggcttgctttatattttcaggctatcatagttaatcctacatacttaactcaataatatggattagattaattaacccatcaattgatttcatcatcaaaattcgacattcaacaaacatccgtactcaataacccatcaggctatagttacgtgactatctactgtgatccgtacgtgaagttagcgagtcatgatccaggtcgtgtcacttattggccgaacacgtatcccttatccaaatccagtcttctcaactcttctagcctacccgtctctttttttattacttttgaaagaattcaaatcaaaacagatacaaaataacacggtgagacactgtgacatgctagtctctggaaagcattaattcgcgcatccacagacgtcgtcagcttcatcacccactttttcctacataaccatgtcgcatggctttgttgatgacagaccaccacaagcttgcctttggttgtgcctaacagagagagagagacagaccgatagcctcctcattcactatggcgatccgatcgccagcttcgctgctgttatttgcgttcctgatgcttgcgctcacgggaagactgcaggcccggcgcagctcatgcattggcgtctactggggacaaaacaccgacgagggaagcttagcagatgcttgtgccacaggcaactacgaatacgtgaacatcgccacccttttcaagtttggcatgggccaaactccagagatcaacctcgccggccactgtgaccctcggaacaacggctgcgcgcgcttgagcagcgaaatccagtcctgccaggagcgtggcgtcaaggtgatgctctccatcggaggtggcgggtcttatggcctgagttccaccgaagacgccaaggacgtagcgtcatacctctggcacagtttcttgggtggttctgctgctcgctactcgagacccctcggggatgcggttctggatggcatagacttcaacatcgccggagggagcacagaacactatgatgaacttgccgctttcctcaaggcctacaacgagcaggaggccggaacgaagaaagttcacttgagtgctcgtccgcagtgtcctttcccggattactggcttggcaacgcactcagaacagatctcttcgacttcgtgtgggtgcagttcttcaacaacccttcgtgccatttctcccagaacgctatcaatcttgcaaatgcgttcaacaattgggtcatgtccatccctgcgcaaaagctgttccttgggcttcctgctgctcctgaggctgctccaactggtggctacattccaccccatgatctcatatctaaagttcttccgatcctaaaggattccgacaagtacgcaggaatcatgctgtggactagataccacgacagaaactccggctacagttctcaagtcaagtcccacgtgtgtccagcgcgtcggttctccaacatcttatctatgccggtgaagtcttccaagtaaacctgaacggcgtagatgatcggtggtcgaaaactccgatcatcatgggtccccatccgtatccgtgcgttgctacgttatggtgtttcccttgtatgttggtcttttcaataatataataaggggttagttttacgtttccatattttccatgttcgaaaacagtatatttgctgccccttccaaatttgaaaaagataaaataaatatataactaaaaatatcctcttttttttttctttcgacaaatatataactcttaactttcccaattgtttaagcaaaagatataaatcctcttccacacaaaagacgaatccatgattgctggattgctgtctactggtgccgaaatggcgacgagagaagcttgtgctacctgcaattacaagttcgtcaacattgtcttccttgccatgtttggtgacgccatactcccgtgatcaggacacacctctggaacagtttcttgggaagttaatcttcttctcggctcctcggcgaccaatcttgtgaggttcttctcctgaatggtgtccacttcgacatcgaaggtctacctgagcgcanatccacagttccgactacgtgtgggtgcagttctactacacaggcaactcgcagatgcccggtaacaatgggttctccatcctgcatggaaggtgttccctggacttcctgctgctcctcaggctgctggaaggagctccattccactagtgatcttacacgtgtcttatcatcaagaattatagcaagtaccgagggattattaaaataaaaaaaaagggaagaatgggaattagaattaaaactgaaaccggccatgaagaacgtttcgagtgaagacaaacgacagtatgagacggtagtttgctatggacatggatcgttcccaaagcagtccaagtctttatgaaccggtctatcggttcagccttcaagaaccgcgaggataaccggcccaagagaaacaacaaattgtggtgagcttttantataaaccgaacggtgccgtccgtcagatgttaaatggacggcggatagatctccagagtaaatctgaggaaaatcgttccggcccccctaccacgacccacgcgatccgtcctctcccccaccccctacacctttttcttcttccgctcctgcgatcggttatttgattttgtgtatgatatccaatttcttttctggagtggtatcctattctaatttcttagattgttgtattgaaccatcagttttggtttaagcgcatgatggcggagagtttcgggagatgggagtcagatcccttgttttctgctgccgaagtggtgcaagattcggccgataggttttttctctcattttaagctcaattatgcggtcattcttgttaggctttggagaatttgctctatttcgaaagaaattgctgctttctagttttgattagtccctataaaatttgctttcggttctgaatatccgagaatgtcgtatcgtcaatgacgattcttttttagaattctaatactttgtcctgttttctgtgatttaatggagaaaatattgttccttttagtgatctatgctctcccgaccattaggatgagggttgaaggtgaaaatactttctggtaattttcctctctaaattcttccaaacacgacacaagtataattatagaccaagattgattcttcttatgcaccgattctcacttcccttccctctgtgttatggttatcgttgttactgatggttgcttaactcatggggtagcgcctgggtgatccgttgacctgcaggtcgac

SEQ ID NO: 28 - DNA, 4924 bp, Musa acuminata
  misc_feature 879, 3691, 4119: n = A,T,C or G
cctagggttgaaaatccttacctagaattttaaaatcaatattcaagtttcaatctttttagaaatggttctcgaaactcaggtaactactgtaggcactttgccacatgtacagaggctacctgagtgaaccaaagtaagccttttcaagctttctcacgtattcttataactaaaacctaagaaagtgagccaaccacggaagtactcactggagttctcaggaggtttatagttttcggcttagtgtttaactttacactaacttaagtaaaaacagattacgtgttttgtcccgtaagtatcggaaacacaaatttcgtttttgtaagaagaggctaagtagggtaagcgagtagccttctcttttaaaaactttaggtaaaagctgttatctggtttcgagctttaggtacctttactccttctaggagtatactcaaaaggttatgtacattaagctgagtaatttgtatccacctacacattactttactgggagtacgsgatagagagaacccataatttggtttatactctcactcggaacgagactatggttaacaatcctagtctcaccgtgattctctccccccctcacttaatcacgtcacctaattttgaatattcaaatttttacttaagcatttatgctcttctaaagcaaaattatcattgaactcatctacttttggttttcaattgtcatcacatttattgttaaagccctttcattcttgagtgtgtaagttccttgtatggttaaatttcaccaagccagttttactggatgtaggtgaacacttcggaagaagcttctccgagggttgaaggtgatcgtttagtgaaacttccccttcctgtttatggagagaatgntggaaaatgttaccaagtatgagaatgtttaaaagttgctctttcttcctccacttgtacgttcgttaacttttgttctgaacgatttctgaaacgattccgaaaaaaagagttagataacgaagagttttcaacataagagacgactcttaactccccataaatatctggggttctcctaaatttaaacccgaggtttaaagcttacgagaacccaagggctccaacggccacggtggcggacagtcacaaactgtgacctgtcacatgatcgccacggtggcggcctggagagcccacaacccgccacggtggcggatctgaaaaagtcgagtgaccaacctaaggtttgaactgggtttggtcaggcttgagcccaggttaactgggcattggcctaatatcctaattgggaattaggattgggattaatatacgtttgatgcgttgacttttatatcaggattcgttcaaaaattggccgtttgcagctcagaagaaggccgctagaaagccgtctgaagactatatggaaacctaaagaagatcgcctgaggatcatcccagggctagaacaccgctcaaatcgctcatcggcttggaagagccactagaggcgtttggcggctactagagaagccgtctgaaagcttttgaagctgttcaggggctaaagaagagccaaccaaggctgtcgtagagattgctttgaagcctgaggaacttacaggtagcttgaactgaggccatccgaacgaaatataaaagtccgatagtatcaattaggatgtatgaattgagttattatacctaatctaattaattgggtagttaactaaagtagtagttttaagctgtaagttgtttgtaggcatgagttattgggtagtccgatatcaatgcactgatagatgacactaggcatgcacttcaatcgctcagtactaggtccagcacagtgaataaccggcttgtgcatagggaataggtttaggtcagaagagttgagaagatcggatgggcagagaaaaaaataatgaaaactttcttaagtttagttttgtctatgttttattgtgccactctgtgacactgtacgatcagagacctttcgtaattaagcgcgtaggtgtctgcagcagtcgaagtagtgggtgaaaaaggatgtattggtacagcgtaccgaaacaactactgtctggtggtgttcgaacggaaaccaacacggattgtctctctctctctgtctggctatcggaggagtaagtgataccgctaggctagcggtcgaagcgacgacaataaacgcaaggactacgaacgcgagtgcccttctgacgtccgggccgcgtcgagtacgtaaccgcagatgacccctgttttgtggctgctcccttcgaatcgtctacgaacacggtgtccgttgatgcttatgcacttgtagcggtgggaaaagttcaaaccgtacccggtttgaggtctctagttggagcggccggtgacactgggagccttgttgccgacgcgcgcgaactcgtcgctttaggtcaggacggtcctcgcaccgcagttccactacgagaggtagcctccaccgcccagaataccggactcaaggtggcttctgcggttcctgcatcgcagtatggagaccgtgtcaaagaacccaccaagacgacgagcgatgagctctggggagcccctacgccaagacctaccgtatctgaagttgtagcggcctccctcgtgtcttgtgatactacttgaacggcgaaaggagttccggatgttgctcgtcctccggccttgcttctttcaagtgaactcacgagcaggcgtcacaggaaagggcctaatgaccgaaccgttgcgtgagtcttgtctagagaagctgaagcacacccacgtcaagaagttgttgggaagcacggtaaagagggtcttgcgatagttagaacgtttacgcaagttgttaacccagtacaggtagggacgcgttttcgacaaggaacccgaaggacgacgaggactccgacgaggttgaccaccgatgtaaggtggggtactagagtatagatttcaagaaggctaggatttcctaaggctgttcatgcgtccttagtacgacacctgatctatggtgctgtctttgaggccgatgtcaagagttcagttcagggtgcacacaggtcgcgcagccaagaggttgtagaatagatacggccacttcagaaggttcatttggacttgccgcatctactagccaccagcttttgaggctagtagtacccaggggtaggcataggcacgcaacgatgcaataccacaaagggaacatacaaccagaaaagttattatattattccccaatcaaaatgcaaaggtataaaaggtacaagcttttgtcatataaacgacggggaaggtttaaactttttctattttatttatatattgatttttataggagaaaaaaaaaagaaagctgtttatatattgagaattgaaagggttaacaaattcgttttctatatttaggagaaggtgtgttttctgcttaggtactaacgacctaacgacagatgaccacggctttaccgctgctctcttcgaacacgatggacgttaatgttcaagcagttgtaacagaaggaacggtacaaaccactgcggtatgagggcactagtcctgtgtggagaccttgtcaaagaacccttcaattagaagaagagccgaggagccgctggttagaacactccaagaagaggacttaccacaggtgaagctgtagcttccagatggactcgcgtntaggtgtcaaggctgatgcacacccacgtcaagatgatgtgtccgttgagcgtctacgggccattgttacccaagaggtaggacgtaccttccacaagggacctgaaggacgacgaggagtccgacgaccttcctcgaggtaaggtgatcactagaatgtgcacagaatagtagttcttaatatcgttcatggctccctaataattttatttttttttcccttcttacccttaatcttaattttgactttggccggtacttcttgcaaagctcacttctgtttgctgtcatactctgccatcaaacgatacctgtacctagcaagggtttcgtcaggttcagaaatacttggccagatagccaagtcggaagttcttggcgctcctattggccgggttctctttgttgtttaacaccactcgaaaatnatatttggcttgccacggcaggcagtctacaatttacctgccgcctatctagaggtctcatttagactccttttagcaaggccggggggatggtgctgggtgcgctaggcaggagagggggtgggggatgtggaaaaagaagaaggcgaggacgctagccaataaactaaaacacatactataggttaaagaaaagacctcaccataggataagattaaagaatctaacaacataacttggtagtcaaaaccaaattcgcgtactaccgcctctcaaagccctctaccctcagtctagggaacaaaagacgacggcttcaccacgttctaagccggctatccaaaaaagagagtaaaattcgagttaatacgccagtaagaacaatccgaaacctcttaaacgagataaagctttctttaacgacgaaagatcaaaactaatcagggatattttaaacgaaagccaagacttataggctcttacagcatagcagttactgctaagaaaaaatcttaagattatgaaacaggacaaaagacactaaattacctcttttataacaaggaaaatcactagatacgagagggctggtaatcctactcccaacttccacttttatgaaagaccattaaaaggagagatttaagaaggtttgtgctgtgttcatattaatatctggttctaactaagaagaatacgtggctaagagtgaagggaagggagacacaataccaatagcaacaatgactaccaacgaattgagtaccccatcgcggacccactaggcaactggacgtccagctg

SEQ ID NO: 29 - protein, 1568 aa, Musa acuminata
  VARIANT 1180, 1313: Xaa = Any Amino Acid
GSQLLGMDLKILVISSKLEKSLPRALSPLMTSVKRCTCLRWTHLVSFGKVRKSAEYFWILSLGWCLHEPQESSKYQKPNHKLKCDIHFCLMHKTGHSPLCLKQKHSSPIHPIRSSEEKIFEIHFRQTKARNPWKGRSSYEFSNTCNSTHTVDVNDPHALSLLGIKPNMRVSLALIPIVRIRVALREGGSELVQWIKTYKFKNEFVNTRRFRFNSNLSRKPKVNSSVNNNFGKVRTHTFKEHTNLKWFGQNDLHPLVKPSSKRLPTSTSKSLRGRTNTSLTTFYNGSYSYKFSTRKKEVNMQAIENKTCRLCGFFSQSIASQKLYSLLRIEGYLTPRGFKFGLQISNALGFPRLPVPPPVSVHWTVYRCHRRTSRVLGGATATFSAHWLDSKLDPNQSELGSNPVTGLDPLILTLIICKLRNKYSPKQVFNRQTSSLLPAIFRQTSDIPLDFFRTPSRVPILWRVRVAEPSRSPQTADDLFGRLSKTSTSPRFLLGWFRQHLRNFGLLECPSNLTPVGLLYIFRLSLILHTLNNMDINPINFHHQNSTFNKHPYSITHQAIVTLSTVITRSRVMIQVVSLIGRTRIPYPNPVFSTLLAYPSLFLLLLKEFKSKQIQNNTVRHCDMLVSGKHFAHPQTSSASSPTFSYITMSHGFVDDRPPQACLWLCLTERERQTDSLLIHYGDPIASFAAVICVPDACAHGKTAGPAQLMHWRLLGTKHRRGKLSRCLCHRQLRIREHRHPFQVWHGPNSRDQPRRPLPSEQRLRALEQRNPVLPGAWRQGDALHRRWRVLWPEFHRRRQGRSVIPLAQFLGWFCCSLLETPRGCGSGWHRLQHRRREHRTLTCRFPQGLQRAGGRNEESSLECSSAVSFPGLLAWQRTQNRSLRLRVGAVLQQPFVPFLPERYQSCKCVQQLGHVHPCAKAVPWASCCSGCSNWWLHSTPSHISSSDPKGFRQVRRNHAVDIPRQKLRLQFSSQVPRVSSASVLQHLIYAGEVFQVNLNGVDDRWSKTPIIMGPHPYPCVATLWCFPCMLVFSIIGVSFTFPYFPCSKTVYLLPLPNLKKIKIYNKYPLFFFFRQIYNSLSQLFKQKIILFHTKDESMIAGLLSTGAEMATREACATCNYKFVNIVFLAMFGDAILPSGHTSGTVSWEVNLLLGSSATNLVRFFSMVSTSTSKVYLSAXPQFRLRVGAVLLHRQLADARQWVLHPAWKVFPGLPAAPQAAGRSSIPLVILHVSYHQELQVPRDYNKKKGKNGNNNNRPRTFRVKTNDSMRRFAMDMDRSQSSPSLYEPVYRFSLQEPRGPAQEKQQIVVSFXYKPNGAVRQMLNGRRIDLQSKSEENRSGPPTTTHAIRPLPHPLHLFLLPLLRSVIFCVYPISFLEWYPILISIVVLNHQFWFKRMMAESFGRWESDPLFSAAEVVQDSADRFFLSFAQLCGHSCALENLLYFERNCCFLVLISPYKICFRFISENVVSSMTILFNSNTLSCFLFNGENIVPFSDLCSPDHDEGRKYFLVIFLSKFFQTRHKYNYRPRLILLMHRFSLPFPLCYGYRCYWLLNSWGSAWVIRPAGR

SEQ ID NO: 30 - protein, 1574 aa, Musa acuminata
  VARIANT 158, 271, 1179, 1317: Xaa = Any Amino Acid
DPNFEWILKFLVQSKNLYQELVHHPNGVHVSDGLTWFHSEKFERVHKNIDFGFFHSVGAFMSDLKSPPNIKSRITNNVIEFIFVCTKQGIHSLCVSKNILLRFIPFAHRKRKFLKSIFDNRPKLEIHGNEEDPHMSFPIHVIRLIKHRWMCNEMTLMXYLSWVLNQIEALLYQLLGSEWHERGGVNCSGLKLISLKMNSIREDFVLIVTVDENQKLTVVITISGKELTHSRNIPISGSVKMTYIHLSLLRRGSQLPLANHFEGEGQIPLLXPFTMVHTLTNFQRERRRTCKQLKTRLAKDFAKAFFLNLLLLKSCILCELRGIYRPQEDLNLGSKFRMLLGSRGCRCHRLSVFDTGQCTSGATAGPLGCWAVPPPRLFQLTGWIPNLTQTSPNSGPIDPPDYRINPSPLYANYATENIVLSKFLTGKRRVFFRRSFGRLLIYLWISSSGLLVGSRSCGEFSEPNLLGDLRKPPMISSADFRKLRQVPDFFSVGSDSISNETSDSLNVHRTLRACFIFSGYHSSYILNSIIWIRLINPSIDFIIKIRHSTNIRTQPIRLLRDYLLSVREVSESSRSCHLLAEHVSLIQIQSSQLFPTRLFFYYFKNSNQNRYKITRDTVTCSLESINSRIHRRRQLHHPLFPTPCRMALLMTDHHKLAFGCAQRERDRPIASSFTMAIRSPASLLLFAFLMLALTGRLQARRSSCIGVYWGQNTDEGSLADACATGNYEYVNIATLFKFGMGQTPEINLAGHCDPRNNGCARLSSEIQSCQERGVKVMLSIGGGGSYGLSSTEDAKDVASYLWHSFLGGSAARYSRPLGDAVLDGIDFNIAGGSTEHYDELAAFLKAYNEQEAGTKKVHLSARPQCPFPDYWLGNALRTDLFDFVWVQFFNNPSCHFSQNAINLANAFNNWVMSIPAQKLFLGLPAAPEAAPTGGYIPPHDLISKVLPILKDSDKYAGIMLWTRYHDRNSGYSSQVKSHVCPARRFSNILSMPVKSSKTTAMIGGRKLRSSWVPIRIRALLRYGVSLVCWSFQYNKGLVLRFHIFHVRKQYICCPFQIKRNKYITKNILFFFSFDKYITLNFPNCLSKRYKSSSTQKTNPLLDCCLLVPKWRREKLVLPAITSSSTLSSLPCLVTPYSRDQDTPLEQFLGKLIFFSAPRRPILGSSPEWCPLRHRRSTAXIHSSDYVWVQFYYTGNSQMPGNNGFSILHGRCSLDFLLLLRLLEGAPFHSYTCLIIKNYSKYRGIIKIKKKGRMGIRIKTETGHEERFERQTTVDGSLLWTWIVPKAVQVFMNRSIGSAFKNREDNRPKRNNKLWAFXINRTVPSVRCMDGGISRVNLRKIVPAPLPRPTRSVLSPTPYTFFFFRSCDRLFDFVYDIQFLFWSGILFFLRLLYTISFGLSAWRRVSGDGSQIPCFLLPKWCKIRPIGFFSHFKLNYAVILVRLWRICSISKEIAAFFLVPIKFAFGSEYPRMSYRQRFFFRILILCPVFCDLMEKILFLLVIYALPTIRMRVEGENTFWFSSLNSSKHDTSIIIDQDFFLCTDSHFPSLCVMVIVVTDGCLTHGVAPGSVDLQVD

SEQ ID NO: 31 - protein, 1562 aa, Musa acuminata
  VARIANT 163, 271, 1170, 1311: Xaa = Any Amino Acid
RIPTFRNGSNFSYKFKVRKIFTKSFESIDDIRETVYMSPMDSLGFIRKSSKECIRILILDSFTRLVPSVTSRVLQISKAESQIEMLNSFLSNAQNRAFIAFVFKAKTFFSDSSHSLIGRENFNPFSTIDQSSKSMEMRKILIVFQYMFDSLNIGGCVMKPSCXISLGYTKYESEPCSDTNCDQSGTKRGGEISAVDNLVKIRKYEKISFLEMKTKSQCKQFRESKNSHIQGTYQFKVVRSKPTSTCEAFFEEAPNFHQITLKGKDKYLSYXLLQWFILLQIFNEKEGGEHASNKQDLLKTLLRLFFSIYCFSKVVFSAENGVFIDPKRIIWAPNFECSWVPEVAGATACQCLTLDSVLAVPPPDLSGVGRCHRLDFFSSLVGFQTPKPVRTRVQLTRNRIIGLTLNPNPNYMQTTQLKISASFPANVESSSGDLSADFYTFGFLLADSGPDLVASLASSRTFSVISANRRSLRQTFENFDKSPISSRLVPTASLTKLRTPMSIELDSGRLALYFQAIIVNPTYLTQYGLDLTHQLISSSKFDIQQTSVLNNPSGYSYVTIYCDPYVKLASHDPGRVTYWPNTYPLSKSSLLNSSSLPVSFFITFERIQIKTDTKHGETLHASLWKALIRASTDVVSFITHFFLHNHVAWLCQTTTSLPLVVPNRERETDRPPHSLWRSDRQLRCCYLRSCLRSREDCRPGAAHALASTGKTPTREAQMLVPQATTNTTSPPFSSLAWAKLQRSTSPATVTLGTTAARAAAKSSPARSVASRCSPSEVAGLMAVPPKTPRTRHTSGTVSWVVLLLATRDPSGMRFWMATSTSPEGAQNTMMNLPLSSRPTTSRRPERRKFTVLVRSVLSRITGLATHSEQISSTSCGCSSSTTLRAISPRTLSILQMRSTIGSCPSLRKSCSLGFLLLLRLLQLVATFHPMISYLKFFRSRIPTSTQESCCGLDTTTETPATVLKSSPTCVQRVGSPTSYLCRSLPSKPERRRSVVENSDHHGSPSVSVRCYVMVFPLYVGLFNNIIRGFYVSIFSMFENSIFAAPSKFEKDKINILKISSFFFLSTNILLTFPIVAKDINPLPHKRRIHDCWIAVYWCRNGDERSLCYLQLQVRQHCLPCHVWRHTPVIRTHLWNSFLGSSSSRLLGDQSCEVLLLNGVHFDIEGLPERXSTVPTTCGCSSTTQATRRCPVTMGSPSCMEGVPWTSCCSSGCWKELHSTSDLTRVLSSRIIASTEGLLKKKREEWELELKLKPAMKNVSSEDKRQYETVVCYGHGSFPKQSKSLTGLSVQPSRTARITGPRETTNCGELLXTERCRPSDVKWTADRSPEIGKSFRPPYHDPRDPSSPPPPTPFSSSAPAIGYLILCMISNFFSGVVSYSNFLDCCIEPSVLVAHDGGEFREMGVRSLVFCCRSGARFGRVFSLILSSIMRSFLLGFGEFALFRKKLLLSSFDSLNLLSVLNIRECRIVNDDSFLEFYFVLFSVIWRKYCSFSMLSRPLGGLKVKILSGNFPLILPNTTQVLTKIDSSYAPILTSLPSVLWLSLLLMVALMGRLGDPLTCRST

SEQ ID NO: 32 - DNA, 2392 bp, Musa acuminata
  misc_feature 1721: n = A,T,C or G
tcactggtacggggcccccctcgaggtcgacggtatcgataagctttgatctcttctctcaatctctctctctctctctctctctctctctctctgtatgtctttaaatatggttgtaatgctgaattgctatgtttatcttggccaaactgtgtccatctttgagcagataaatctggcgataatgttctttttactgaaagcactgcaggatgagggcctgaaatcacatcggacgcccactgggtcatgatgatatggactcctccacagcgagcagccatgggatgtgagatccacatagcagcgtagataagggaagcccgcaacactaggctgttgttgttccagtaaagatcgaaaggtcaggcgacagtgacgatcgactttttcgagcatgatgacaacgacgacctgctcctgcaatatccgtcccctaccgtagagtgggaataaatgggtttgtagttgcactatttctcgcaggaattaattgaaagccctgcaaattgctgtttctctttccttatattaaaccttcctcctgttacattaaaattgcatgttaagacatttctgtatggatccgaacatgagatctatcattgaagtaatgggtaggatttacattatcatcatcatcatcatctccatgggtttggatctaattagaccgaaaacctcatttaaaatccaaccccaatattggcttgacttgctccatctccaagaaaaatacaacaagaacaacaaaaatttaggatgcacattgaattgatttggtcactatgagagaatcatggattaaaaatattaaaataaaaaataaatcataatcatctactcactctaacgattcacattctatccaccaaatttgacatcggcttctaattaatttcatatattaggttctaaaaaatctctccctttgacagatgaataaatatttcttttaattcgttagggaaggatctaatataatatatatatatatatatatttatttattagattctaaccatttctctcaccagaatatgaatcgacggccatatctgcaaaaacccaccaattgttcacagtaaacgctcattgaattaaggtcgaaattacttttaaatttctagagatttccaataaaatatactcgtatcttttacagtgatgatgctccggatgataagatggaaggatgcgtgtgtcagccgcctgcgatctctgtggcggggacgagacgaagacaaggacgtgagcggacgataccaagtcttctcctcccccaccacgcacgtctcagattcccgatacggcctatcccggtggcgtgtggactgcacagacgaacgagtaaatgcccatcccccctctttcattctttctctttgcgtgtgtgagaggagcgcctataaataagcacgaaacaagccccttttctctccaagaacacaccacaccattcacacactacatcctctgcttcttcgagccttttcgccttccttcctcgtctaaccatgtcgacctgcggcaactgcgactgcgttgacaagagccagtgcgtgtaagtcatcctccatccctccacctcttcttcttcttcttcttcttcttcttctaacctcgccccgtttgtgtttgatgagtcgactcttcccacatcgctcgtcaaaactcagagctttattagggaacatcagcaatactatatgtatatgtanaaggtcaacgttggctgaagaacttggttttgcctttgcaggaagaaaggaaacagctacggtatcgatattgttgagaccgagaagaggtactgattagcttcttctccctcctcctcgtcgaggatgatcaaactaattaggattacaccttattaccttacctaatgctttttccgtattcgtttcgtctcttcagctacgtcgacgaggtgatcgttgccgcagaagctgccgagcatgacggcaagtgcaagtgcggcgccgcctgcgcctgcaccgactgcaagtgtggcaactgagaagcacttgtgtcactaccactaaataaaagtttgcaatgcataaaaaacaaaagaacaaaaaaaaaaaaggaagaagaagaaggtgtggctatgtactctaataattcgggcaggctgataggttgtaagatgggataacgcagtatcatctgtgttatctctgtcctgtgttacaactctcctatctatcctagtcaatgaaatattattagtattaatctggttgtgtcattcatatatgctgctgctgctgctgcttcctctttcaccaatcaacccaaaggatcgattgcactgtaaggcccaacttcctcaccgatatgctcgctcagttacgatgaatgaacagcaaccaaacgagtctgc

SEQ ID NO: 33 - DNA, 2392 bp, Musa acuminata
  misc_feature 1721: n = A,T,C or G
agtgaccatgccccggggggagctccagctgccatagctattcgaaactagagaagagagttagagagagagagagagagagagagagagagagacatacagaaatttataccaacattacgacttaacgatacaaatagaaccggtttgacacaggtagaaactcgtctatttagaccgctattacaagaaaaatgactttcgtgacgtcctactcccggactttagtgtagcctgcgggtgacccagtactactatacctgaggaggtgtcgctcgtcggtaccctacactctaggtgtatcgtcgcatctattcccttcgggcgttgtgatccgacaacaacaaggtcatttctagctttccagtccgctgtcactgctagctgaaaaagctcgtactactgttgctgctggacgaggacgttataggcaggggatggcatctcacccttatttacccaaacatcaacgtgataaagagcgtccttaattaactttcgggacgtttaacgacaaagagaaaggaatataatttggaaggaggacaatgtaattttaacgtacaattctgtaaagacatacctaggcttgtactctagatagtaacttcattacccatcctaaatgtaatagtagtagtagtagtagaggtacccaaacctagattaatctggcttttggagtaaattttaggttggggttataaccgaactgaacgaggtagaggttctttttatgttgttcttgttgtttttaaatcctacgtgtaacttaactaaaccagtgatactctcttagtacctaatttttataattttattttttatttagtattagtagatgagtgagattgctaagtgtaagataggtggtttaaactgtagccgaagattaattaaagtatataatccaagattttttagagagggaaactgtctacttatttataaagaaaattaagcaatcccttcctagattatattatatatatatatatatataaataaataatctaagattggtaaagagagtggtcttatacttagctgccggtatagacgtttttgggtggttaacaggtgtcatttgcgagtaacttaattccagctttaatgaaaatttaaagatctctaaaggttattttatatgagcatagaaaatgtcactactacgaggcctactattctaccttcctacgcacacagtcggcggacgctagagacaccgcccctgctctgcttctgttcctgcactcgcctgctatggttcagaagaggagggggtggtgcgtgcagagtctaagggctatgccggatagggccaccgcacacctgacgtgtctgcttgctcatttacgggtaggggggagaaagtaagaaagagaaacgcacacactctcctcgcggatatttattcgtgctttgttcggggaaaagagaggttcttgtgtggtgtggtaagtgtgtgatgtaggagacgaagaagctcggaaaagcggaaggaaggagcagattggtacagctggacgccgttgacgctgacgcaactgttctcggtcacgcacattcagtaggaggtagggaggtggagaagaagaagaagaagaagaagaagaagattggagcggggcaaacacaaactactcagctgagaagggtgtagcgagcagttttgagtctcgaaataatcccttgtagtcgttatgatatacatatacatnttccagttgcaaccgacttcttgaaccaaaacggaaacgtccttctttcctttgtcgatgccatagctataacaactctggctcttctccatgactaatcgaagaagagggaggaggagcagctcctactagtttgattaatcctaatgtggaataatggaatggattacgaaaaaggcataagcaaagcagagaagtcgatgcagctgctccactagcaacggcgtcttcgacggctcgtactgccgttcacgttcacgccgcggcggacgcggacgtggctgacgttcacaccgttgactcttcgtgaacacagtgatggtgatttattttcaaacgttacgtattttttgttttcttgttttttttttttccttcttcttcttccacaccgatacatgagattattaagcccgtccgactatccaacattctaccctattgcgtcatagtagacacaatagagacaggacacaatgttgagaggatagataggatcagttactttataataatcataattagaccaacacagtaagtatatacgacgacgacgacgacgaaggagaaagtggttagttgggtttcctagctaacgtgacattccgggttgaaggagtggctatacgagcgagtcaatgctacttacttgtcgttggtttgctcagacg

SEQ ID NO: 34 - protein, 758 aa, Musa acuminata
  VARIANT 548: Xaa = Any Amino Acid
SLVRGPPRGRRYRALISSLNLSLSLSLSLSLYVFKYGCNAELLCLSWPNCVHLADKSGDNVLFTESTAGGPEITSDAHWVMMIWTPPQRAAMGCEIHIAAIREARNTRLLLFQRSKGQATVTIDFFEHDDNDDLLLQYPSPTVEWEMGLLHYFSQELIESPANCCFSFLILNLPPVTLKLHVKTFLYGSEHEIYHSNGDLHYHHHHHLHGFGSNTENLINPTPILALAPSPRKIQQEQQKFRMHIELIWSLENHGLKILKINHNHLLTLTIHILSTKFDIGFLISYIRFKISPFDRINISFNSLGKDLIYIYIYIFIYILTISLTRIIDGHICKNPPIVHSKRSLNGRNYFISRDFQNILVSFTVMMLRMIRWKDACVSRLRSLWRGRDEDKDVSGRYQVFSSPTTHVSDSRYGLSRWRVDCTDERVNAHPPSFILSLCVCERSAYKARNKPLFSPRTHHTIHTLHPLLLRAFSPSFLVPCRPAATATALTRASACKSSSIPPPLLLLLLLLLLTSPRLCLMSRLFPHRSSKLRALLGNISNTICICXRSTLAEELGFAFAGRKETATVSILLRPRRGTDLLLPPPRRGSNLGLHLITLPNAFSVFVSSLQLRRRGDRCRRSCRARQVQVRRRLRLHRLQVWQLRSTCVTTTKKFAMHKKQKNKKKKGRRRRCYVLFGQADRLDGITQYHLCYLCPVLQLSYLSSMKYYYSGCVIHICCCCCCFLFHQSTQRIDCTVRPNFLTDMLAQLRMNSNQTSL

SEQ ID NO: 35 - protein, 758 aa, Musa acuminata
  VARIANT 541: Xaa = Any Amino Acid
HWYGAPLEVDGIDKLSLLSISLSLSLSLSLCMSLNMVVMLNCYVYLGQTVSIFEQINLAIMFFLLKALQDEGLKSHRTPTGSYGLLHSEQPWDVRSTQRRGKPATLGCCCSSKDRKVRRQRSTFSSMMTTTTCSCNIRPLPSGNKWVCSCTISRRNLKALQIAVSLSLYTFLLLHNCMLRHFCMDPNMRSIIEVMGRIYIIIIIIISMGLDLIRPKTSFKIQPQYWLDLLHLQEKYNKNNKNLGCTLNFGHYERIMDKYNKKIIIIYSLRFTFYPPNLTSASNFHILGSKKSLPLTDEIFLLIRGRIYNIYIYIYLFIRFPFLSPEYESTAISAKTHQLFTVNAHIKVEITFKFLEISNKIYSYLLQCSGDGRMRVSAACDLCGGDETKTRTADDTKSSPPPPRTSQIPDTAYPGGVWTAQTNEMPIPPLSFFLFACVRGAPINKHETSPFSLQEHTTPFTHYILCFFEPFRLPSSSNHVDLRQLRLRQEPVRVSHPPSLHLFFFFFFFFPRPVCVVDSSHIARQNSELYGTSAILYVYVXGQRWLKNLVLPLQEERKQLRYRYCDREEVLISFFSLLLVEDDQTNDYTLLPYLMLFPVSFRLFSYVDEVIVAAEAAEHDGKCKCGAACACTDCKCGNEALVSLPLNNKSLQCIKNKRTKKKKEEEEGVAMYSNNSGRLIGCKMGRSIICVISVLCYNSPIYPSQNIISINLVVSFIYAAAAAASSFTNQPKGSIALGPTSSPICSLSYDETATKRVC

SEQ ID NO: 36 - protein, 762 aa, Musa acuminata
  VARIANT 546: Xaa = Any Amino Acid
LTGTGPPSRSTVSISFDLFSQSLSLSLSLSLSVCLIWLCIAMFILAKLCPSLSRIWRCSFYKHCRMRANHIGRPLGHDDMDSSTASSHGMDPHSSVDKGSPQHAVVVPVKIERSGDSDDRLFRAQRRPAPAISVPYRRVGINGFVVALFLAGINKPCKLLFLFPYIKPSSCYIKIACDISVWIRTDLSLKWVGFTLSSSSSSPWVWILDRKPHLKSNPNIGLTCSISKKNTTRTTKIDAHIDLVTMRESWIKNIKIKNKSSSTHSNDSHSIHQIHRLLINFIYVLKNLSLQMNKYFFFVREGSNIIYIYIYIYLLDSNHFSHQNMNRRPYLQKPTNCSQTLIELRSKLLLNFRFPIKYTRIFYSDDAPDDKMEGCVCQPPAISVAGTRRRQGRERTIPSLLLPHHARLRFPIRPIPVACGLHRRTSKCPSPLFHSFSLRVEERLISTKQAPFLSKNTPHHSHTTSSASSSLFAFLPRLTMSTCGNCDCVDKSQCVVILHPSTSSSSSSSSSSNLAPFVFDESTLPTSLVKTQSFIREHQQYYMYMXKVNVGRTWFCLCRKKGNSYGIDIVETEKRYLASSPSSSSRMIKLIRITPYYLTCFFRIRFVSSATSTRSLPQKLPSMTASASAAPPAPAPTASVATEKHLCHYHIKVCNAKTKEQKKKRKKKKVWLCTLIIRAGVVRWDNAVSSVLSLSCVTTLLSILVNEILLVLIWLCHSYMLLLLLLPLSPINPKDRLHCKAQLPHRYARSVTMNEQQPNESA

SEQ ID NO: 37 - DNA, 1880 bp, Musa acuminata
  misc_feature 1721, 1782, 1788, 1799: n = A,T,C or G
tcactggtacggggcccccctcgaggtcgacggtatcgataagctttgatctcttctctcaatctctctctctctctctctctctctctctctctgtatgtctttaaatatggttgtaatgctgaattgctatgtttatcttggccaaactgtgtccatctttgagcagataaatctggcgataatgttctttttactgaaagcactgcaggatgagggcctgaaatcacatcggacgcccactgggtcatgatgatatggactcctccacagcgagcagccatgggatgtgagatccacatagcagcgtagataagggaagcccgcaacactaggctgttgttgttccagtaaagatcgaaaggtcaggcgacagtgacgatcgactttttcgagcatgatgacaacgacgacctgctcctgcaatatccgtcccctaccgtagagtgggaataaatgggtttgtagttgcactatttctcgcaggaattaattgaaagccctgcaaattgctgtttctctttccttatattaaaccttcctcctgttacattaaaattgcatgttaagacatttctgtatggatccgaacatgagatctatcattgaagtaatgggtaggatttacattatcatcatcatcatcatctccatgggtttggatctaattagaccgaaaacctcatttaaaatccaaccccaatattggcttgacttgctccatctccaagaaaaatacaacaagaacaacaaaaatttaggatgcacattgaattgatttggtcactatgagagaatcatggattaaaaatattaaaataaaaaataaatcataatcatctactcactctaacgattcacattctatccaccaaatttgacatcggcttctaattaatttcatatattaggttctaaaaaatctctccctttgacagatgaataaatatttcttttaattcgttagggaaggatctaatataatatatatatatatatatatttatttattagattctaaccatttctctcaccagaatatgaatcgacggccatatctgcaaaaacccaccaattgttcacagtaaacgctcattgaattaaggtcgaaattacttttaaatttctagagatttccaataaaatatactcgtatcttttacagtgatgatgctccggatgataagatggaaggatgcgtgtgtcagccgcctgcgatctctgtggcggggacgagacgaagacaaggacgtgagcggacgataccaagtcttctcctcccccaccacgcacgtctcagattcccgatacggcctatcccggtggcgtgtggactgcacagacgaacgagtaaatgcccatcccccctctttcattctttctctttgcgtgtgtgagaggagcgcctataaataagcacgaaacaagccccttttctctccaagaacacaccacaccattcacacactacatcctctgcttcttcgagccttttcgccttccttcctcgtctaaccatgtcgacctgcggcaactgcgactgcgttgacaagagccagtgcgtgtaagtcatcctccatccctccacctcttcttcttcttcttcttcttcttcttctaacctcgccccgtttgtgtttgatgagtcgactcttcccacatcgctcgtcaaaactcagagctttattagggaacatcagcaatactatatgtatatgtanaaggtcaacgttggctgaagaacttggttttgcctttgcaggaagaaaggaaacagctacngtatcnatattgttganaccgagaagaggtactgattagcttcttctccctcctcctcgtcgaggatgatcaaactaattaggattacaccttattac

SEQ ID NO: 38 - DNA, 1878 bp, Musa acuminata
  misc_feature 1720, 1768, 1781, 1787, 1798, 1807, 1820, 1845, 1869: n = A,T,C or G
agtgaccatgccccggggggagctccagctgccatagctattcgaaactagagaagagagttagagagagagagagagagagagagagagagagacatactgaaatttataccaacattacgacttaacgatacaaatagaaccggtttgacacaggtagaaactcgtctatttagaccgctattacaagaaaaatgactttcgtgacgtcctactcccggactttagtgtagcctgcgggtgacccagtactactatacctgaggaggtgtcgctcgtcggtaccctacactctaggtgtatcgtcgcatctattcccttcgggcgttgtgatccgacaacaacaaggtcatttctagctttccagtccgctgtcactgctagctgaaaaagctcgtactactgttgctgctggacgaggacgttataggcaggggatggcatctcacccttatttacccaaacatcaacgtgataaagagcgtccttaattaactttcgggacgtttaacgacaaagagaaaggaatataatttggaaggaggacaatgtaattttaacgtacaattctgtaaagacatacctaggcttgtactctagatagtaacttcattacccatcctaaatgtaatagtagtagtagtagtagaggtacccaaacctagattaatctggcttttggagtaaattttaggttgggttataaccgaactgaacgaggtagaggttctttttatgttgttcttgttgtttttaaatcctacgtgtaacttaactaaaccagtgatactctcttagtgcctaatttttataattttattttttatttagtattagtagatgagtgagattgctaagtgtaagataggtggtttaaactgtagccgaagattaattaaagtatataatccaagattttttagagagggaaactgtctacttatttataaagaaaattaagcaatcccttcctagattatattatatatatatatatatataaataaataatctaagattggtaaagagagtggtcttatacttagctgccggtatagacgtttttgggtggttaacaagtgtcatttgcgagtaacttatctccagctttaatgaaaatttaaagatctctaaaggttattttatatgagcatagaaaatgtcactactacgaggcctactattctaccttcctacgcacacagtcggcggacgctagagacaccgcccctgctctgcttctgttcctgcactcgcctgctatggttcagaagaggagggggtggtgcgtgcagagtctaagggctatgccggatagggccaccgcacacctgacgtgtctgcttgctcatttacgggtaggggggagaaagtaagaaagagaaacgcacacactctcctcgcggatatttattcgtgctttgttcggggaaaagagaggttcttgtgtggtgtggtaagtgtgtgatgtaggagacgaagaagctcggaaaagcggaaggaaggagcagattggtacagctggacgccgttgacgctgacgcaactgttctcggtcacgcacattcagtaggaggtagggaggtggagaagaagaagaagaagaagaagaagaagattggagcggggcaaacacaaactactcagctgagaagggtgtagcgagcagttttgagtctcgaaataatcccttgtagtcgttatgatatacatatacatnttccagttgcaaccgacttcttgaaccaaaacggaaacgtccttcttncctttgtcgatgncatagntataacaactntggcttttntccatgactaatngaagaagagggaggaggagcagctntactagtttgattaatcctaatgnggaataatg

SEQ ID NO: 39 - protein, 597 aa, Musa acuminata
  VARIANT 546, 562, 572, 575, 579, 588, 594: Xaa = Any Amino Acid
SLVRGPPRGRRYRALISSLNLSLSLSLSLSLYFKYGCNAELLCLSWPNCVHLADKSGDNVLFTESTAGGPEITSDAHWVMMIWTPPQRAAMGCEIHAAIREARNTRLLLFQRSKGQATVTIDFFEHDDNDDLLLQYPSPTVEWEMGLLHYFSQELIESPANCCFSFLILNLPPVTLKLHVKTFLYGSEHEIYHSNGDLHYHHHHHLHGFGSNTENLINPTILALAPSPRKIQQEQQKFRMHIELIWSLENHGLKILKKINHNHLLTLTIHILSTKFDIGFLISYIRFKISPFDRINISFNSLGKDLIYIYIYIFIYILTISLTRIIDGHICKNPPIVHSKRSLNGRNYFISRDFQNILVSFTVMMLRMIRWKDACVSRLRSLWRGRDEDKDVSGRYQVFSSPTTHVSDSRYGLSRWRVDCTDERVNAHPPSFILSLCVCERSAYKARNKPLFSPRTHHTIHTLHPLLLRAFSPSFLVPCRPAATATALTRASACKSSSIPPPLLLLLLLLLLTSPRLCLMSRLFPHRSSKLRALLGNISNTICICXRSTLAEELGFAFAGRXETATVSILLXPKXGTDXLLLPPPRRXSNLGLXLIT

SEQ ID NO: 40 - protein, 590 aa, Musa acuminata
  VARIANT 540, 556, 560, 562, 565, 568, 572, 580, 588: Xaa = Any Amino Acid
HWYGAPLEVDGIDKLSLLSISLSLSLSLSLCMSLNMVVMLNCYVYLGQTVSIFEQINLAIMFFLLKALQDEGLKSHRTPTGSYGLLHSEQPWDVRSTQRRGKPATLGCCCSSKDRKVRRQRSTFSSMMTTTTCSCNIRPLPSGNKWVCSCTISRRNLKALQIAVSLSLYTFLLLHNCMLRHFCMDPNMRSIIEVMGRIYIIIIIIISMGLDLIRPKTSFKIQPYWLDLLHLQEKYNKNNKNLGCTLNFGHYERIDKYNKKIIIIYSLRFTFYPPNLTSASNFHILGSKKSLPLTDEIFLLIRGRIYNIYIYIYLFIRFPFLSPEYESTAISAKTHQLFTVNAHIKVEITFKFLEISNKIYSYLLQCSGDGRMRVSAACDLCGGDETKTRTADDTKSSPPPPRTSQIPDTAYPGGVWTAQTNEMPIPPLSFFLFACVRGAPINKHETSPFSLQEHTTPFTHYILCFFEPFRLPSSSNHVDLRQLRLRQEPVRVSHPPSLHLFFFFFFFFFPRPVCVVDSSHIRAQNSELYGTSAILYVYVXGQRWLKNLVLPLQEEXKQLXYXYCXRKXVLIXFFSLLLVXDDQTNDYXLL

SEQ ID NO: 41 - protein, 441 aa, Musa acuminata
TGTGPPSRSTVSISFDLFSQSLSLSLSLSLSVCLIWLCIAMFILAKLCPSLSRIWRCSFYKHCRMRANHIGRPLGHDDMDSSTASSHGMDPHSSVDKGSPQHAVVVPVKIERSGDSDDRLFRAQRRPAPAISVPYRRVGINGFVVALFLAGINKPCKLLFLFPYIKPSSCYIKIACDISVSWIRTDLSLKWVGFTLSSSSSSPWVWILDRKPHLKSNPNIGLTCSISKKNTTRTTKIDAHIDLVTMRESWIKNIKIKNKSSSTHSNDSHSIHQIHRLLINFIYVLKNLSLQMNKYFFFVREGSNIIYIYIYLRSKLLLNFRFPIKYTRIFYSDDAPDDKMEGCVCQPPAISVAGTRRRQGRERTIPSLLLPHHARLRFPIRPIPVACGLHRRTSKCPSPLFHSFSLRVEERLISTKQAPFLSKNTPHHSHTTSSASSSLFA

SEQ ID NO: 42 - DNA, 17 bp, Artificial Sequence
  primer
gatcgccatggtgaatg

SEQ ID NO: 43 - DNA, 17 bp, Artificial Sequence
  primer
gtaaaacgacggccagt

SEQ ID NO: 44 - DNA, 2156 bp, Musa acuminata
  misc_feature 879: n = A,T,C or G
ggatcccaacttttaggaatggatcttaaaattttagttataagttcaaagttagaaaaatctttaccaagagctttgagtccattgatgacatccgtgaaacggtgtacatgtctccgatggactcacttggtttcattcggaaaagttcgaaagagtgcataagaatattgattttggattctttcactcggttggtgccttcatgagtgacctcaagagtcctccaaatatcaaaagccgaatcacaaattgaaatgtgattgaattcatttttgtctaatgcacaaaacagggcattcatagcctttgtgtttaaagcaaaaacattcttctccgattcatcccattcgctcatcggaagagaaaatttttgaaatccattttcgacaatagaccaaagctcgaaatccatggaaatgaggaagatcctcatatgagttttccaatacatgtaattcgactcattaaacataggtggatgtgtaatgaaatgaccctcatgcsctatctctcttgggtattaaaccaaatatgagagtgagccttgctctgataccaattgttaggatcagagtggcactaagagagggggggagtgaattagtgcagtggattaaaacttataagtttaaaaatgaattcgtaaatacgagaagatttcgttttaatagtaacttgagtagatgaaaaccaaaagttaacagtagtgtaaataacaatttcgggaaagtaagaactcacacattcaaggaacataccaatttaaagtggttcggtcaaaatgacctacatccacttgtgaagccttcttcgaagaggctcccaacttccactagcaaatcactttgaaggggaaggacaaatacctctcttacnaccttttacaatggttcatactcttacaaattttcaacgagaaagaaggaggtgaacatgcaagcaattgaaaacaagacttgctaaagactttgctaaggctttttttctcaatctattgcttctcaaaagttgtattctctgctgagaattgaggggtatttatagaccccaagaggatttaaatttgggctccaaatttcgaatgctcttgggttcccgaggttgccggtgccaccgcctgtcagtgtttgacactggacagtgtactagcggtgccaccgccggacctctcgggtgttgggcggtgccaccgcctagactttttcagctcactggttggattccaaacttgacccaaaccagtccgaactcgggtccaattgacccgtaaccggattataggattaacccttaatcctaaccctaattatatgcaaactacgcaactgaaaatatagtcctaagcaagtttttaaccggcaaacgtcgagtcttcttccggcgatctttcggcagacttctgatatacctttggatttcttctagcggactcctagtagggtcccgatcttgtggcgagtttagcgagtagccgaaccttctcggtgatctccgcaaaccgccgatgatctcttcggcagactttcgaaaacttcgacaagtccccgatttcttctcggttggttccgacagcatctctaacgaaacttcggactccttgaatgtccatcgaacttgactccggtaggcttgctttatattttcaggctatcatagttaatcctacatacttaactcaataatatggattagattaattaacccatcaattgatttcatcatcaaaattcgacattcaacaaacatccgtactcaataacccatcaggctatagttacgtgactatctactgtgatccgtacgtgaagttagcgagtcatgatccaggtcgtgtcacttattggccgaacacgtatcccttatccaaatccagtcttctcaactcttctagcctacccgtctctttttttattacttttgaaagaattcaaatcaaaacagatacaaaataacacggtgagacactgtgacatgctagtctctggaaagcattaattcgcgcatccacagacgtcgtcagcttcatcacccactttttcctacataccatgtcgcatggctttgttgatgacagaccaccacaagcttgcctttggttgtgcctaacagagagagagagagagacagaccgatagcctcctcattcactatgg

SEQ ID NO: 45 - DNA, 2160 bp, Musa acuminata
  misc_feature 883: n = A,T,C or G
ggatcccaacttttaggaatggatcttaaaattttagttataagttcaaagttagaaaaatctttaccaagagctttgagtccattgatgacatccgtgaaacggtgtacatgtctccgatggactcacttggtttcattcggaaaagttcgaaagagtgcataagaatattgattttggattctttcactcggttggtgccttcatgagtgacctcaagagtcctccaaatatcaaaagccgaatcacaaattgaaatgtgattgaattcatttttgtctaatgcacaaaacagggcattcatagcctttgtgtttaaagcaaaaacattcttctccgattcatcccattcgctcatcggaagagaaaatttttgaaatccattttcgacaatagaccaaagctcgaaatccatgcatggaaatgaggaagatcctcatatgagttttccaatacatgtaattcgactcattaaacataggtggatgtgtaatgaaatgaccctcatgcsctatctctcttgggtattaaaccaaatatgagagtgagccttgctctgataccaattgttaggatcagagtggcactaagagagggggggagtgaattagtgcagtggattaaaacttataagtttaaaaatgaattcgtaaatacgagaagatttcgttttaatagtaacttgagtagatgaaaaccaaaagttaacagtagtgtaaataacaatttcgggaaagtaagaactcacacattcaaggaacataccaatttaaagtggttcggtcaaaatgacctacatccacttgtgaagccttcttcgaagaggctcccaacttccactagcaaatcactttgaaggggaaggacaaatacctctcttacnaccttttacaatggttcatactcttacaaattttcaacgagaaagaaggaggtgaacatgcaagcaattgaaaacaagacttgctaaagactttgctaaggctttttttctcaatctattgcttctcaaaagttgtattctctgctgagaattgaggggtatttatagaccccaagaggatttaaatttgggctccaaatttcgaatgctcttgggttcccgaggttgccggtgccaccgcctgtcagtgtttgacactggacagtgtactagcggtgccaccgccggacctctcgggtgttgggcggtgccaccgcctagactttttcagctcactggttggattccaaacttgacccaaaccagtccgaactcgggtccaattgacccgtaaccggattataggattaacccttaatcctaaccctaattatatgcaaactacgcaactgaaaatatagtcctaagcaagtttttaaccggcaaacgtcgagtcttcttccggcgatctttcggcagacttctgatatacctttggatttcttctagcggactcctagtagggtcccgatcttgtggcgagtttagcgagtagccgaaccttctcggtgatctccgcaaaccgccgatgatctcttcggcagactttcgaaaacttcgacaagtccccgatttcttctcggttggttccgacagcatctctaacgaaacttcggactccttgaatgtccatcgaacttgactccggtaggcttgctttatattttcaggctatcatagttaatcctacatacttaactcaataatatggattagattaattaacccatcaattgatttcatcatcaaaattcgacattcaacaaacatccgtactcaataacccatcaggctatagttacgtgactatctactgtgatccgtacgtgaagttagcgagtcatgatccaggtcgtgtcacttattggccgaacacgtatcccttatccaaatccagtcttctcaactcttctagcctacccgtctctttttttattacttttgaaagaattcaaatcaaaacagatacaaaataacacggtgagacactgtgacatgctagtctctggaaagcattaattcgcgcatccacagacgtcgtcagcttcatcacccactttttcctacataccatgtcgcatggctttgttgatgacagaccaccacaagcttgcctttggttgtgcctaacagagagagagagagagacagaccgatagcctcctcattcaccatgg

(19) United States
(12) Patent Application Publication
(10) Pub. No.: US 2003/0226175 A1
(43) Pub. Date: Dec. 4, 2003

(54) DNA regulatory elements associated with fruit development

(76) Inventors: Gregory D. May, Ardmore, OK (US); Stephanie K. Clendennen, Portland, OR (US); Hugh S. Mason, Ithaca, NY (US); Miguel A. Gomez Lim, Guanajuato (MX); Charles J. Arntzen, Superstition Mountain, AZ (US)

Correspondence Address: BURNS DOANE SWECKER & MATHIS L L P, POST OFFICE BOX 1404, ALEXANDRIA, VA, 22313-1404, US

(21) Appl. No.: 09/892,635

(22) Filed: Jun. 28, 2001

Related U.S. Application Data
(63) Continuation-in-part of application No. 09/160,351, filed on Sep. 25, 1998, now Pat. No. 6,284,946.
(60) Provisional application No. 60/060,062, filed on Sep. 25, 1997.

(51) Int. Cl. A01H 1/00; C12N 15/82; C07H 21/04; C12N 9/10; C12N 5/04

(52) U.S. Cl. 800/283; 800/287; 536/23.2; 435/419; 435/193; 435/320.1

(57) ABSTRACT

The present invention provides isolated and purified genes which are differentially expressed during banana fruit development, and the protein products of these genes. The present invention further provides DNA regulatory elements which are differentially expressed during banana fruit development, chimeric genes comprising these DNA regulatory elements operably linked to heterologous DNA molecules, and plants transformed with said chimeric genes, providing for controlled expression of said heterologous DNA molecules during the development and ripening of the fruit of said plants, or in response to exogenous ethylene signals in said plants. The present invention also provides a method for expression of a heterologous protein in fruit comprising transforming fruiting plants with one or more chimeric genes according to the present invention, exposing said fruit to an endogenous or exogenous ethylene signal, and harvesting fruit containing said heterologous protein. The method of the present invention may further comprise isolated the proteins produced by said method from the harvested fruit. In a particularly preferred embodiment, the heterologous protein is a therapeutic protein, which may be isolated from the harvested fruit, or consumed directly in the transformed fruit by a patient in need of said therapeutic protein.

This application is a continuation-in-part of U.S. application Ser. No. 09/160,351, filed Sep. 25, 1998, which is a continuation of provisional Application 60/060,062, filed on Sep. 25, 1997. This application claims priority of these aforementioned applications under 35 U.S.C. §§ 119 and 120 and the entire content of both of these priority applications is hereby incorporated by reference.

### BACKGROUND OF THE INVENTION

1. Field of the Invention

The present invention relates to genes which are differentially expressed during banana fruit development, the protein products of these genes, and DNA regulatory elements which are differentially expressed during banana fruit development.

2. Description of the Related Art

Bananas represent a crop of great importance to both the world economy and as a means of supplying subsistence to a large portion of the world's population. The global banana export market is about 10% of the world's production with a $4 billion dollar value. Banana fruit are the fourth most important food in the developing world (May, G D et al. (1995) Biotechnology 13:486-492) with approximately 100 million people acquiring their main energy source from bananas. Bananas, like kiwifruit, papayas, and apples, are climacteric fruit, meaning they ripen in association with an ethylene signal. In the ripening process, starch degradation is associated with a respiratory climacteric in the fruit. Banana fruit ripening is characterized by a number of biochemical and physiological changes including fruit softening, changes in peel color and an increase in respiratory activity (Seymour, G B (1993) in: Seymour G B, et al. (eds) Biochemistry of Fruit Ripening, pp 83-106. Chapman & Hall, London). Although ethylene is produced by the fruit, ripening can also be stimulated by the application of exogenous ethylene. Alternatively, endogenous ethylene production may be stimulated, e.g., by exposing fruit to acetylene.

More specifically, the post-harvest physiology of the banana (Musa acuminata cv. Grand Nain) is characterized by initial harvest, a green storage phase, followed by a burst in ethylene production that signals the beginning of the climacteric period. Associated with this respiratory climacteric is a massive conversion of starch to sugars in the pulp, during which the activities of enzymes involved in starch biosynthesis decrease while those involved in starch breakdown and mobilization increase rapidly (Wu et al. (1989) Acta Phytophysiol. Sin. 15:145-152; Agravante et al. (1990) J. Jpn. Soc. Food Sci. Technol. 37:911-915; Iyare et al. (1992) J. Sci. Food Agric. 58: 173-176; Cordenunsi et al. (1995), J. Agric. Food Chem. 43:347-351; Hill et al. (1995) Planta 196:335-343 and 197:313-323). In addition, the rate of respiration rises sharply (Beaudry et al. (1987) Plant Physiol. 8:277-282; Beaudry et al. (1989) Plant Physiol. 91:1436-1444).

Other changes that occur during ripening include: fruit softening as a result of enzymatic degradation of structural carbohydrates (Agravante et al. (1991) J. Jpn. Soc. Food Sci. Technol. 38:527-532; Kojima et al. (1994) Physiol. Plant. 90:772-778); a decline in those polyphenol compounds responsible for the astringency of the green unripe fruit which are catalyzed by polyphenol oxidase and peroxidases (Mendoza et al. (1994) in I Uritani et al., eds., Postharvest Biochemistry of Plant Food-Materials in the Tropics. Japan Scientific Societies Press, Tokyo, pp 177-191); an increase in the activity of alcohol acetyltransferase, the enzyme that catalyzes the synthesis of isoamyl acetate—the major aroma compound of banana fruit (Harada et al. (1985) Plant Cell Physiol. 26:1067-1074); and a de-greening of the peel as a result of chlorophyll breakdown by chlorophyllase (Thomas et al. (1992) Int. J. Food Sci. Technol. 27:57-63). Stages of banana fruit ripening are scored by peel color index (PCI) numbers, on a scale from 1—very green, to 7—yellow-flecked with brown flecks (Color Preferences Chart, Customer Services Department, Chiquita Brands, Inc.,). PCI can be correlated with other biochemical and physiological parameters associated with fruit development and ripening such as ethylene biosynthesis and respiratory rate. The respiratory rate usually peaks at PCI 2 and PCI 4, respectively, in ethylene-treated bananas (Agravante et al. (1991) supra).

Associated with the respiratory climacteric is a large increase in the rate of protein synthesis (Mugugaiyan (1993) Geobios, 20:18-21), as well as differential protein accumulation (Dominguez-Puigjaner et al. (1992) Plant Physiol. 98:157-162). Poly-galacturonase (PG) has been identified as a protein that increases in banana pulp during ripening, as determined by 2-D gel electrophoresis and immuno-hybridization (id.). Many of the changes that occur during ripening require de novo protein synthesis (Areas et al. (1988) J. Food Biochem. 12:51-60); therefore, a secondary approach to investigate changes that occur during ripening is to isolate transcripts encoding proteins associated with the ripening process. Analogous studies of differential gene expression have been successfully employed in other plant species.

Other enzymes associated with developing and ripening of fruit include proteinase inhibitors and chitinases (Dopico et al. (1993) Plant Molec. Bio. 21:437), stress-related enzymes (Ledger et al. (1994) Plant Molec. Biol. 25:877), β-oxidation pathway enzymes (Bojorquez et al. (1995), Plant Molec. Biol. 28:811), and metabolite-detoxifying enzymes (Picton et al. (1993) Plant Molec. Biol. 23:193). Chitinases are abundant proteins found in a wide variety of plants. Although chitinases are produced by a diversity of plant species, the presence of chitin has not been reported in higher plants. Since chitin is the major structural component of fungal cell walls, it has been proposed that chitinases serve as defense proteins with antifungal activity. Chitinases are reported to be induced in higher plants by a number of different types of stress (Linthorst (1991) Crit. Rev. Plant Sci. 10: 123; Punja et al. (1993) J. Nematol. 25:526; Collinge et al. (1993) Plant J. 3:31). Many plant chitinases are expressed constitutively, although at a low level.

As noted above, in ripening climacteric fruit, starch degradation is associated with a respiratory climacteric in the fruit. Reactive oxygen species (ROS) are byproducts of cellular respiration, especially under conditions which result in high levels of NADH. ROS generation during respiration may be at the site of ubiquinones in the electron transport chain. Both yeast and mammalian metallothioniens may play a direct role in the cellular defense against oxidative stress by functioning as antioxidants (Dalton et al. (1994) Nucl. Acids Res. 22:5016-5203; Tamai et al. (1993) Proc Nat Acad Sci (USA) 90:8013-8017; Bauman et al. (1991) Toxicol. Appl. Pharmacol. 110:347-354). MT may play an additional role in supplying metal ions to Cu- and Zn-superoxide dismutase (SOD), an enzyme that catalyzes the disproportionation of superoxide anion to hydrogen peroxide and dioxygen and is thought to play an important role in protecting cells from oxygen toxicity.

Transcripts encoding MT or MT-like proteins have been isolated from many different plants (recently reviewed in Robinson et al. (1993) Biochem J. 295: 1-10). There is accumulating evidence that the plant MT mRNAs are translated, and the protein may have a function in the plant tissues from which transcripts have been isolated. A seed-associated polypeptide (E_{c} protein) has been purified from wheat and sequenced (Kawashima et al. 1992), and more recently, MT was reported to have been isolated from Arabidopsis (meeting abstract). Based on deduced amino acid sequences, plant MT proteins are approximately 70 aa and have characteristic cysteine-rich regions at the N and C termini, separated by a variable spacer region. Based on the number and distribution of the cysteine residues, plant MTs have been classified into two distinct types (Robinson et al. (1993), supra). Type 1 MTs have 6 N-terminal and 6 C-terminal cysteine residues, whereas type 2 have 8 cysteine residues in the N-terminal domain and 6 at the C-terminus. Although there are no strict patterns of MT expression, in general type 1 transcript abundance is high in roots, and is often metal-inducible, whereas type 2 is expressed primarily in leaves. Other transcripts have been isolated that encode proteins with homology to plant MTs but cannot be classified as either type 1 or type 2, and these include seed-specific proteins or transcripts from barley and wheat (see, Robinson et al. (1993), supra). In Arabidopsis thaliana, MT proteins are encoded by a gene family containing five members, two copies encoding a type 2 MT and 3 encoding a MT with homology to type 1 (Zhou et al. (1995) Mol. Gen. Genet. 248:318-328).

In plants transcripts encoding metallothionein-like proteins have often been isolated by differential screening. Type 2 MT have recently been isolated from plants expressed in association with senescence, leaf abcission (Coupe et al. (1995) Planta 197:442-447), and fruit ripening (Ledger et al. (1994) Plant Molec. Biol. 25:877-886). Using differential screening, Ledger and Gardner (id.) found transcripts encoding MT-like proteins in developing kiwifruit. One, pKIWI503, was specifically upregulated late in fruit development, during ripening of the mature fruit.

A major component of the export market is the level of ripening control which is exerted by modern banana shipping systems. Bananas for export must be shipped under refrigeration at 12-14° C., often under controlled atmosphere (CA) conditions (i.e., low oxygen combined with CO₂), which reduces the effects of ethylene produced by the fruit. Exposure to ethylene for 24 hours at concentrations of 100-1000 μl per liter is used to trigger the ripening climacteric. This “gassing” step is typically done near the final point in the distribution system. Although this system is entirely functional, resulting in marketability of high quality fruit with minimal losses, there remains a role for engineered ethylene control in the banana export market. Bananas for export are harvested green at approximately 75% of full size. This is done to ensure, even with the use of low temperature and CA, that few if any of the bananas start ripening during shipment. Allowing the bananas to remain on the plant longer would result in more carbohydrate accumulation to the fruit and a direct, zero cost increase in yield. If engineered ethylene control were implemented in banana, this increased yield would come at no increased risk of premature ripening during shipment.

Moreover, linking exogenous genes to isolated gene promoters that are differentially expressed during banana ripening, and in response to ethylene, would allow for the production of exogenous protein in banana tied to the ripening process, and in other plants, controlled by ripening or exposure to ethylene.

### SUMMARY OF THE INVENTION

Accordingly, a major object of the present invention is to provide isolated and purified genes which are differentially expressed during banana fruit development, and to provide the protein products of these genes.

A further object of the present invention is to provide DNA regulatory elements which are differentially expressed during banana fruit development, and chimeric genes comprising these DNA regulatory elements operably linked to heterologous DNA molecules, and plants transformed with said chimeric genes, providing for controlled expression of said heterologous DNA molecules during the development of the fruit of said plants, or in response to exogenous development signals, such as ethylene signals in said plants.

A still further object of the present invention is to provide a method for expression of a heterologous protein in fruit comprising transforming fruiting plants with one or more chimeric genes according to the present invention, exposing said fruit to the appropriate natural or exogenous development signal, such as an ethylene signal, and harvesting fruit containing said heterologous protein. The method of the present invention may further comprise isolating the proteins produced by said method from the harvested fruit. In a particularly preferred embodiment, the heterologous protein is a therapeutic protein, which may be isolated from the harvested fruit, or consumed directly in the transformed fruit by a patient in need of said therapeutic protein.

With the foregoing and other objects, advantages and features of the invention that will become hereinafter apparent, the nature of the invention may be more clearly understood by reference to the following detailed description of the preferred embodiments of the invention and to the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Relative abundance of ripening-associated transcripts in banana pulp at PCI 1, 3 and 5. Plasmids containing the indicated cDNA were affixed to nylon membrane and hybridized with pulp radio-labeled first-strand cDNAs. Relative transcript abundance is expressed in arbitrary units (AU).

FIG. 2. Northern analyses of total RNA from pulp and peel (at PCI 3), root, corm, and leaf tissues hybridized with cDNA probes representing each of the eleven classes of differentially expressed transcripts. Putative identities of each transcript are indicated to the left of the panel.

FIG. 3. Total banana pulp protein extract at different stages of ripening, separated by SDS-PAGE and stained with Coomassie blue. Protein profiles during ripening show the presence of an abundant protein of 31 kDa that decreases in relative abundance during ripening.

FIG. 4. Western blot analysis of total soluble protein extracted from different banana tissues and hybridized with polyclonal antiserum against purified P31. The antiserum detects a 31 kDa protein in pulp which is not present in peel, meristem, leaf, corm, or root tissue.

FIG. 5. Expression of P31 (top panel) and pBAN3-30 (bottom panel) in banana pulp during ripening. Total protein and RNA were isolated from banana pulp at each of seven stages of banana fruit ripening (PCI 1 through 7, numbered at top of figure). Pulp proteins were separated by SDS-PAGE and hybridized with the P31 antiserum. Total RNA (10 μg per lane) was separated by agarose gel electrophoresis and transferred to nylon membrane, and hybridized with a ³²P-labeled banana chitinase cDNA probe (pBAN3-30). Both the P31 protein and the corresponding chitinase transcript at 1.2 kilobases are abundant in pulp during the early stages of ripening but decrease as ripening progresses.

FIG. 6. Western blot analysis of the translation products of four banana chitinase cDNA clones homologous to pBAN3-30 expressed as fusion proteins with β-galactosidase in pBluescript and hybridized with P31 antiserum. The polyclonal antiserum recognizes a 35 kDa polypeptide in bacterial cultures containing in-frame cDNA inserts (pBAN3-36 and pBAN3-45) that is not present in bacterial cells containing either the pBluescript cloning vector without an insert (no insert) or chitinase cDNA inserts that are not in-frame with the β-galactosidase gene (pBAN3-30 and pBAN3-31).

FIG. 7. SEQ ID NO: 1-2 Complete nucleotide sequence of the cDNA clone pBAN3-30 and deduced amino acid sequence of the pBAN3-30 translation product. The N-terminal amino acid sequence obtained from purified P31 is aligned with the translation product and underlined, and is identical to the deduced amino acid sequence of pBAN3-30 at 17 of 20 residues. The translation initiation codon ATG starting at position 55 of pBAN3-30 is underlined as well as the in-frame stop codon at position 1024. Other features of the cDNA sequence include several putative polyadenylation signals between positions 1136 and 1148 (underlined).

FIG. 8. SEQ ID NO: 3-8 Amino acid alignments of A) amino- and B)-carboxy-terminal regions of banana P31 with class III acidic chitinase sequences from chickpea (Cicer arietinum, 16), grape (Vitis vinifera, Busam et al. unpublished), Arabidopsis thaliana (17), tobacco (Nicotiana tabacum, 18), sugar beet (Beta vulgaris, 19). Dots indicate the amino acid residues identical to the banana P31 amino acid sequence on the top line. Dashes indicate gaps introduced to aid the alignment. A) Amino-terminal alignment illustrates the lack of sequence homology of the signal-peptide sequence of plant chitinases. B) The carboxy-terminal region indicates the 18 residue C-terminal extension unique to the banana P31 sequence.

FIG. 9. SEQ ID NO: 9-10 cDNA sequences of MT F-1 and F-3.

FIG. 10. A) SEQ ID NO: 11-15 Alignment of deduced amino acid sequences of banana and kiwifruit, apple and papaya fruit-associated metallothionein-like proteins. Alignment was performed using Clustal (default settings). Amino acid alignment of fruit-associated MTs. Asterisks above the sequence indicate the pattern of conserved cysteine residues. A dash denotes a gap inserted in the sequence to aid in alignment. A dot indicates that the amino acid in that position is identical to the banana F1 sequence on the top line. (The total number of amino acids is indicated in parentheses at the end of the sequence.) B) Phylogenetic tree of plant MT sequences indicating that the fruit-associated MT are distinct from MT1 and MT2. GenBank Accession numbers for sequences: banana F1; banana F3; kiwifruit (1-2781 1); papaya (EMBL Y08322); apple (U61974); white spruce (L47746); Vicia faba MT1b (X91078); chickpea MT1 (Cicer arietinum) (X95708); P. sativum MT (Z23097); Oryza sativa MT-2 (D89931); banana MT2; L. esculentum MT-2 (Z68138); Arabidopsis thaliana MT2b (U1 1256); Arabidopsis thaliana MT1b (U1 1254); Arabidopsis thaliana MT1a (U1 1253).

FIG. 11. Northern blot analysis of MT transcript distribution in banana. Total RNA (5 μg/lane) from different banana tissues was separated in a formaldehyde-containing 2% agarose gel, transferred to nylon membrane, and hybridized with an F1 or F3 cDNA probe. The large transcript hybridizes more strongly to the F1 probe, and is approximately 540 bases. The smaller transcript hybridizes more strongly to the F3 cDNA probe, and is approximately 370 bases. Lane labels: Pu=pulp; Pe=perl; R=root; C=corm; L=leaf.

FIG. 12. Restriction maps of MT genomic clones. The maps represent the coding region and at least 1 kb of flanking DNA. The approximate scale is indicated by a dark bar.

FIG. 13. SEQ ID NO: 16 Nucleotide sequence of MT F3 genomic clone, from the 5′ Hindll site to the 3′ Sall site. A 10-base 5′ sequence motif beginning at −313 from the translation start site (in capital letters) shares homology with an antioxidant response element. The putative TATA box (starting at position −96 from the translation start site) is underlined, and the three exons (beginning from the translation start site) are depicted in capital letters. At the 3′ end of the sequence, the stop codon is underlined, as well as a potential polyadenylation signal (TAAATAAA).

FIG. 14. Relative MT transcript abundance in banana pulp-derived protoplasts increases in the presence of hydrogen peroxide but not metal ions, as compared to the untreated control. RNA dot-blots were hybridized to the F3 cDNA probe and hybridization signal intensity, expressed in arbitrary units (AU), was normalized to 18S rRNA as a measure of total RNA loaded.

FIGS. 15A-E. SEQ ID NO: 17-21 Gluc. DNA and amino acid sequence.

FIGS. 16A-I. SEQ ID NO: 22-26 Endo. DNA and amino acid sequence.

FIGS. 17A-G. SEQ ID NO: 27-31 Chitinase DNA and amino acid sequence.

FIGS. 18A-C. SEQ ID NO: 32-36 MT/F1 DNA and amino acid sequence.

FIGS. 19A-C. SEQ ID NO: 37-41 F1/MT#2 DNA and amino acid sequence.

FIG. 20. Structural map of pKS-31G. The banana p31 promoter is located between the BamHI (609) and NcoI sites, and the GUS coding sequence is located between the NcoI and BamHI (4657) sites. The polyadenylation signal present in the CaMV 35S 3′ end is located between the XbaI (4663) and the PstI sites.

FIG. 21. Structural map of pGPT-31G. The expression cassette “p31-GUS-35S” from pKS-31G was placed in the T-DNA vector pGPTV-KAN. The T-DNA right and left borders delineate the DNA that is integrated into the plant nuclear genome during transformation mediated by Agrobacterium.

Selection of transformed plants is facilitated by expression of the NptII gene, which confers resistance to kanamycin, adjacent to the left T-DNA border.

FIG. 22. GUS staining of nontransgenic TA234 and transgenic pGPT-31G tomato fruits. Fruits from fully red-ripe (upper) or pink/red fruit (lower) were stained with X-gluc as described (Jefferson, R. A. (1987), Plant Mol. Biol. Rep. 5:387-405; Jefferson et al. (1987), EMBO J. 13:3901-3907). No staining is seen in control fruits, while staining in transgenic fruits is seen mostly in the vascular and placental tissues.

### DETAILED DESCRIPTION OF THE PREFERRED EMBODIMENTS OF THE INVENTION

The present invention provides isolated and purified banana proteins which are differentially produced in banana fruit during ripening. In a preferred embodiment, said proteins are selected from the group consisting of starch synthases, granule-bound starch synthases, chitinases, endochitinases, β-1,3 glucanases, thaumatin-like proteins, ascorbate peroxidases, metallothioneins, lectins, and other senescence-related genes.

The proteins of the present invention may be isolated from ripening fruit using protein purification methods well known in the art. In particular, fruit containing the protein of the present invention may be subjected to chromatographic techniques which separate proteins present in the extract according to size, affinity and charge. Fractions obtained from each chromatographic step are analyzed for the desired enzymatic activity and subjected to further purification steps. A particularly preferable method for obtaining purified proteins according to the present invention is high performance liquid chromatography (HPLC).

After a protein according to the present invention has been purified, its amino acid sequence can be determined using amino acid sequencing methods well known in the art. A particularly preferable method is Edman degradation. Having obtained sequence information on the protein of the present invention, one can design oligonucleotide probes for isolating the DNA encoding the protein of the present invention, using conventional screening methods, or amplification methods such as polymerase chain reaction (PCR). It is particularly preferable to design such oligonucleotides in a completely degenerate manner, such that oligonucleotides containing each codon encoding a particular amino acid are present in the oligonucleotide mix. Alternatively, inosine can be used at positions in the codon where degeneracies are known to be present. In a particularly preferred embodiment, the proteins of the present invention are encoded by a DNA molecule selected from the group consisting of clones pBAN 3-33, pBAN 3-18, pBAN 3-30, pBAN 3-24, pBAN 1-3, pBAN 3-28, pBAN 3-25, pBAN 3-6, pBAN 3-23, pBAN 3-32, and pBAN 3-46.

The present invention thus further provides an isolated and purified banana DNA molecule which is differentially expressed in fruit during ripening. More specifically, the present invention provides a DNA molecule which is differentially expressed in fruit during ripening, wherein said DNA molecule encodes a protein selected from the group consisting of a starch synthase, a granule-bound starch synthase, a chitinase, an endochitinase, a β-1,3-glucanase, a thaumatin-like protein, an ascorbate peroxidase, a metallothionein, a lectin, or another senescence-related gene. In a particularly preferred embodiment, these DNA molecules are the clones pBAN 3-33, pBAN 3-18, pBAN 3-30, pBAN 3-24, pBAN 1-3, pBAN 3-28, pBAN 3-25, pBAN 3-6, pBAN 3-23, pBAN 3-32, and pBAN 3-46. In another preferred embodiment, the DNA molecule of the present invention has a nucleotide sequence selected from the group consisting of SEQ ID NO: 1; SEQ ID NO: 2; and SEQ ID NO: 3.

In general, the procedures for isolating the DNA encoding a protein according to the present invention, subjecting it to partial digestion, isolating DNA fragments, ligating the fragments into a cloning vector, and transforming a host are well known in recombinant DNA technology. Accordingly, one of ordinary skill in the art can use or adapt the detailed protocols for such procedures as found in Sambrook et al. (1989), Molecular Cloning: A Laboratory Manual, 2nd. Ed., Cold Spring Harbor Laboratory, Cold Spring Harbor, N.Y., 3 volumes, or in any other manual on recombinant DNA technology.

Once the gene encoding a protein of the present invention has been obtained from one species, it can serve as a hybridization probe to isolate corresponding genes from the other species by cross-hybridization under low to moderate stringency conditions. Such conditions are usually found empirically by determining the conditions wherein the probe specifically cross-hybridizes to its counterpart gene with a minimum of background hybridization. Nucleic acid hybridization is a well known technique and thoroughly detailed in Sambrook et al.

As noted above, the DNA encoding the proteins of the present invention can be originally isolated using PCR. Corresponding DNAs from other species can also be isolated using PCR, and oligonucleotides for performing these subsequent PCR reactions can be optimized using the sequence information obtained from DNA cloned from the first species.

Moreover, peptides and fragments as well as chemically modified derivatives of the proteins of the present invention are also contemplated by the present invention. Briefly, any peptide fragment, derivative or analog which retains substantially the same biological activity of the protein of the present invention, and is differentially produced during fruit ripening, is contemplated. An analog may be defined herein as a peptide or fragment which exhibits the biological activity of the protein of the present invention, and which is differentially expressed during fruit ripening, but which has an amino acid substitution, insertion or deletion in comparison to the wild-type protein. Such an analog can be prepared by the “conservative” substitution of an amino acid having similar chemical properties. One of ordinary skill in the art can readily identify suitable substitions.

Thus, it should also be appreciated that also within the scope of the present invention are DNA sequences encoding a protein according to the present invention having the same amino acid sequence as the wild-type protein, but also those DNA sequences which are degenerate to the wild-type sequence. By “degenerate to” is meant that a different three-letter codon is used to specify a particular amino acid. It is well known in the art that the following codons can be used interchangeably to code for each specific amino acid:

| Amino | | |
|---|---|---|
| Acid | Abbrev. | Codons |
| Phenyla- | (Phe or | UUU, UUC |
| lanine | F) | |
| Leucine | (Leu or | UUA, UUG, CUU, CUC, |
| | L) | CUA, CUG |
| Isoleu- | (Ile or | AUU, AUC, AUA |
| cine | I) | |
| Methio- | (Met or | AUG |
| nine | M) | |
| Valine | (Val or | GUU, GUC, GUA, GUG |
| | V) | |
| Serine | (Ser or | UCU, UCC, UCA, UCG, |
| | S) | AGU, AGC |
| Proline | (Pro or | CCU, CCC, CCA, CCG |
| | P) | |
| Threonine | (Thr or | ACU, ACC, ACA, ACG |
| | T) | |
| Alanine | (Ala or | GCU, GCG, GCA, GCG |
| | A) | |
| Tyrosine | (Tyr or | UAU, UAC |
| | Y) | |
| Histidine | (His or | CAU, CAC |
| | H) | |
| Glutamine | (Gln or | CAA, CAG |
| | Q) | |
| Aspara- | (Asn or | AAU, AAC |
| gine | N) | |
| Lysine | (Lys or | AAA, AAG |
| | K) | |
| Aspartic | (Asp or | GAU or GAC |
| Acid | D) | |
| Glutamic | (Glu or | GAA or GAG |
| Acid | E) | |
| Cysteine | (Cys or | UGU or UGC |
| | C) | |
| Arginine | (Arg or | CGU, CGC, CGA, CGG, |
| | R) | AGA, AGG |
| Glycine | (Gly or | GGU, GGC, GGA, GGG |
| | G) | |
| Stop | | UAA (ochre), UAG |
| codon | (amber), UGA (opal) | |

It should be understood that the codons specified above are for RNA sequences. The corresponding codons for DNA have T substituted for U.

Mutations can be made in the wild-type sequence such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The following is one example of various groupings of amino acids:

Amino acids with nonpolar R groups

| | |
|---|---|
| Alanine | Proline |
| Valine | Phenylalanine |
| Leucine | Tryptophan |
| Isoleucine | Methionine |

Amino acids with uncharged polar R groups

| | |
|---|---|
| Glycine | Tyrosine |
| Serine | Asparagine |
| Threonine | Glutamine |
| Cysteine | |

Amino acids with charged polar R groups
(negatively charged at Ph 6.0)

| | |
|---|---|
| Aspartic acid | Glutamic acid |

Basic amino acids (positively charged at pH 6.0)

| | |
|---|---|
| Lysine | Arginine |
| Histidine (at pH 6.0) | |

Another grouping may be according to molecular weight (i.e., size of R groups):

| | | | |
|---|---|---|---|
| Glycine | 75 | Aspartic acid | 133 |
| Alanine | 89 | Glutamine | 146 |
| Serine | 105 | Lysine | 146 |
| Proline | 115 | Glutamic acid | 147 |
| Valine | 117 | Methionine | 149 |
| Threonine | 119 | Histidine (at pH 6.0) | 155 |
| Cysteine | 121 | Phenylalanine | 165 |
| Leucine | 131 | Arginine | 174 |
| Isoleucine | 131 | Tyrosine | 181 |
| Asparagine | 132 | Tryptophan | 204 |

Another grouping may be those amino acids with phenyl groups:

| | |
|---|---|
| Phenylalanine | Tryptophan |
| Tyrosine | |

Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

Amino acid substitutions may also be introduced to substitute an amino acid with a particularly preferable property. For example, a Cys may be introduced at a potential site for disulfide bridging with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

Purification of the proteins of the present invention from natural or recombinant sources can be accomplished by conventional purification means such as ammonium sulfate precipitation, gel filtration chromatography, ion exchange chromatography, adsorption chromatography, affinity chromatography, chromatofocusing, HPLC, FPLC, and the like. Where appropriate, purification steps can be done in batch or in columns.

Peptide fragments of the proteins of the present invention can be prepared by proteolysis or by chemical degradation. Typical proteolytic enzymes are trypsin, chymotrypsin, V8 protease, subtilisin and the like; the enzymes are commercially available, and protocols for performing proteolytic digests are well known. Peptide fragments are purified by conventional means, as described above. Peptide fragments can often be identified by amino acid composition or sequence. Peptide fragments are useful as immunogens to obtain antibodies against the proteins of the present invention.

In accordance with the present invention, all or a part of a DNA molecule according to the present invention can be stably inserted in a conventional manner into the nuclear genome of a plant cell, and the so-transformed plant cell can be used to produce a transgenic plant showing improved expression of the DNA molecule according to the present invention. In this regard, a disarmed Ti-plasmid, containing a DNA molecule according to the present invention, in Agrobacterium (e.g., A. tumefaciens) can be used to transform a plant cell using the procedures described, for example, in EP 116.718 and EP 270,822, PCT publication 84.02913, EPA 87400544.0 and Gould et al. ((1991) Plant Physiol. 95: 426) which are incorporated herein by reference). Preferred Ti-plasmid vectors contain the foregoing DNA sequence between the border sequence, or at least located to the left of the right border sequence, of the T-DNA of the Ti-plasmid.

Replicable expression vectors according to the present invention may include a promoter, a transcription enhancer element, a termination signal, a translation signal, or a combination of two or more of these elements, generally including at least a promoter element.

Replicable expression vectors are generally DNA molecules engineered for controlled expression of a desired gene, especially where it is desirable to produce large quantities of a particular gene product, or polypeptide. The vectors comprise one or more nucleotide sequences operably linked to a gene to control expression of that gene, the gene being expressed, and an origin of replication which is operable in the contemplated host. Preferably the vector encodes a selectable marker, for example, antibiotic resistance. Replicable expression vectors can be plasmids, bacteriophages, cosmids and viruses. Any expression vector comprising RNA is also contemplated. The replicable expression vectors of this invention can express the protein of the present invention at high levels. Many of these vectors are based on pBR322, M13 and lambda and are well known in the art and employ such promoters as tip, lac, P_{L}, T7 polymerase and the like. Hence, one skilled in the art has available many choices of replicable expression vectors, compatible hosts, and well-known methods for making and using the vectors.

Other types of vectors can be used to transform plant cells, using procedures such as direct gene transfer (as described, for example, in EP 233,247), pollen mediated transformation (as described, for example, in EP 270,356, PCT publication WO 95/01856, and U.S. Pat. No. 4,407,956), liposome-mediated transformation (as described, for example, in U.S. Pat. No. 4,5376,475) and other methods such as the methods for transforming monocots described in Fromm et al. ((1990) Bio/Technology 8:833) and Gordon-Kamm et al.((1990) Plant Cell 2:603).

Preferably, the gene according to the present invention is inserted in a plant genome downstream of, and under the control of, a promoter which can direct the expression of the gene in the plant cells. Preferred promoters include, but are not limited to, the strong constitutive 35S promoter (Odell et al. (1985) Nature 313:810) of cauliflower mosaic virus; 35S promoter have been obtained from different isolates (Hull et al. (1987) Virology 86:482). Other preferred promoters include the TR1′ promoter and the TR2′ promoter (Velten et al.(1984) EMBO J. 3:2723) Alternatively, a promoter can be utilized which is not constitutive but rather is specific for one or more tissues or organs. For example, a gene according to the present invention can be selectively expressed in the green tissues of a plant by placing the gene under control of a light-inducible promoter such as the promoter of the ribulose-1,5-phosphate-carboxylase small subunit gene as described in EPA 8300921.1. Another alternative is to use a promoter whose expression is inducible by temperature or chemical factors.

It as also preferred that a gene according to the present invention be inserted upstream of suitable 3′ transcription regulation signals (i.e., transcript 3′ end formation and polyadenylation signals) such as the 3′ untranslated end of the octopine synthase gene (Gielen et al.(1984) EMBO J., 3:835-845) or T-DNA gene 7 (Velten and Schell (1985) Nucl. Acids Res. 13:6981-6998).

The resulting transformed plant of this invention expresses the inserted gene and is characterized by the production of high levels of the gene product. Such a plant can be used in a conventional breeding scheme to produce more transformed plants with the same improved phenotypic characteristics, or to introduce the gene into other varieties of the same or related plant species. Seeds, which are obtained from transformed plants, contain the gene as a stable genomic insert.

The present invention further encompasses compositions comprising one or more proteins according to the present invention, and a carrier therefor.

The present invention also provides isolated and purified banana DNA regulatory elements which are 5′ or 3′ to a gene which is differentially expressed during fruit development. In a preferred embodiment, said DNA regulatory elements are promoters. In a particularly preferred embodiment, said promoter is the 2.15 kb 5′ upstream region of the p31 gene whose nucleotide sequence is given in SEQ ID NO: 44. In another particularly preferred embodiment, said p31 promoter is modified with NcoI enzyme sites for vector insertion and whose nucleotide sequence is given in SEQ ID NO: 45. It will be appreciated by those skilled in the art that modifications can be made to the promoters without destroying the scope or spirit of the invention. The invention contemplates that nucleotide fragments of the promoters may be altered, added, or deleted without substantially affecting the promoter's ability to drive gene expression. Verification that a particular modification does not adversely impact promoter activity is easily determined by common reporter gene assays such as the one illustrated in Example 4. Said regulatory elements of the present invention control the expression of genes to which they are operatively linked, and are sensitive to a plant development signal. In a preferred embodiment, the plant development signal is an ethylene signal. The ethylene signal may be ethylene gas released by ripening fruit, either naturally or stimulated artificially; alternatively, the ethylene signal is produced by exposure of the plant or fruit to exogenous ethylene gas.

The DNA regulatory elements of the present invention may be linked to native plant genes via homologous recombination, e.g., via the method of U.S. Pat. No. 5,272,071, the contents of which are incorporated herein by reference. Alternatively, the DNA regulatory elements of the present invention may be operatively linked to a DNA molecule which is desired to be expressed in a plant in response to a development signal, thus forming a chimeric gene. Transformation of plants with such a chimeric gene, as described above, provides for controlled expression in fruit encoded by said DNA molecule. In a particularly preferred embodiment, said DNA molecule encodes a therapeutic protein.

The DNA molecules of the present invention may be used to transform any plant in which expression of the particular protein encoded by said DNA molecules is desired. In addition, the regulatory elements of the present invention may be used to trigger gene expression in any plant in which gene expression is desired. Suitable plants for transformation with the DNA molecules and regulatory elements of the present invention include Banana (e.g., Musa acuminata); kiwifruit (e.g., Actinidia deliciosa); grape (e.g., Vitis vinifera, V. labrusca, V. rotundifolia); peach, nectarine, plum, apricot, cherry, almond (e.g., Prunus persica, P. domestica, P. salicina, P. avium, P. cerasus, P. amygdalus); pear (e.g., Pyrus communis, P. pyrifolia.); apple (e.g., Malus x domestica); eggplant (e.g., Solanum melongena); tomato (e.g., Lycopersicon lycopersicum, L. esculentum); peppers (e.g., Capciscum sp.); peas and beans (e.g., Phaseolus vulgaris, P. lunatus, P. Limensis, Cicer arietimum, Vigna angularis, Pisum sativum, Glycine max); cucumbers, melons, squash and pumpkins (e.g., Cucumis melo, C. sativus, Citrullus lanatus, Cucurbita maxima, C. pepo); maize (e.g., Zea mays); rice (e.g., Oryza sativa); wheat; barley (e.g., Hordeum vulgare); tobacco (e.g., Nicotiana tabacum); potato (e.g., Solanum tuberosum); beet (e.g., Beta vulgaris); carrot (e.g., Daucus carota); parsnip (e.g., Pastinaca sativa); turnip, rutabaga (e.g., Brassica rapa, B. napus); and radish (e.g., Raphanus sativus). It will be understood that this is not an exclusive list, but merely suggestive of the wide range of utility of the DNA molecules and regulatory elements of the present invention.

The present invention thus also provides a method for expression of heterologous protein in fruit comprising transforming fruiting plants with a chimeric gene, replicable expression vector, or plasmid comprising a ripening-associated promoter, as described above, exposing said fruit to an ethylene signal, and harvesting fruit containing said heterologous protein. The protein may be isolated from the harvested fruit using conventional methods, including those described above. Alternatively, where the protein is a therapeutic protein, in a preferred embodiment the fruit may be directly consumed by a patient in need of the therapeutic protein, thus providing for convenient oral administration of the protein.

The following examples are presented in order to more fully illustrate the preferred embodiments of the invention. They should in no way be construed, however, as limiting the broad scope of the invention.

### EXAMPLE 1

### Differential Gene Expression in Ripening Banana (Musa acuminata cv. Grand Nain) Fruit

The experiments described in this example were designed to isolate those banana genes that are differentially expressed in ripening banana fruit.

### Materials and Methods

Plant Materials

Ethylene treated and untreated banana fruit (Musa acuminata cv. Grand Nain) were obtained from the Northside Banana Company (Houston, Tex.). The pulp and peel of fruit representing each of the seven different stages of ripening (PCI 1 through 7) were separated and quick-frozen in liquid nitrogen. Tissues from ten individual fruit were pooled to obtain a uniform representative sample for each ripening stage and ground to a fine powder under liquid nitrogen in a stainless steel Waring blender. Ground samples were stored at −80° C. until utilized. Leaf, corm and root tissue were obtained from greenhouse-grown plants (cv Grand Nain), ground in liquid nitrogen using a mortar and pestle, and stored at −80° C.

RNA Isolation

Pre-warmed (65° C.) RNA extraction buffer (1.4% (w/v) SDS, 2% (w/v) polyvinylpyrrolidone, 0.5 M NaCl, 0. 1M sodium acetate, 0.05 M EDTA, pH 8.0, 0.1% (v/v) P-mercaptoethanol) was added to previously ground samples of pulp from PCI 1 and PCI 3 at a 5:1 tissue to buffer ratio. Samples were homogenized with two or three 30 second pulses of a Polytron tissue homogenizer (Brinkman) and incubated at 65° C. for 15 min. Starch and other cell debris were pelleted by centrifugation at 2,400 g for 10 min at room temperature and the supernatant transferred to a disposable 50 ml polypropylene screw-cap tube. After the addition of 0.2 vol. of 5 M potassium acetate, pH 4.8, samples were mixed by inversion and incubated on ice for 30 min. The resulting precipitate was pelleted by centrifugation at 20.2 k rpm for 10 min at 4° C. in a Sorvall SW28 rotor. The supernatant was transferred to a disposable polypropylene centrifuge tube, and the high-molecular weight RNA was precipitated by the addition of lithium chloride to a final concentration of 2.5 M and incubation overnight at 4° C. RNA was isolated from leaf and root tissues using a CTAB isolation buffer modified from Doyle and Doyle (1987). Root and leaf tissues were ground to a powder in liquid nitrogen using a mortar and pestle. Five grams of frozen powder were added to 10 ml of prewarmed (65° C.) CTAB RNA extraction buffer (100 mM Tris-Borate, pH 8.2, 1.4 M NaCl, 20 mM EDTA, 2% (w/v) CTAB (hexadecyltrimethyl-ammonium bromide), 0.1% (v/v) β-mercaptoethanol). Samples were homogenized with two or three 30 second pulses of a Polytron tissue homogenizer (Brinkman), and the homogenate was incubated at 65° C. for one hour. Samples were cooled to room temperature, extracted twice with an equal volume of chloroform, and the phases were separated by centrifugation. Following centrifugation, lithium chloride was added to a final concentration of 2M, and RNA was allowed to precipitate overnight at 4° C. RNA was pelleted at 4° C. for 20 min at 20 kg, washed with 70% ethanol and re-suspended in DEPC-treated H₂O. The RNA was phenol:chloroform (1:1) extracted and ethanol precipitated.

cDNA Library Construction

Pulp PCI 1 and 3 cDNA libraries were generated using poly(A)+ mRNA prepared from total RNA using a magnetic bead separation protocol (Dynal) according to the manufacturer's instructions. Lambda Zap cDNA libraries were generated according to the supplier's protocol (Stratagene).

Differential Screening

Approximately 5×10⁴ plaque-forming units (pfu) from each cDNA library were plated onto LB plates using the appropriate E. coli host strain. Duplicate plaque-lifts were generated by placing Nytran nylon filters (Schleicher and Schuell) onto plaque-containing plates for one and four minutes for the first and second filters, respectively. Filter-bound DNA was denatured for two min in 1.5 M NaCl, 0.5 M NaOH, and neutralized for four minutes in 1.5 M NaCl, 0.5 M Tris (pH 8.0). Filters were rinsed in 0.5 M Tris (pH 8.0), blotted dry, and UV crosslinked (Stratalinker, Stratagene).

Labeled first-strand cDNA probes used in the differential screening were synthesized from 15 mg total RNA in the presence of 1.5 μm [α-[³²P] dCTP (3000 mCi/mmol) using an oligo(dT)₁₅, primer (Promega) and 15U MMLV reverse transcriptase according to the manufacturer's instructions (Promega). The mRNA template was removed by hydrolysis in 100 mm NaOH at 65° C. for 30 min. The reaction was neutralized in 100 mm Tris-HCl (pH 8.0), and the labeled first-strand cDNA was ethanol precipitated in the presence of 20 μg of carrier yeast tRNA.

Filters were pre-hybridized for 30 min in 1 mM EDTA, 0.25 M phosphate buffer (pH 7.2), 7% (w/v) SDS, and hybridized overnight at 65° C. in the same solution containing the denatured probe (1×10⁷ cpm/ml). Hybridized filters were washed twice for 30 min each at 65° C. in Wash Solution One (1 mM EDTA, 40 mM phosphate buffer, pH 7.2, 5 % (w/v) SDS) and three times for 30 min each at 65° C. in Wash Solution Two (1 mM EDTA, 40 mM phosphate buffer pH 7.2, 1 % (w/v) SDS). The air-dried filters were subjected to autoradiography (X-Omat X-ray film, Kodak) for 72 h at −80° C. with an intensifying screen.

Banana pulp cDNA libraries from PCI 1 and PCI 3 were each probed separately and differentially with labeled cDNA from pulp at PCI 1 and PCI 3. Plaques which demonstrated strong differential signal intensities between both probes were selected as positives. Positive plaques were then subjected to secondary screening to purify single isolates by utilizing the same probes as in the primary screening. pBluescript phagemids were excised from the isolated plaques according to the manufacturer's recommendations (Stratagene).

Sequence Analysis

Small-scale alkaline lysis plasmid preparations followed by phenol:chloroform extraction and ethanol precipitation (Sambrook et al., 1989) yielded template plasmid DNA suitable for automated sequencing. Plasmid DNA templates were sequenced, using the T3 primer, on an ABI 373A DNA sequencer (Applied Biosystems, Foster City, Calif.). Vector and 3′ poly(A) residue sequences were removed from the output sequence. Edited sequences were loaded into the NCBI form for BLAST (9. 1) searching on a network server (www.ncbi.nlm.nih.gov), and searches were performed using the default settings of BLASTN (Altschul et al., 1990). For some cDNA clones, no significant homology (defined as a high score above 100) with sequences in the databases was identified using BLASTN. In that event, the default settings of the BLASTX search, an algorithm that translates the nucleic acid sequence in all six frames and searches a non-redundant amino acid database for matches, were used (Gish and States, 1993).

Dot-blot Hybridization

Comparisons of the relative transcript abundance of the individual cDNA clones between PCI 1, 3 and 5 pulp were made through dot-blot hybridization experiments. Plasmids containing the cDNA inserts were affixed to nylon membrane and hybridized with first-strand cDNA from generated from PCI 1, 3 or 5 pulp RNA. The equivalent of 1×10¹¹ copies of each plasmid (approximately 0.5 μg of plasmid DNA containing a 1 kb cDNA insert) was heat denatured (95° C. for 10 min), and quenched on ice. Using a vacuum dot-blot apparatus (BioRad), target DNA was applied to HyBond N+ nylon membrane (Amersham). Membranes were air-dried, UV crosslinked (Stratalinker), and hybridized as described above using 2×10⁶ cpm/ml of PCI 1, 3, and 5 radiolabeled first strand cDNA as probe. Following hybridization, membranes were exposed to a phosphorescent screen (PhosphorImager, Molecular Dynamics) and the scanned image was analyzed with the ImageQuant quantitation software.

Northern Analyses

Total RNA was isolated from banana pulp and peel at PCI 3, and from root, corm, and leaf tissues of greenhouse-grown Grand Nain banana plants. Ten micrograms of each of the RNA samples were separated by electrophoresis through formaldehyde-containing agarose gels and transferred to Nytran Plus nylon membrane (Schleicher and Schuell) using a vacuum transfer apparatus (BioRad) according to the manufacturer's recommendations. Equal RNA loading was confirmed by staining the RNA-containing nylon membranes with methylene blue (Sambrook et al. ,1989). The RNA blots were hybridized with a cDNA probe representing the largest isolate from each of the eleven nonredundant groups of clones. DNA probes were synthesized using the Rad-Prime DNA Labeling System (Gibco BRL), and hybridized as described above.

### Results

Differential screening of approximately 10⁵ plaques with labeled pulp cDNAs resulted in the identification of approximately 100 plaques with a signal intensity sufficient to be detected by autoradiography after a 72 hour exposure to X-ray film. It was apparent from the signal intensities observed between differentially hybridized plaque lifts that the relative abundance of a number of transcripts changed between PCI 1 and 3. A total of 38 cDNA clones were isolated from banana pulp libraries by differential screening.

Sequence alignment and homology searches indicate that eleven non-redundant groups of cDNAs were identified (Table 1). Using sequence homology, BLAST searches were able to assign, with high scores between 167 and 1294, a putative identity for all clones. Amino acid sequence homology searches using the BLASTX algorithm were necessary to assign an identity to the clones encoding the putative lectin and senescence-related protein. According to the results of the sequence homology searches, all of the banana sequences are more similar to other plant genes than to genes from other organisms. There were many redundant isolates, especially of the smaller cDNAs such as those encoding the different metallothioneins. Ten clones encoding a putative chitinase, an especially abundant protein in banana pulp (R. López-Gómez, unpublished data), were isolated.

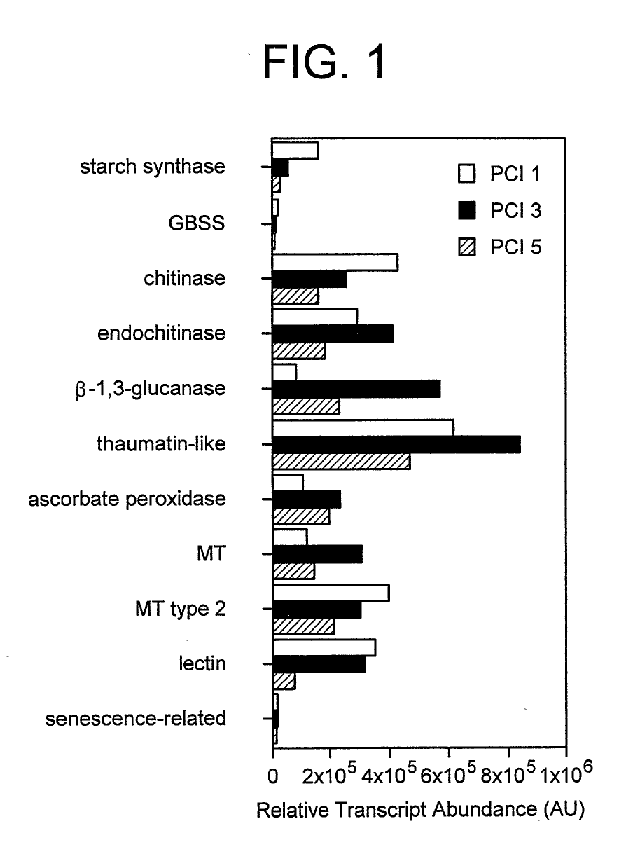

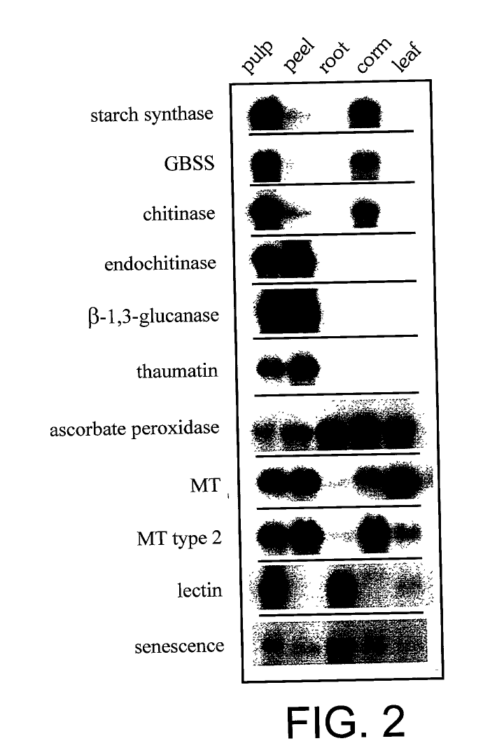

Relative abundance among the different transcripts was estimated by hybridizing isotopically labeled first-strand cDNA to an excess of cloned cDNA which was previously dot-blotted onto nylon membrane. This technique also allowed for the confirmation of differential expression of these transcripts in pulp between PCI 1 and 3, and at a later stage of ripening, PCI 5 (FIG. 1). Relative transcript abundance of starch synthase, GBSS, chitinase, and a type 2 methallothionein decreased in pulp between PCI 1 and 3, and continued to decrease through PCI 5. There was a peak in the abundance of several of the transcripts in PCI 3 pulp, including endochitinase, glucanase, thaumatin, ascorbate peroxidase, and metallothionein. The differential expression of these banana transcripts before and after the peak in ethylene biosynthesis indicates that the transcripts that increase in abundance between PCI 1 and PCI 3 respond to ethylene. The differential expression of the eleven different groups of cDNAs in banana pulp between ripening stages PCI 1 and 3 was confirmed by Northern analyses (data not shown). Results from the dotblot hybridization were also used to estimate relative abundance of each class of cDNA in the pulp of ripening banana fruit, with thaumatin and P-1,3-glucanase being the first and second most abundant transcripts, respectively (FIG. 1).

TABLE I
Genes that are differentially expressed during banana
fruit ripening. Putative cDNA identities are based on
sequence homology. Number of homologous clones isolated
indicated in parentheses. High scores obtained using
BLASTN or BLASTX. Changes in pulp relative transcript
abundance from PCI 1-3 indicated as “up” or
“down” based on dot-blot hybridizations.
Transcript sizes estimated from Northern analyses of
pulp total RNA.

| | | | PCI | Transcript |
|---|---|---|---|---|
| Homology to: | Clone | High Score [P(N)^{a}] | 1 to 3 | size (kb) |
| sweet potato starch synthase (2) | pBAN 3−33 | 198 [6.8e−6] | down | 2.2 |
| cassava GBSS (4) | pBAN 3-18 | 1,121 [6.5e−95] | down | 2.2 |
| winged bean chitinase (10) | pBAN 3-30 | 300 [7.9e−31] | down | 1.2 |
| rice endochitinase (2) | pBAN 3-24 | 773 [3.4e−93 | up | 1.2 |
| soybean β-1,3-glucanase (2) | pBAN 1-3 | 524 [3.4e−33] | up | 1.3 |
| katemfe fruit thaumatin (2) | pBAN 3-28 | 635 [3.0e−125] | up | 1.0 |
| rice ascorbate peroxidase | pBAN 3-25 | 1,294 [4.0e−110] | up | 1. 1 |
| kiwifruit metallothionein (5) | pBAN 3-6 | 218 [1.7e−11] | up | 0.5 |
| castor bean MT type 2^{b} (6) | pBAN 3-23 | 518 [2.4e−33] | down | 0.6 |
| jack fruit lection (α subunit)^{c} (3) | pBAN 3-32 | 177 [2.0e−19] | down | 0.8 |
| asparagus senescence-related gene^{c} | pBAN 3-46 | 167 [3.1e−16] | up | 1.0 |

^{a}Probability of homology occurring by chance (see Altschul et al., 1990)

Although these cDNAs are relatively abundant in the pulp of banana fruit, their patterns of expression are not limited to these tissues. Northern analyses indicate that starch synthase, GBSS, and chitinase transcripts were abundant in pulp and corm tissues, and present in peel. Expression of the endochitinase, thaumatin-like protein, and β-1,3 glucanase transcripts was limited to the pulp and peel of the fruit. Both classes of metallothionein transcripts were expressed in all tissues analyzed, but were most abundant in the pulp and peel. In comparison, MT was more abundant in leaves than Type-2 MT, while the converse was observed in the corm. Lectin transcripts were most abundant in pulp and root tissues, whereas the ascorbate peroxidase and senescence-related protein transcripts were ubiquitously expressed.

Many of the physiological changes that occur during banana fruit ripening are in response to ethylene produced in the pulp (Don-Tinguez and Vendrell, 1993; Burdon et al., 1994). In addition, ethylene also serves as a signal for other physiological changes including senescence. The cDNA clones identified in this study were isolated by differential screening at stages of fruit ripening corresponding to periods before and after the peak in ethylene biosynthesis (Agravante et al., 1991). Therefore, it is likely that some of the transcripts that increase in abundance between those stages of ripening may be regulated by ethylene, even if they do not have a direct role in the ripening process. The role of ethylene in the regulation of PR proteins (glucanase, chitinase, endochitinase, thaumatin) has been well documented. Ethylene is also believed to influence expression of ascorbate peroxidase (Mehlhorn, 1990) and metallothionein (Coupe et al., 1995)

### EXAMPLE 2

### The Abundant 31-Kilodalton Banana Pulp Protein is Homologous to Class-III Acidic Chitinases

The experiments described in this example were designed to identify and characterize the abundant 31 kD protein from the pulp of banana fruit (Musa acuminata cv. Grand Nain), and to isolate a cDNA encoding this protein.

### Materials and Methods

Plant Materials

Ethylene treated and untreated banana fruit (Musa acuminata cv. Grand Nain) were obtained from the Northside Banana Company (Houston, Tex.). The pulp and peel of fruit representing each of the seven different stages of ripening (PCI 1 through 7) were separated and quick-frozen in liquid nitrogen. Tissues from ten individual fruit were pooled to obtain a uniform representative sample for each ripening stage and ground to a fine powder under liquid nitrogen in a stainless steel Waring blender. Ground samples were stored at −80° C. until utilized. Other banana tissues were obtained from greenhouse-grown plants (cv Grand Nain).

Protein Isolation for Antiserum Production, N-terminal Sequencing, and Western Blotting

Soluble banana pulp proteins were differentially precipitated from pulp extracts with ammonium sulfate. P31 was most abundant in the 40 to 60% ammonium sulfate fraction, as determined by SDS-PAGE separation (Laemmli, U.K. (1970) Nature 227:680), followed by Coommassie blue staining (Sambrook et al. (1989) Molecular Cloning, a Laboratory Manual, Ed. 2 Cold Spring Harbor Press, Plainview, N.Y.). The 31 kDa protein band was excised from the gel, homogenized and used to immunize a rabbit for antiserum production, according to standard protocols. In addition, proteins from the 40 to 60% ammonium sulfate fraction were separated by SDS-PAGE and transferred PVDF protein sequencing membrane and stained with Coomassie blue. The stained 31 kDa protein band was excised from the membrane and the N-terminal sequence was determined.

Total protein isolated from banana root, corm, leaf, meristem, peel, and pulp at different stages of ripening were separated by SDS-PAGE and electrophoretically transferred to PVDF membrane. The membranes were incubated with the primary antiserum at 1:500 dilution, and the bound antibodies were visualized using chemiluminescence.

Northern Blot Analyses

Total RNA was isolated from banana leaf, corm, root, peel, and floral structures and from banana pulp at PCI 1 through 7 (López-Gómez, R., et al. (1992) 5:440). Agarose gel electrophoresis, and northern blotting and hybridization were performed according to standard protocols (Sambrook et al., supra). The cDNA clone pBAN3-30 was radiolabeled with ³²P-dCTP by random priming and used as a probe.

pBAN3-30 Isolation and Sequence Analysis

pBAN3-30 was isolated from a banana pulp cDNA library by differential screening (Clendennen, S. K. et al. (1997) Plant Physiology). The complete sequence of the cDNA insert was determined on both strands, and the open reading frame was translated. Sequence homology of pBAN3-30 and the translation product (P31) were determined using the BLAST search algorithm for searching GenBank (Altschul, S. F., et al. (1990) J. Molec. Biol. 215:403). For the amino acid alignments, plant chitinase sequences showing significant homology to P31 were downloaded from GenBank and aligned manually.

Expression of Recombinant P31

A total of ten homologous chitinase clones were isolated from the banana pulp cDNA library by differential screening, including pBAN3-30, pBAN3-31, pBAN3-36, and pBAN3-45 (Clendennen et al., supra). These four clones were used for the expression of P31 for western blot analysis of the translation products. It was determined that pBAN3-36 and pBAN3-45 contained chitinase coding sequences that were in-frame with respect to β-galactosidase in the pBluescript cloning vector. All four of the cDNA clones, in E. coli XL1-blue host cells, were grown to log phase in selective media and then induced by IPTG. Total bacterial proteins were separated by SDS-PAGE and transferred to PVDF membrane. The western blot was hybridized with P31 antiserum and visualized using chemiluminescence.

### Results

P31 Isolation and Tissue Distribution

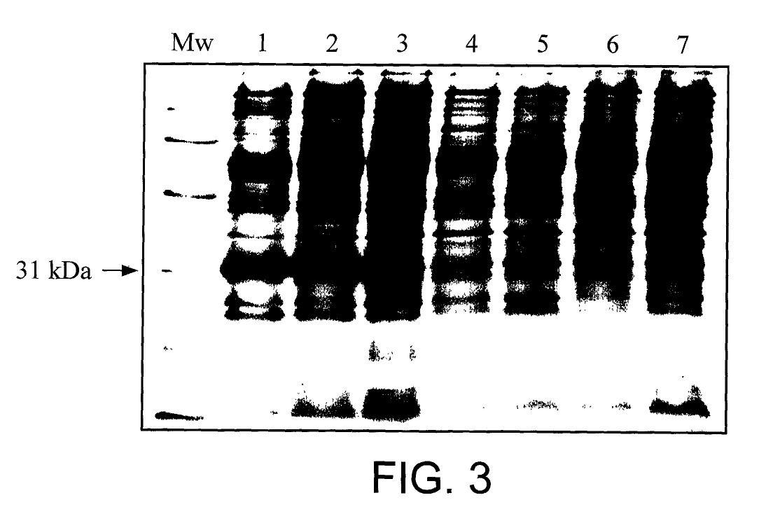

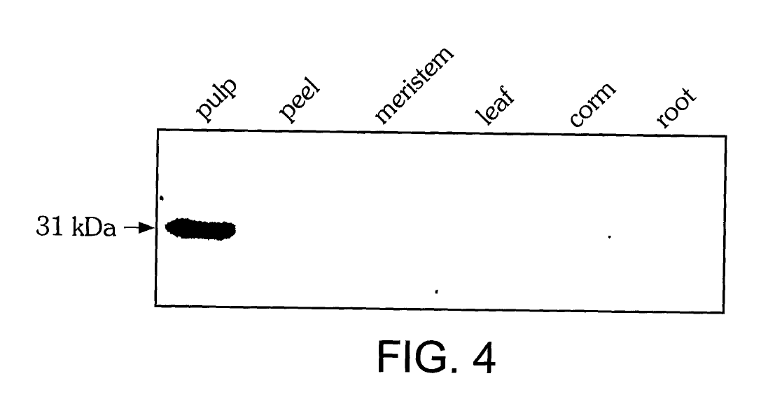

SDS-PAGE analysis of total proteins isolated from pulp of banana fruit at seven ripening stages indicated changes in abundance of several proteins (FIG. 1). The most abundant protein during the pre-climacteric stage (Peel Color Index or PCI 1 and 2) is a 31 kDa protein (P31) which seemed to decrease slightly in abundance as ripening proceeded (FIG. 3). This protein (P31) was partially purified by a combination of ammonium sulfate precipitation and separation by SDS-PAGE. Polyclonal antiserum was raised against the purified protein. The P31 antiserum recognizes a single 31 kDa polypeptide in banana pulp that is not present in banana peel, meristem, corm, or root tissue (FIG. 4). These results indicate that P31 is fruit-specific.

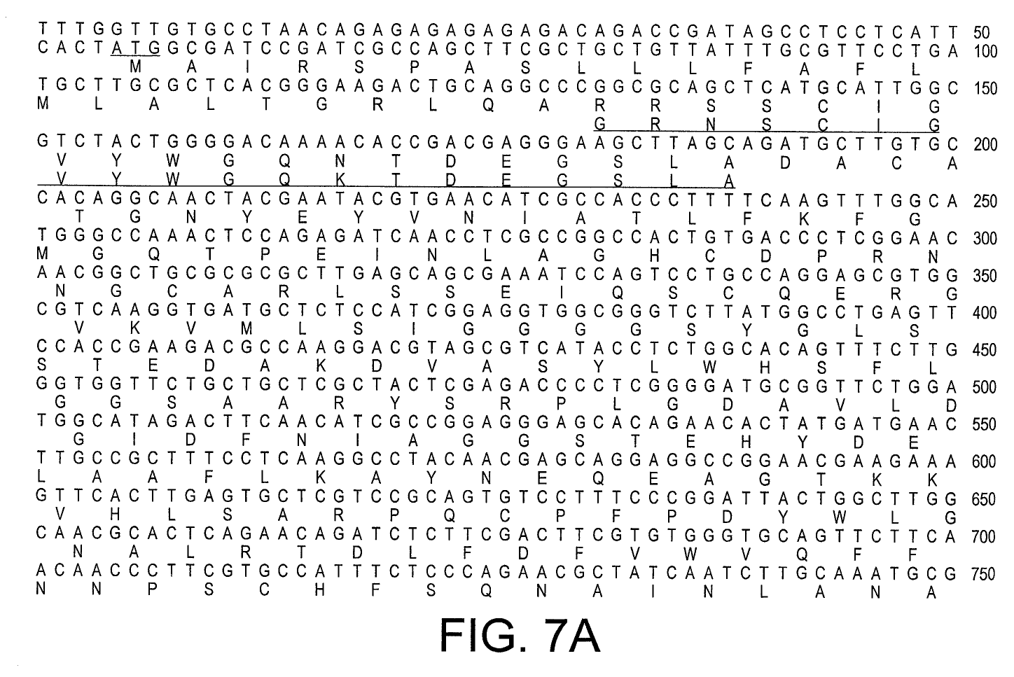

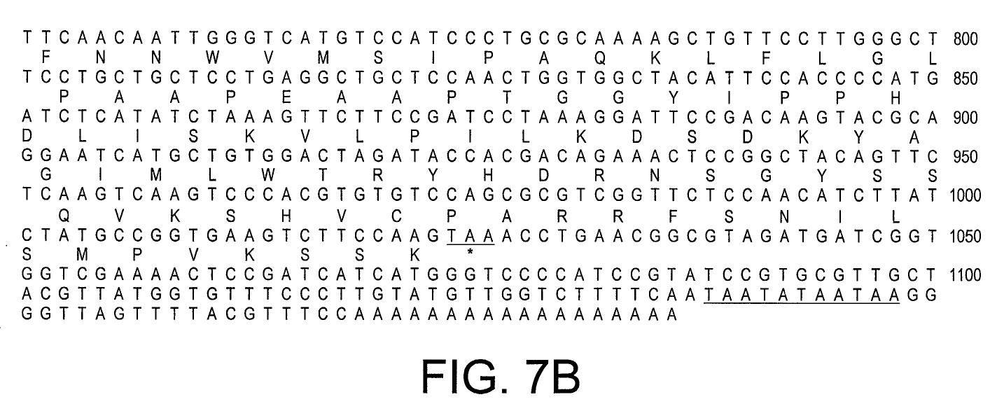

The N-terminus of the partially purified protein was sequenced and the resultant 20-amino acid sequence is: GRNSCIGVYWGQKTDEGSLA (data also appear in FIG. 7). A search of the amino acid sequence databhase (GenBank) revealed that the N-terminus of P31 shares significant homology to amino-terminal peptide sequences from purified acidic chitinases of Mongolian snake-gourd (Trichosanthes kirilowii; see Savary et al. (1994) Plant Physiol. 106:1195) and chick pea (Cicer arietinum; see, Vogelsgang, R., et al. (1993) Planta 189:60).

P31 Expression in Ripening Pulp

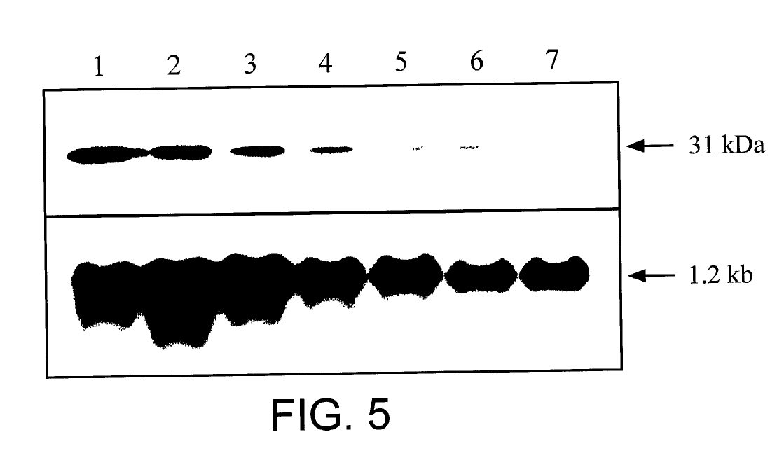

P31 expression in banana pulp during ripening was investigated at the protein and transcript levels. Western blot analysis of banana pulp proteins isolated at each of seven chronological stages of ripening (FIG. 5, top panel) indicates that P31 decreases in relative abundance during ripening, consistent with the observations of P31 abundance after separation by SDS-PAGE and staining with Coomassie blue. Using differential screening, several ripening-associated genes were isolated from a banana pulp cDNA library, including clones with significant homology to chitinases (Clendennen et al., supra). For determination of relative chitinase transcript abundance during ripening, total RNA was isolated from banana pulp during ripening, at PCI 1 through 7, and probed with labeled pBAN3-30. Northern blot analysis (FIG. 5, bottom panel) shows that the P31 message is strongly expressed ruing the first few ripening stages (PCI 1 through 3) after which the chitinase transcript declines in banana pulp through the later stages of ripening. This observation is consistent with the result obtained through western analysis. Northern and western blot analysis together suggest that expression of P31 is both fruit-specific and developmentally regulated in banana. While both the P31 protein and the chitinase transcript are abundant during the pre-climacteric stages of fruit ripening (PCI 1 through 3), their relative levels decrease as ripening progresses.

pBAN3-30 Encodes P31

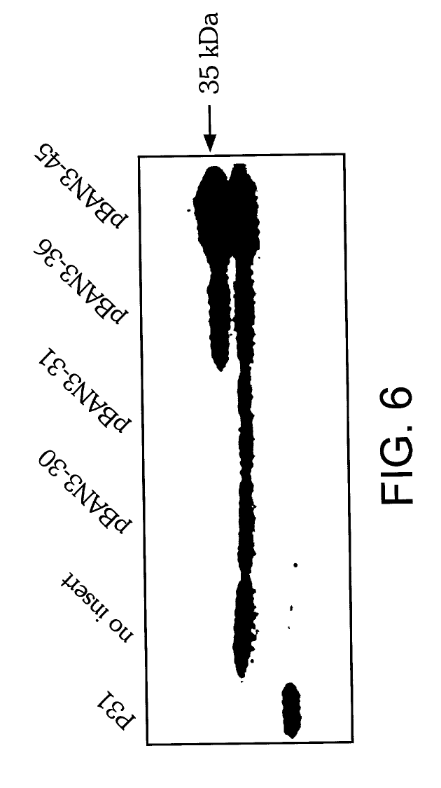

Three lines of evidence lead us to conclude that pBAN3-30 encodes the abundant 31 kDa pulp protein. First, the expression pattern of the pBAN3-30 transcript during ripening matches very closely with the profile of P31 abundance during ripening as determined by western blot analysis using the P31 antiserum, as seen in FIG. 5. Second, the P31 antiserum recognizes the translation product of the chitinase cDNA insert. The translation products of the cDNA clones pBAN3-36 and pBAN3-45, which are homologous to pBAN3-30 but have been determined to be in-frame with respect to the β-galactosidase gene in the pBluescript cloning vector (Stratagene), were expressed as fusion proteins with β-galactosidase. These fusion proteins were analyzed by western blotting and incubation with the P31 antiserum. The P31 antiserum recognizes a 35 kDa polypeptide produced in the IPTG-induced bacterial cells containing an in-frame chitinase cDNA (pBAN3-36 and pBAN3-45) that is not present in cell extracts from bacteria containing only the pBluescript plasmid (no insert) or out-of-frame chitinase cDNA inserts (pBAN3-30 and pBAN3-31) (FIG. 6). Finally, the N-terminal amino acid sequence obtained from the purified protein, which is underlined in FIG. 7, is identical to the deduced amino acid sequence of pBAN3-30 at 17 of 20 residues. This match is improved when the first amino acid residue, which is usually considered to be uncertain, is discounted. Despite the high sequence homology, the amino acid sequence from the partially purified protein is not completely identical to the amino acid sequence deduced from the cDNA clone pBAN3-30. It is possible that a contaminating polypeptide co-migrated with P31 and influenced the amino acid sequence results. Alternatively, it is possible that P31 is encoded by a gene family in banana, members of which are highly homologous, though not identical, and cannot be distinguished from one another by northern or western analyses.

Sequence Analysis of pBAN3-30

The complete nucleotide sequence of pBAN3-30 and the deduced amino acid sequence of the translation product is shown in FIG. 7. The cDNA insert is 1186 bp long and includes the entire chitinase coding region. The ATG beginning at position 55 is likely to be the translation initiation codon because the nucleotide sequence flanking the first ATG codon matches 8 of the 12 bases in the consensus for translation start sites in plants (Joshi, C. P. (1987) Nucl. Acids Res. 15:6543), whereas the sequences flanking another potential in-frame downstream start site (at position 100) is identical at only 5 of the 12 bases. There is an in-frame termination codon at position 1024 and several putative polyadenlyation signals between positions 1136 and 1148.

The open reading frame spans 323 amino acids from which a translation product of 35,232 Da is predicted. A GenBank search using the full cDNA sequence reveals significant homology between pBAN3-30 and chitinase genes characterized from winged bean (Psophocarpus tetragonolobus, M Esaka and T. Teramoto, unpublished), cow pea (Vigna unguiculata, L. T. T. Vo et al., unpublished), azuki bean (Vigna angularis; see, Ishige, F., et al. (1993) Plant Cell Physiol. 34:103), maize (Zea mays; see, Didierjean, L., et al. (1996) Planta 199: 1), and chick pea (Cicer arietinum; see, Vogelsgang, R., et al. (1993) Plant Physiol. 103:297). The deduced amino acid sequence of pBAN3-30 encoding P31 in banana shares sequence homology with other plant chitinases, especially with class III acidic chitinases that have been characterized from various dicots. At the amino acid level, the banana acidic chitinase amino acid sequence shows significant homology, 47-53% identity, to acidic chitinases from Arabidopsis thaliana (Samac, D. A., et al. (1990) Plant Physiol. 93:907), wine grape (Vitis vinifera, Busam et al, unpublished), tobacco (Nicotiana tabacum; see, Lawton, K. et al. (1992) Plant Molec. Biol. 19:735), chickpea, sugar beet (Beta vulgaris; see, Nielsen, K. K., et al. (1993) Molec. Plant-Microbe Interact. 6:495), winged bean, and cucumber (Cucumis sativus; see, Lawton, K. A. et al. (1994) Molec. Plant-Microbe Interact. 7:48).

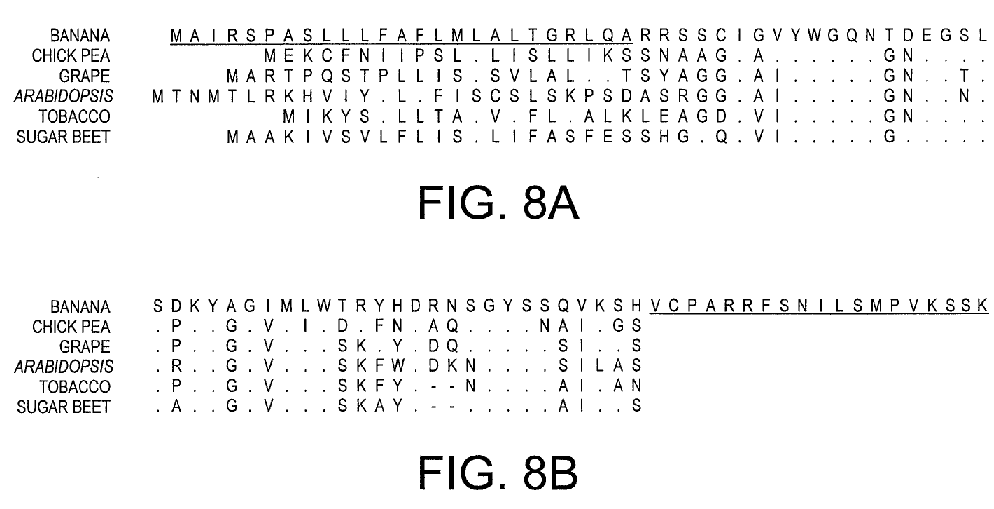

An amino acid sequence alignment of the amino-terminal and carboxy-terminal regions of several plant acid chitinases with P31 from banana appears in FIG. 8. Hydrophilicity analysis of the deduced protein sequence of P31 reveals a hydrophobic region from amino acid 1 to 25 (underlined in FIG. 8A). This region may represent a signal sequence that would direct targeting to the ER. If this putative signal peptide is removed, the remaining sequence closely matches the N-terminal sequence obtained from the purified protein, which suggests that P31 is post-translationally processed. This signal peptide does not share significant homology with the signal peptide sequences of other plant class III acidic chitinases (see FIG. 8A), which are typically localized to the extracellular space (Punja, Z. K. et al. (1993) J. Nematol. 25:526; Collinge, D. V., et al. (1993) Plant J. 3:31; Lawton, K. et al. (1992) Plant Molec. Biol. 19:735; Graham, L. S., et al. (1994) Canad. J. Botany 72:1057; Bol, J. F. (1990) Ann. Rev. Phytopathol. 28:113-138).

In addition to the N-terminal signal peptide, the banana P31 sequence is distinguished from other chitinase sequences by the presence of an 19 amino acid C-terminal extension (underlined in FIG. 8B). C-terminal propeptides (CTPPs) have been identified in a number of monocot and dicot polypeptides that direct proteins to the plant vacuole. Among others, CTPPs have been characterized in vacuolar lectins from barley and rice, and from vacuolar β-1,3-glucanase and chitinase from tobacco (see, Bednarel, S. Y. (1992) Plant Molec. Biol. 20:133, for review). In general there is little sequence homology among plant vacuolar targeting sequences. However, weak homology can be detected between the C-terminal extension of P31 (SNILSMP) and vacuolar targeting sequences that have been characterized in the sweet potato storage protein sporamin (NPIRLP) (Linthorst, H. J. M. (1991) Crit. Rev. Plant Sci 10: 123) and in a 2S albumin from Brazil nut (NLSPMRCP) (Saalbach, G. et al. (1996) Plant Physiol. 112:975).

Based on amino acid sequences, chitinases can be grouped into four classes. Class I includes a majority of chitinases described, possessing an N-terminal cysteine-rich lectin or “hevein” (chitin-binding) domain and a highly conserved catalytic domain. Class II chitinases lack the N-terminal cysteine-rich domain but have a high amino acid sequence identity to the main structure of class I chitinases. Class III chitinases show little sequence similarity to plant enzymes in class I or II, but may in fact be more similar to bacterial chitinases. The majority of class III chitinases are classified as such on the basis of homology to previously described lysozymes with chitinase activity. Class IV chitinases contain a cysteine-rich domain and conserved main structure which resemble those of class I chitinases by are significantly smaller due to four deletions (Punja, Z. K., et al. (1993) J. Nematol. 25:526; Collinge, D. V., et al. (1993) Plant J. 3:31; Graham, L. S., et al. (1994) Canad. J. Botany 72:1057). Although the banana pulp chitinase shares significant sequence homology with other plant class III acidic chitinases, the predicted isoelectric point of P31 is 7.63 (neutral). In addition, studies to determine the chitinase active sites in bacterial chitinases appear to be conserved in plant, bacterial, and fungal sequences (Perlick, A. M., et al. (1996) Plant Physiol. 110: 147). At least five highly conserved amino acids have been shown to be necessary for chitinase activity, and the deduced amino acid sequence of P31 indicates that only three of the five amino acids necessary for chitinase activity are conserved in banana P31 (not shown) (Watanabe, T., et al. (1993) J. Biol. Chem. 268:18567; Tsujibo, H., et al. (1993) Biosci. Biotech. Biochem. 57:1396).

Role of Chitinase in Banana Pulp

In plants, class III chitinases have been reported to be induced in response to various stresses such as pathogenesis and wounding (Ishige, F., et al. (1993) Plant Cell Physiol. 34:103; Lawton, K., et al. (1992) Plant Molec. Biol. 19:735; Nielsen, K. K., et al. (1993) Molec. Plant-Microbe Interact. 6:495; Lawton, K. A., et al. (1994) Molec. Plant-Microbe Interact. 7:48; Mehta, R. A., et al. (1991) Plant Cell Physiol. 32:1057). Recently, it has been reported that the expression of several pathogenesis and stress-related proteins, including chitinases, is associated with fruit ripening. Several genes encoding pathogenesis-related proteins such as endochitinase are associated with ripening in banana pulp (Clendennen, S. K., et al. (1997) Plant Physiol.). Considering the antifungal activity that they exhibit in other plants, it is possible that chitinases fulfill a protective role during fruit development and ripening. However, in contrast to the ripening-associated PR-proteins studied in cherry, avocado, and tomato, banana P31 decreases in abundance during ripening. Although it is possible that the banana chitinase serves a protective role during fruit development, an alternate hypothesis is that the chitinase in banana pulp has been recruited to serve as a storage protein in this tissue.

Storage proteins are a heterogeneous group of proteins for which no defined assay is available. According to a recent review (Staswick, P. E. (1994) Ann. Rev. Plant Physiol. Plant Molec. Biol. 45:303), storage proteins generally share the features listed below; we relate traits of P31 to general features of storage proteins.

1) Storage proteins are very abundant. We have found P31 to be very abundant in unripe banana pulp, accounting for approximately 20 to 30% of total soluble pulp protein. 2) Storage proteins are preferentially degraded during a subsequent developmental stage. For example, a vegetative storage protein characterized from the bark of poplar trees accumulates during fall and winter and is degraded during shoot growth in the spring. P31 is preferentially degraded during banana fruit ripening. Both the transcript and protein abundance decrease during ripening. If P31 is indeed a storage protein in banana pulp, this preferential degradation implies the existence of a protease specific to the storage protein, and inhibition of the protease would inhibit degradation of the storage protein. 3) Storage proteins are generally localized in storage vacuoles within the cell. The sub-cellular localization of P31 has not yet been determined. According to the deduced amino acid sequence of P31, there is a putative signal peptide sequence for P31 that is 25 amino acids long and hydrophobic. In addition, the amino acid sequence of P31 from banana pulp is distinguished from other plat class III acid chitinases by the presence of an 18 amino acid C-terminal extension that shows some homology to previously characterized C-terminal vacuolar targeting signals, suggesting vacuolar localization of P31. 4) Many storage proteins contain a large proportion of amino acid residues with nitrogen-containing R-groups. Amino acid composition analysis of P31 indicates that 22% of residues have N-containing R-groups (Trp, Gln, Asn, Lys, Arg, His). This is approximately the same proportion of N-containing R-group amino acids in vegetative storage proteins from soybean and poplar (21-25%). Interestingly, the N-containing R-group amino acid composition of P31 is not significantly higher than the N-containing R-group content of other plant chitinases (17-23%). 5) Storageproteins typically lack any other metabolic or structural role. However, this is not necessarily true for soybean vegetative storage protein, which has retained a minimal acid phosphatase activity, and patatin, a potato tuber storage protein that exhibits residual lipid acyl hydrolase activity. Preliminary evidence suggests that protein extracts from banana pulp have very low chitinase activity, as measured by soluble chitobiose released from radiolabeled chitin. In addition, only three of the five amino acids which have been determined to be essential for chitinase activity are conserved in P31. Taken together, this evidence lends support to the hypothesis that P31, while sharing sequence homology with plant chitinases, may actually be serving as a storage protein in banana pulp.

### EXAMPLE 3

### A Novel Fruit-Associated Class of Metallothionein-Like Proteins from Banana (Musa acuminata cv Grand nain): Characterization of the Gene Family and Induction by H₂O₂

In the experiments described in this Example, the gene family encoding the fruit-associated MTs is characterized, and sequence and functional evidence is provided that at least one member functions as an antioxidant during fruit ripening.

### Materials and Methods

Plant Materials

Ethylene treated and untreated banana fruit (Musa acuminata cv. Grand Nain) were obtained from the Northside Banana Company (Houston, Tex.). The pulp and peel of fruit representing different stages of ripening (PCI 1 and 3) were separated and quick-frozen in liquid nitrogen. Tissues from ten individual fruit were pooled to obtain a uniform representative sample for each ripening stage and ground to a fine powder under liquid nitrogen in a stainless steel Waring blender. Ground samples were stored at −80° C. until utilized. Leaf, corm and root tissue were obtained from greenhouse-grown plants (cv Grand Nain), ground in liquid nitrogen using a mortar and pestle, and stored at −80° C.

RNA Isolation and Northern Blotting

Pre-warmed (65° C.) RNA extraction buffer (1.4% (w/v) SDS, 2% (w/v) polyvinylpyrrolidone, 0.5 M NaCl, 0.1M sodium acetate, 0.05 M EDTA (pH 8.0) 0.1% (v/v) β-mercaptoethanol) was added to previously ground samples of pulp at a ratio of 5 ml buffer per gram of tissue. Samples were homogenized with several short bursts of a tissue homogenizer (Polytron, Brinkman) and incubated at 65° C. for 15 min. Starch and other cell debris were pelleted by centrifugation at 2,400 g for 10 min at room temperature and the supernatant transferred to a disposable polypropylene tube. After the addition of 0.2 vol. of 5 M potassium acetate (pH 4.8), the samples were mixed and incubated on ice for 30 min. The resulting precipitate was pelleted by centrifugation at 20,200 rpm for 10 min at 4° C. in a Sorvall SW28 rotor. The supernatant was transferred to a disposable polypropylene centrifuge tube, and the high-molecular weight RNAs were precipitated by the addition of lithium chloride to a final concentration of 2.5 M and incubation overnight at 4° C.

RNA was extracted from previously frozen ground peel, root, leaf, and corm tissue using CTAB extraction.

Five micrograms of total RNA from root, corm, and leaf tissue of greenhouse-grown plants, and from peel and pulp (PCI 3) were separated by electrophoresis in formaldehyde-containing 2% agarose gels and transferred to nylon membrane (Nytran Plust, Schleicher and Schuell) using 20×SSPE as a transfer buffer and a vacuum transfer apparatus (Bio-Rad). Equal RNA loading was confirmed by staining the RNA on the nylon membranes with methylene blue (Sambrook et al., 1989). RNA blots were prehybridized in 1 mM EDTA, 0.25 M phosphate buffer (pH 7.2), 7% (w/v) SDS, and hybridized overnight at 65° C. in the same solution containing the denatured probe (1×10⁷ cpm/ml). Hybridized filters were washed twice for 30 min each at 65° C. in Wash Solution One [1 mM EDTA, 40 mM phosphate buffer (pH 7.2) 5% (w/v) SDS] and three times for 30 min each at 65° C. in Wash Solution Two [1 mM EDTA, 40 mM phosphate buffer (pH 7.2), 1% (w/v) SDS]. The air-dried filters were subjected to autoradiography (X-Omat X-ray film, Kodak) at −80° C. with an intensifying screen. The RNA blots were hybridized with a cDNA probe representing either the MT cDNA clone isolated from library 1 or 3, using the Rad-Prime DNA Labeling System (Gibco BRL) to label the DNA probes.

Genomic DNA Isolation and Southern Blotting

Leaf tissue was ground with a mortar and pestle under liquid nitrogen and added to a tube containing pre-warmed (65° C.) DNA isolation buffer. The mixture was incubated at 65° C. for 30 minutes, then extracted twice with an equal volume of chloroform. After the second extraction, DNA was precipitated from the aqueous phase by the addition of an equal volume of isopropanol, and mixed by gently inverting the tube. DNA was pelleted by centrifugation, washed with 70% ethanol, dried briefly, and resuspended in TE (pH 8.0). DNA samples were treated with RNase, then phenol extracted with TE buffered phenol by rocking gently, chloroform extracted, and precipitated with 2.5 vol ethanol.

For the genomic Southern blots, 15 μg of genomic DNA was digested with restriction endonucleases BamHI, HinDIII, EcoRI, PstI, and SalI (Promega), and restriction fragments were separated by electrophoresis on a 0.7% agarose gel. DNA in the gel was denatured (1.5 M NaCl, 0.5 M NaOH) and neutralized (1.5 M NaCl, 0.5 M Tris, pH 8.0) before being transferred to nylon membrane (S&S Nytran Plus) using a BioRad vacuum transfer apparatus. DNA was covalently crosslinked to membrane by UV irradiation (Stratalinker, Stratagene), and the membrane was hybridized separately with probes corresponding to the MT cDNA clones isolated from the banana pulp cDNA libraries from PCI 1 and 3 (MT-F1 and MT-F3).

Genomic Library Screening and Mapping

Approximately 6×10⁵ primary plaques from a Musa acuminata cv Grand Nain λ FIX genomic library (Stratagene) were screened with the MT cDNA probe isolated from the PCI pulp cDNA library (MT-F1). Plaque-lifts containing filter-bound λphage DNA was denatured for two min in 1.5 M NaCl, 0.5 M NaOH, and neutralized for four minutes in 1.5 M NaCl, 0.5 M Tris (pH 8.0). Filters were rinsed in 0.5 M Tris (pH 8.0), blotted dry, and the DNA was covalently crosslinked to the filters by UV irradiation (Stratalinker, Stratagene). Plaque-lifts were hybridized as described previously. Twenty-four positives were plaque purified, and λphage DNA was isolated for generating maps of the region containing the MT gene. Southern blot analysis was used to determine the identity of the MT clone according to diagnostic restriction sites. Fragments of the genomic clones containing the coding region and 5′ and 3′ flanking region were subcloned into pBluescript KS, and subclones were mapped and sequenced.

Sequencing and Data Analysis

Small-scale alkaline lysis plasmid preparations followed by phenol:chloroform extraction and ethanol precipitation (Sambrook et al., 1989) yielded template plasmid DNA suitable for automated sequencing. Plasmid DNA templates were sequenced, using the T3 primer, on an ABI 373A DNA sequencer (Applied Biosystems, Foster City, Calif.).

Using the BLASTX search algorithm, it was determined that the banana cDNA clones shared significant sequence homology with MT cDNA clones isolated from other fruit. The deduced amino acid sequences of plant MT cDNA clones were aligned using Clustal. A dendrogram showing the relationship among several different classes of plat MTs was generated from the deduced amino acid sequences using Clustal.

Protoplast Isolation and Dot Blot Analysis of MT Transcript Abundance

Protoplasts from banana pulp at PCI 4 were isolated as described in Khalid et al. (in preparation). 1×10⁵ protoplasts were incubated under experimental conditions for 4 h at room temperature in protoplast isolation buffer (Khalid et al. 1997), with gentle rocking to keep the cells suspended. The treatments included incubation with different concentrations of ascorbate (buffered to pH 7.0), CuCl₂, and hydrogen peroxide from 1 to 100 mM. After the incubation, a crude RNA preparation from the protoplasts was spotted onto nylon membrane in duplicate. One membrane was hybridized to the F3 cDNA probe to determine relative transcript abundance of fruit-associated MT. The second membrane was hybridized with an 18S ribosomal RNA probe to assess RNA loading. The membranes were then exposed to a phosphorescent screen (PhosphorImager, Molecular Dynamics) and the scanned images were quantified with the ImageQuant software. The relative abundance was normalized to the measure of total RNA loaded, and is expressed in arbitrary units.

### Results

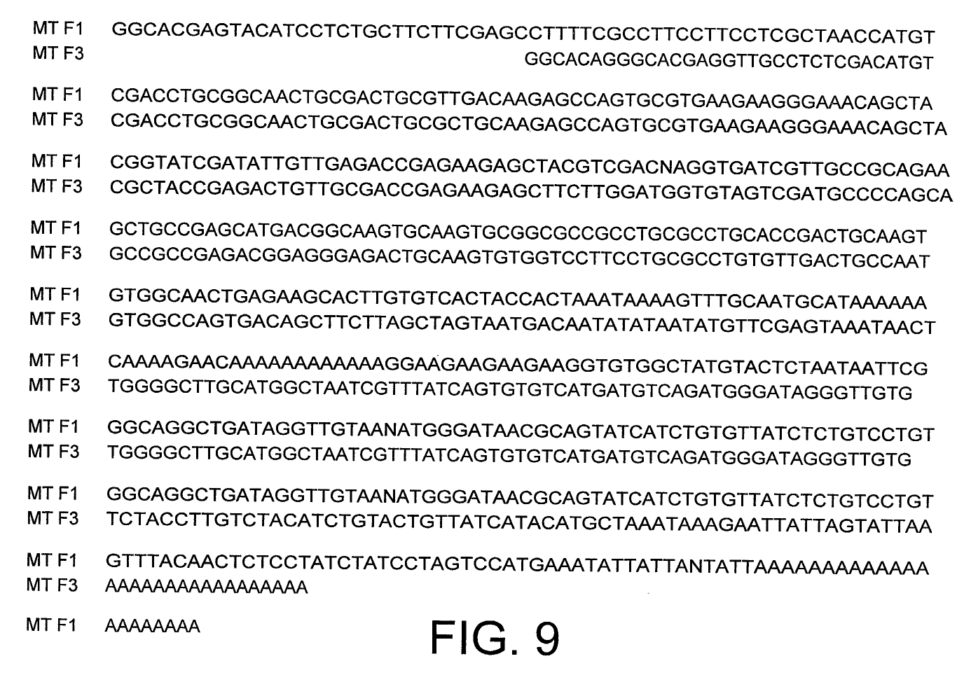

The cDNA sequence of the banana fruit-associated MT clones is shown in FIG. 9. The clones were isolated by differential screening of pulp cDNA libraries (Clendennen and May, 1997). F1 was isolated from the PCI1 library, whereas F3 was isolated from the PCI3 library. The cDNA clones are slightly variable in size, and most of the differences in size and primary sequence occurs in the 3′ untranslated region (UTR), with F1 having approximately 70 more bases than F3. The two banana cDNA sequences are 60% identical at the nucleotide level, and 81% identical within the coding region.

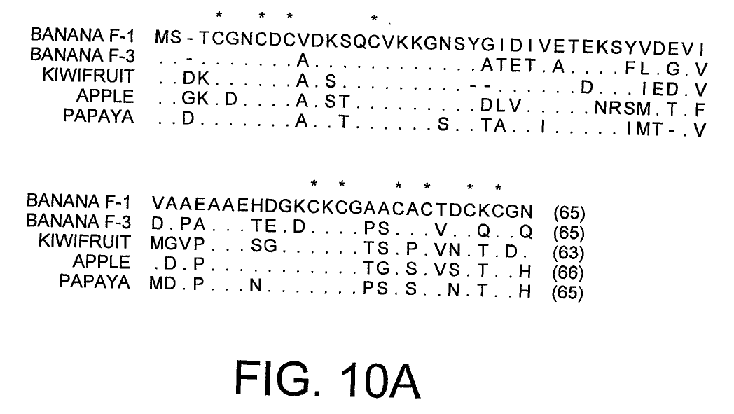

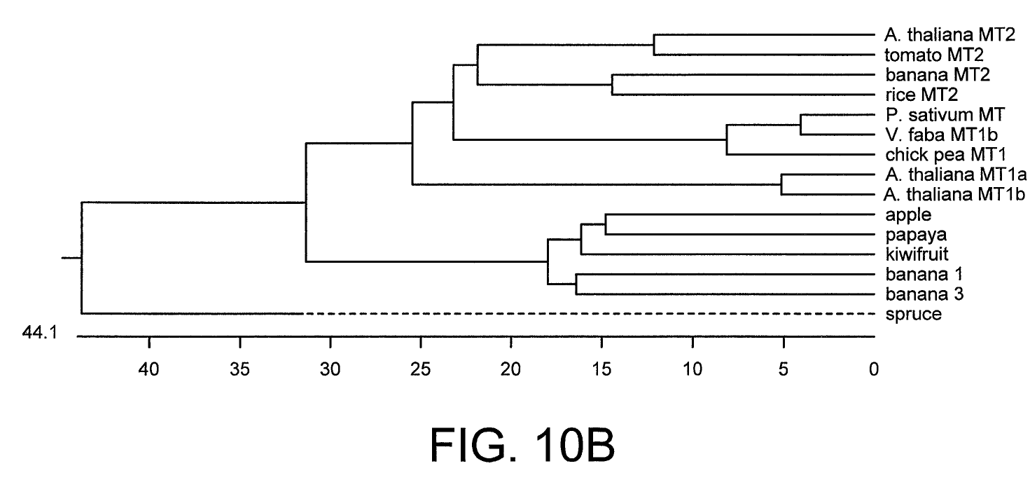

While both of the banana fruit-associated MT polypeptides are 65 amino acids, the two cDNA clones encode distinct polypeptides. The N-terminal and C-terminal domains are well conserved, and separated by a variable spacer. In FIG. 10A, an alignment of deduced amino acid sequences shows the degree of similarity among the different fruit-associated MTs from banana, kiwifruit, papaya, and apple. In panel B, the relationships among a variety of plant MTs is depicted in a dendrogram generated from a cluster together, as do the type 1 MT sequences. The fruit-associated MT sequences (banana, kiwifruit, papaya, and apple) cluster together and are distinct from both type I and type 2 plant MTs.

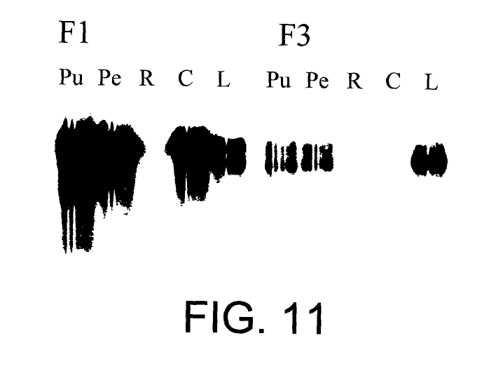

Despite the sequence similarity, the size difference between the transcripts of the two banana MT cDNA clones allows them to be separated on a high percentage (2%) agarose gel and detected by northern blotting and hybridization separately with each probe (FIG. 11). Transcript sizes of F1 and F3 as determined from northern analysis are approximately 540 and 430 bases, respectively. The larger transcript (F1) is abundant in pulp, peel, and corm. It is also present in low abundance in banana leaves, but is not detected in roots. The smaller transcript (F3) is most abundant in leaves, present in pulp and peel, and barely detectable in root and corm tissue.

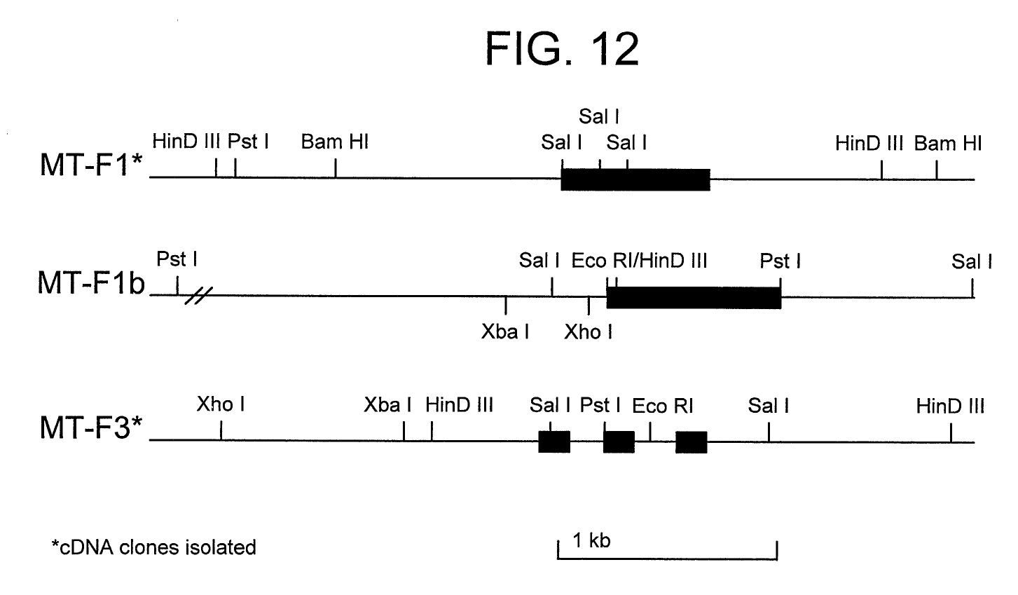

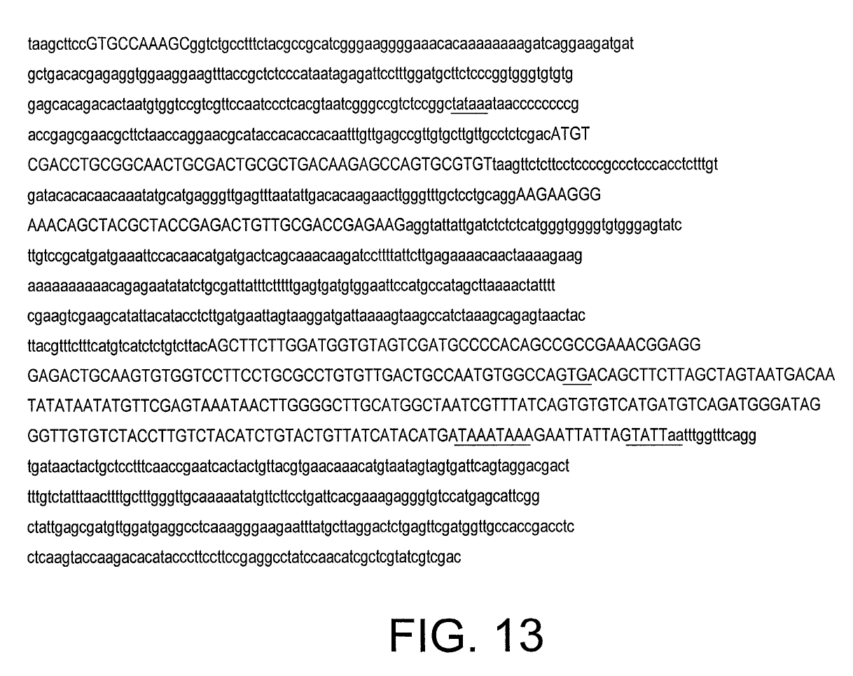

Southern analysis using both cDNAs as probes indicates the presence of up to five copies of the fruit type MT—two copies with homology to F1 and three copies with homology to F3 (data not shown). Twenty-four genomic clones of fruit MT were isolated from the genomic library, and restriction maps of the region containing the MT gene indicated that three distinct genes had been isolated. Clones representing both the F1 and F3 cDNA clones were isolated from the genomic library, as well as a gene with homology to the fruit-associated MT F!, but for which no cDNA clone has been isolated (named MT-F1b). Subclones of these different MT genes were generated and the region containing the coding region as well as 5′ and 3′ flanking regions were mapped. Maps of the different MT genes, including the coding region and at least 1 kb of 5′ and 3′ flanking regions appear in FIG. 12. Based on mapping and sequence data it can be determined that the MT F3 gene is comprised of three exons separated by two introns. The mapping resolution was not fine enough to determine the existence or position of introns in the other MT genes. The nucleotide sequence of the F3 genomic clone from the HindIII site to the SalI site, which includes the complete coding region, is depicted in FIG. 13. Several features of the sequence are highlighted in the figure, including a 10-base 5′ sequence motif beginning at −313 from the translation start site (in capital letters) that shares homology with an antioxidant response element. The putative TATA-box (starting at position −96 from the translation start site) is underlined, and the three exons (beginning from the translation start site) are depicted in capital letters. At the 3′ end of the sequence, the stop codon is underlined, as well as a potential polyadenylation signal (TAAATAAA).

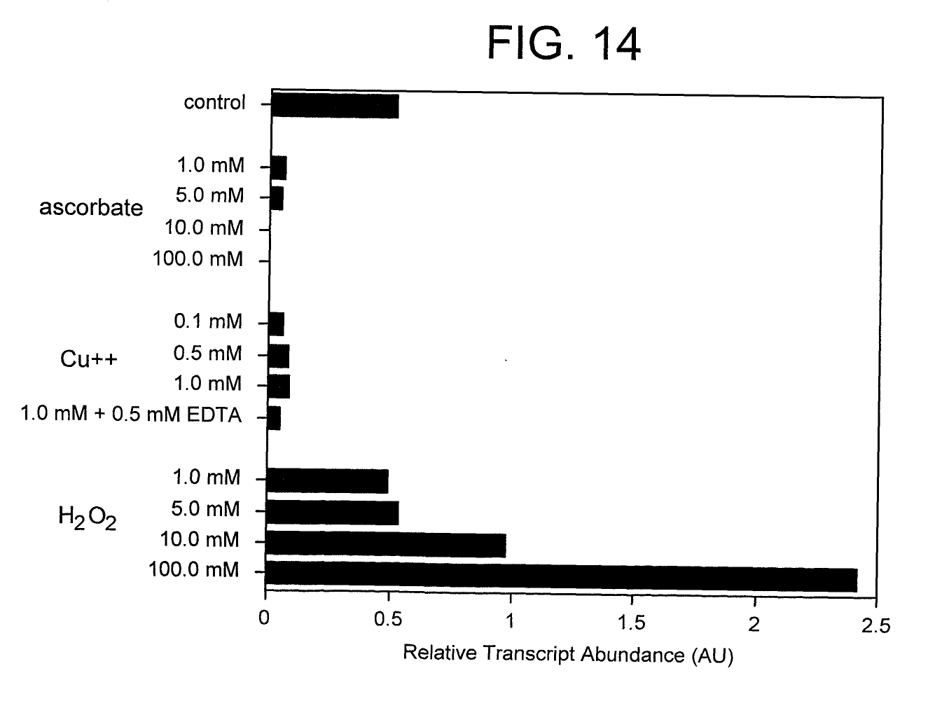

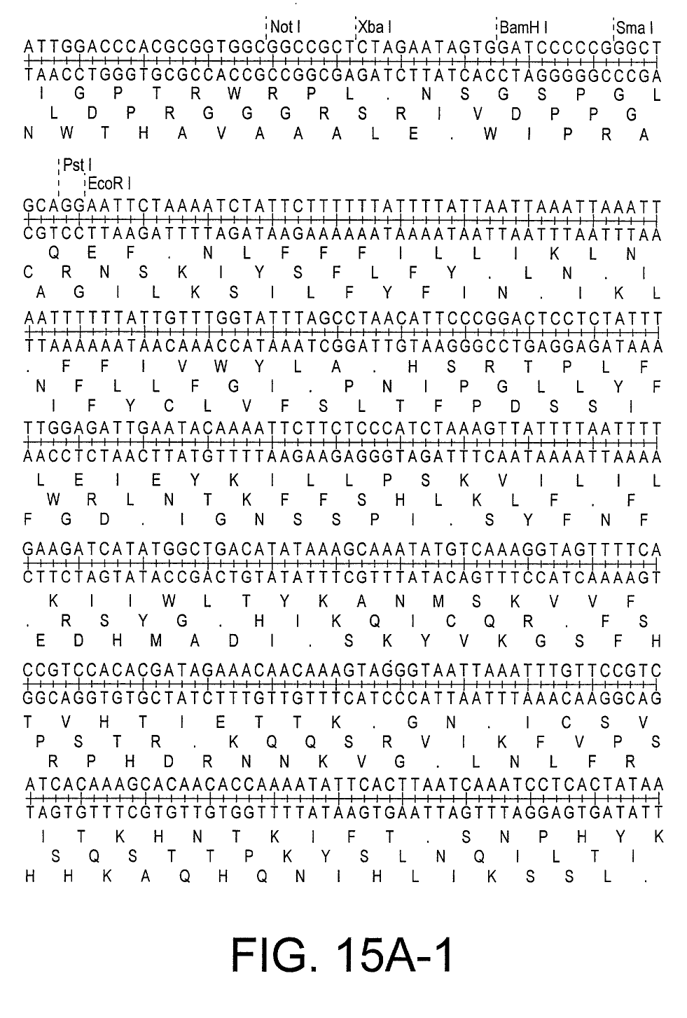

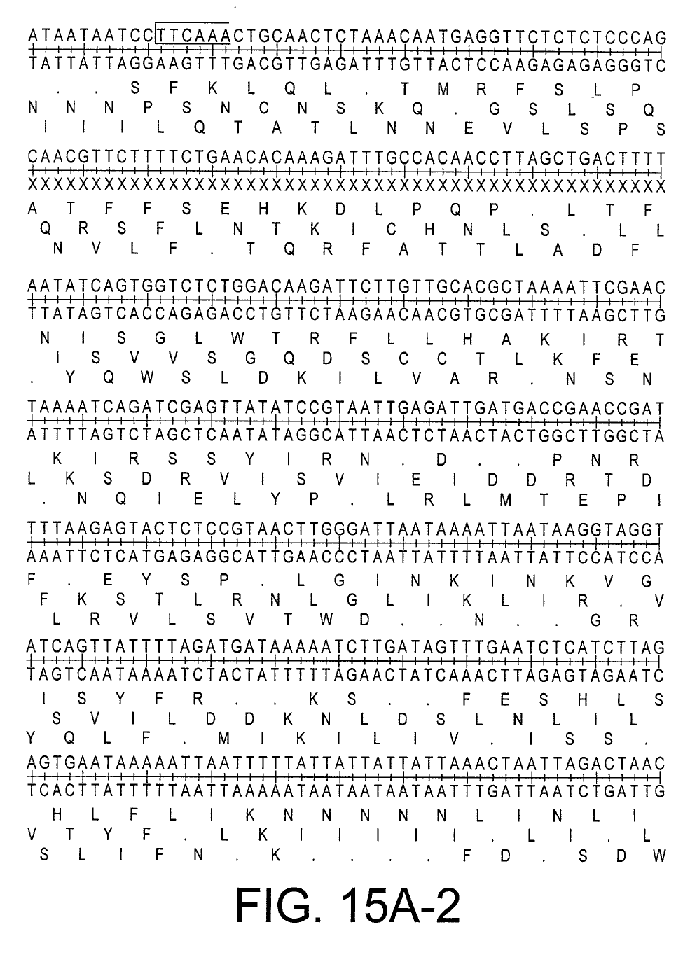

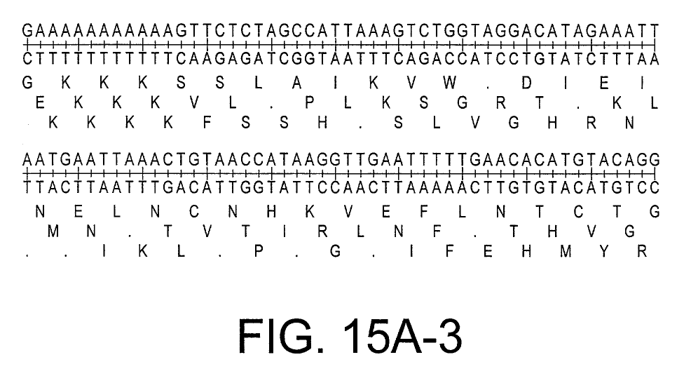

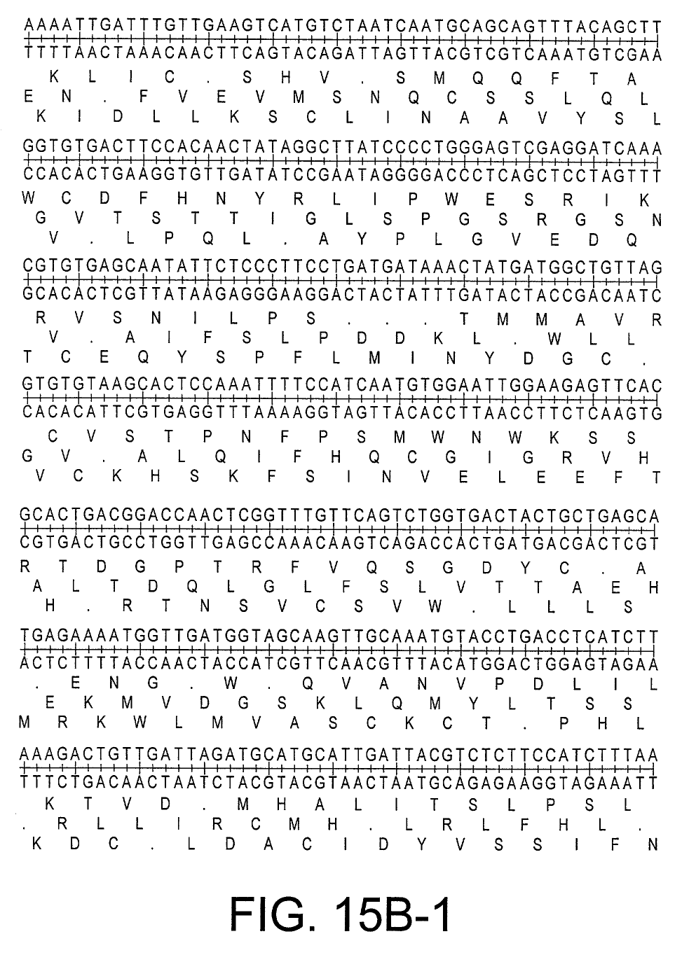

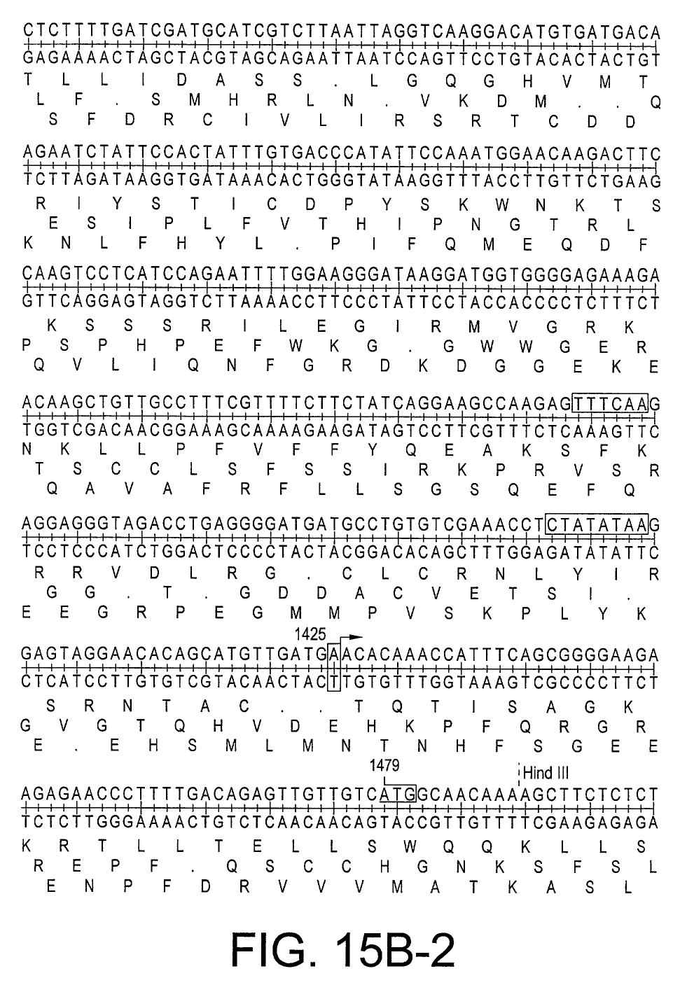

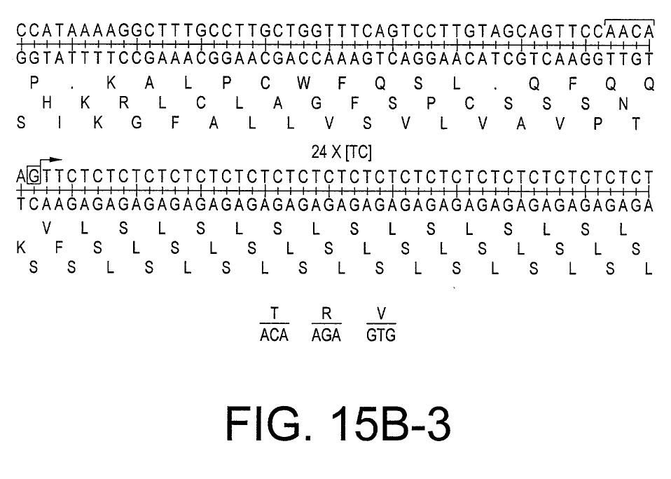

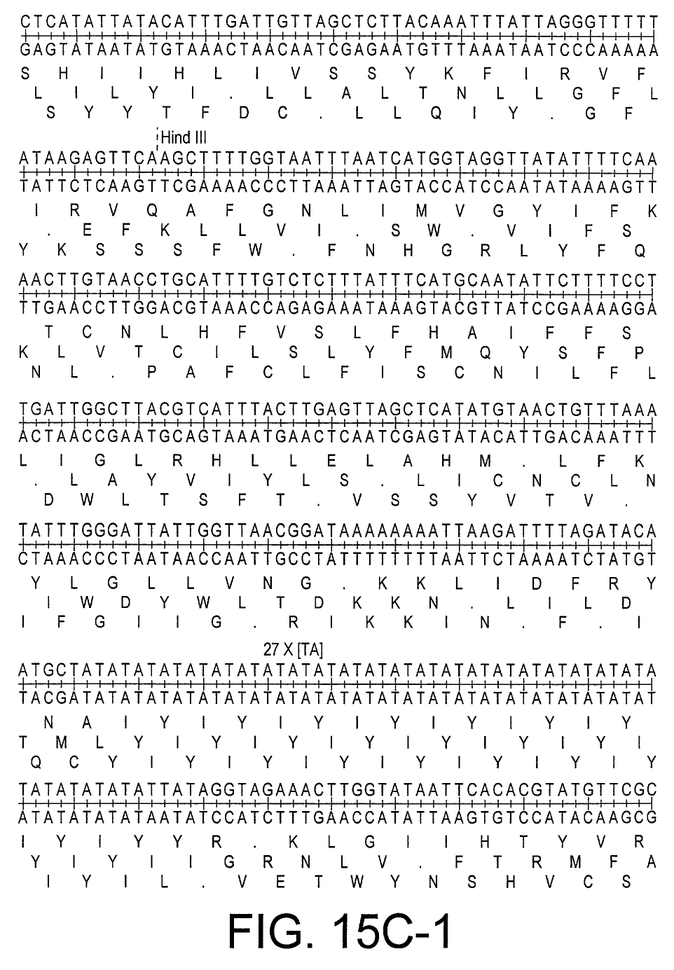

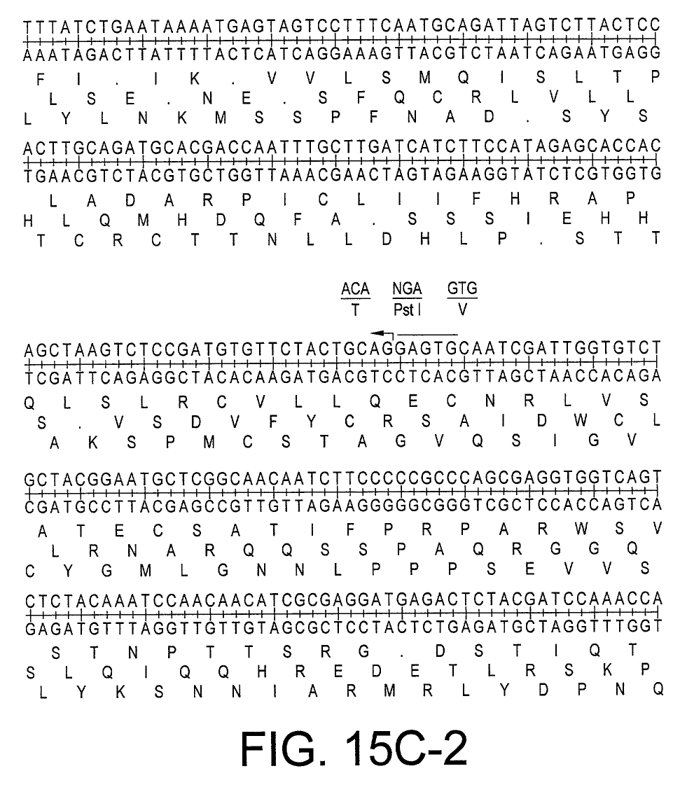

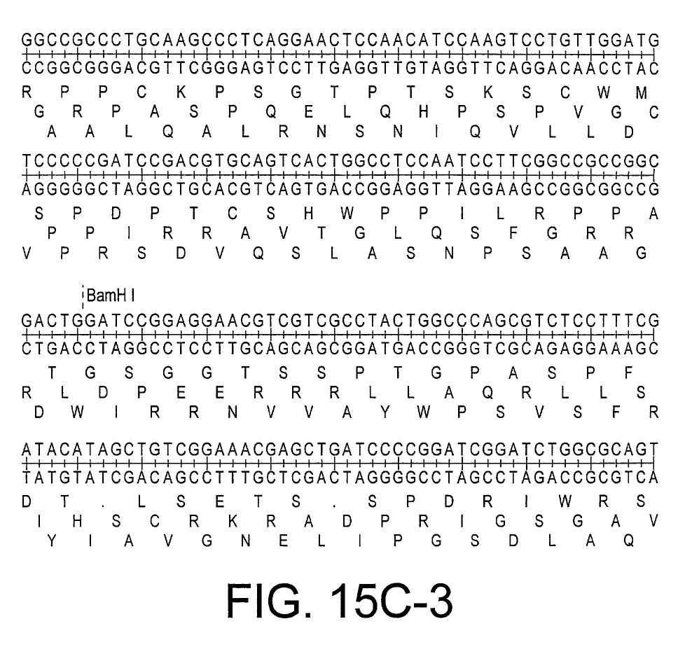

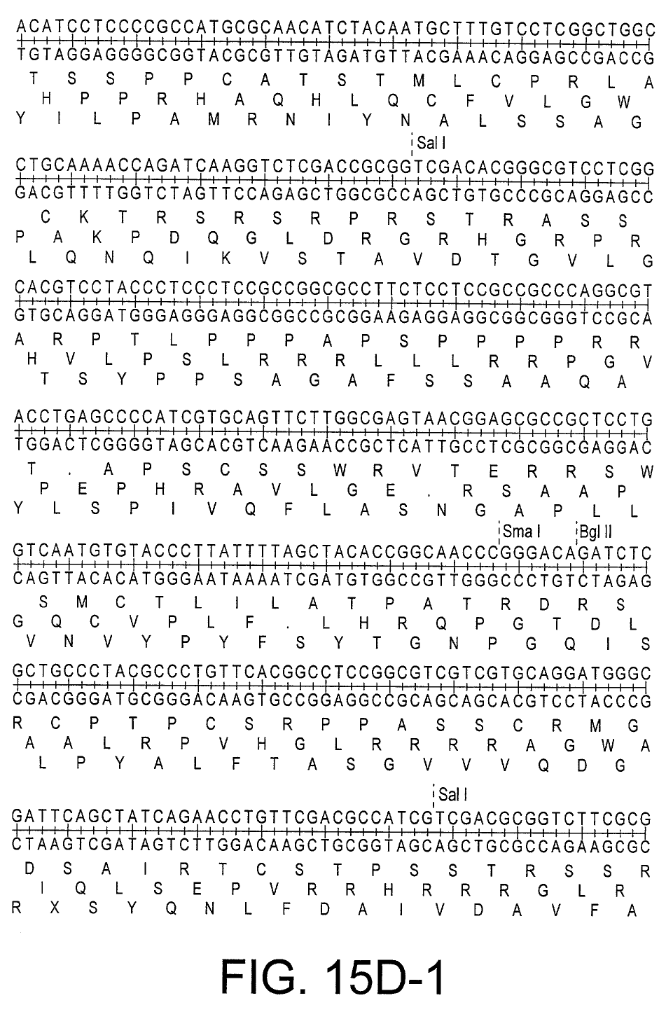

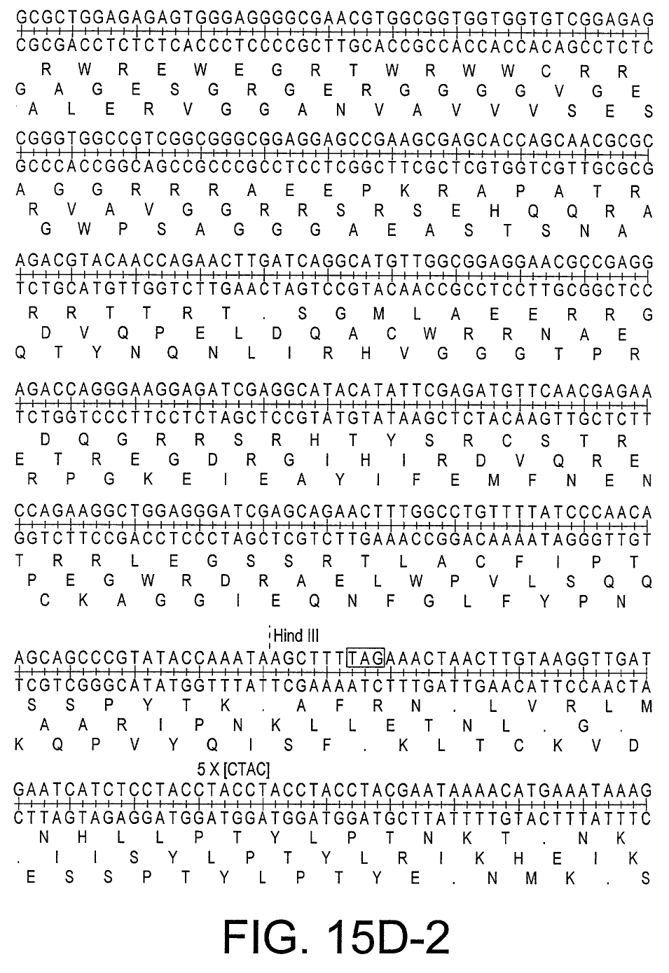

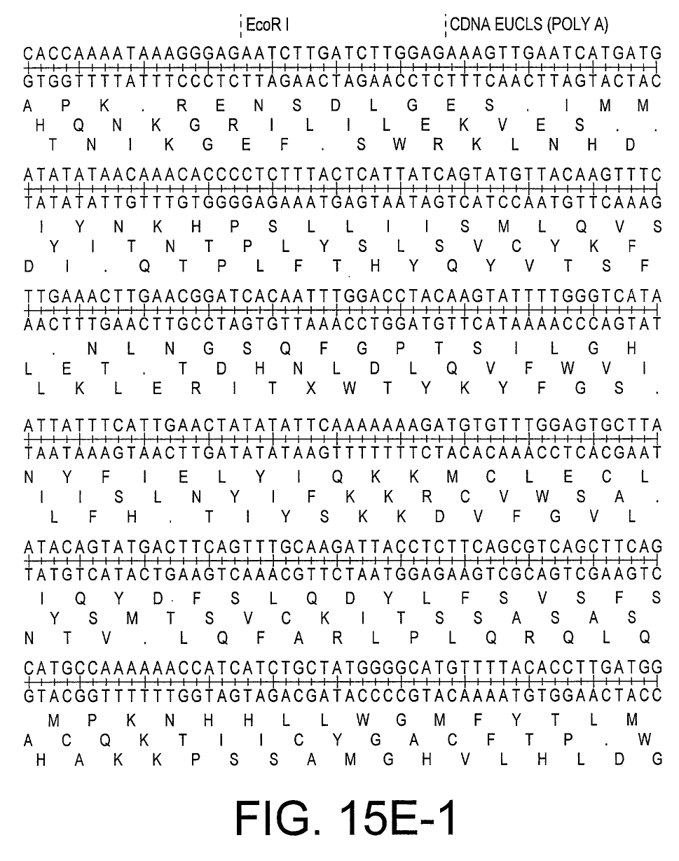

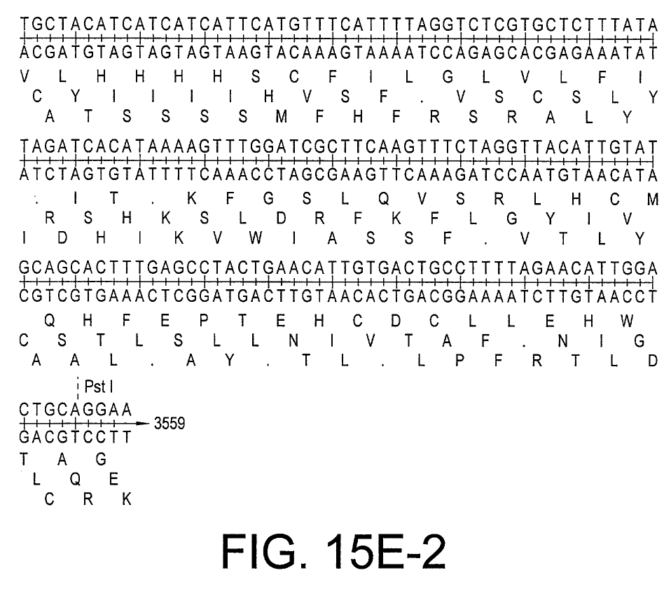

Because of the putative ARE identified in the 5′ flanking sequence, the effect of antioxidants (ascorbate), oxidizing agents (H₂O₂), and metal ions (Cu⁺⁺) on MT transcript abundance was determined in banana pulp protoplasts. H₂O₂, but not copper ions, resulted in dramatic and dose-dependent increase in the relative abundance of the fruit-associated MT transcript (FIG. 14). The presence of ascorbate resulted in a reduction in the relative MT transcript abundance as compared to an untreated control.

### Discussion

Eleven non-redundant groups of ripening-associated cDNA clones were isolated from banana pulp cDNA libraries by differential screening and identified by sequence homology (Clendennen and May, 1997). One of the groups of cDNA clones includes a previously uncharacterized type of metallothionein (MT), the transcript of which is found abundantly in ripening banana pulp. There are two classes of this ripening-associated MT transcript in banana pulp that vary in primary sequence and in size. Both the larger (F1) and the smaller (F3) transcripts increase in abundance in banana pulp during ripening, but F1 increases more dramatically than F3. In addition, the tissue distribution of these transcripts differs: MT-F1 is expressed abundantly in the pulp and peel, and slightly in corm tissue, whereas MT-F3 is expressed abundantly in pulp, peel, and leaves, and very slightly in roots. Based on the isolation of two distinct cDNA clones, it was suspected that the fruit-associated MTs were encoded by a small gene family. Southern analysis confirmed this, and suggested the presence of up to five members of the fruit-associated MT gene family in banana. Three different MT genes were identified after screening twenty-four genomic clones that hybridized to F1 and F3 cDNA probes, as determined by restriction mapping of the segment containing the coding region. Genomic clones representing both cDNA clones were isolated.

### EXAMPLE 4

### Demonstration of Functional Banana p31 Promoter-Driven Heterologous Gene Expression in Tomato

In the experiments described in this Example, the p31 promoter is inserted in a plant transformation vector with a reporter gene (β-glucuronidase), which is used to transform tomato plants, providing functional evidence that the p31 promoter derived from banana is capable of driving heterologous gene expression in a dicotyledonous fruiting plant, tomato.

### Materials and Methods

Banana p31 Promoter Expression Cassette Construction

A 4.85 kb BamHI/SalI banana genomic fragment, containing the entire coding sequence of the banana p31 protein and 2.15 kb of 5′ flanking region containing the putative promoter having nucleotide sequence SEQ ID NO: 44 (SEQ ID NO: 44: GGATCCCAACTTTTAGGAATGGATCTTAAAATTTTAGTTATAAGTT CAAAGTTAGAAAAATCTTTACCAAGAGCTTTGAGTCCATTGATGACATCCGTGA AACGGTGTACATGTCTCCGATGGACTCACTTGGTTTCATTCGGAAAAGTTCGAA AGAGTGCATAAGAATATTGATTTTGGATTCTTTCACTCGGTTGGTGCCTTCATG AGTGACCTCAAGAGTCCTCCAAATATCAAAAGCCGAATCACAAATTGAAATGT GATTGAATTCATTTTTGTCTAATGCACAAAACAGGGCATTCATAGCCTTTGTGT TTAAAGCAAAAACATTCTTCTCCGATTCATCCCATTCGCTCATCGGAAGAGAAA ATTTTTGAAATCCATTTTCGACAATAGACCAAAGCTCGAAATCCATGGAAATGA GGAAGATCCTCATATGAGTTTTCCAATACATGTAATTCGACTCATTAAACATAG GTGGATGTGTAATGAAATGACCCTCATGCSCTATCTCTCTTGGGTATTAAACCA AATATGAGAGTGAGCCTTGCTCTGATACCAATTGTTAGGATCAGAGTGGCACTA AGAGAGGGGGGGAGTGAATTAGTGCAGTGGATTAAAACTTATAAGTTTAAAAA TGAATTCGTAAATACGAGAAGATTTCGTTTTAATAGTAACTTGAGTAGATGAAA ACCAAAAGTTAACAGTAGTGTAAATAACAATTTCGGGAAAGTAAGAACTCACA CATTCAAGGAACATACCAATTTAAAGTGGTTCGGTCAAAATGACCTACATCCAC TTGTGAAGCCTTCTTCGAAGAGGCTCCCAACTTCCACTAGCAAATCACTTTGAA GGGGAAGGACAAATACCTCTCTTACNACCTTTTACAATGGTTCATACTCTTACA AATTTTCAACGAGAAAGAAGGAGGTGAACATGCAAGCAATTGAAAACAAGACT TGCTAAAGACTTTGCTAAGGCTTTTTTTCTCAATCTATTGCTTCTCAAAAGTTGT ATTCTCTGCTGAGAATTGAGGGGTATTTATAGACCCCAAGAGGATTTAAATTTG GGCTCCAAATTTCGAATGCTCTTGGGTTCCCGAGGTTGCCGGTGCCACCGCCTG TCAGTGTTTGACACTGGACAGTGTACTAGCGGTGCCACCGCCGGACCTCTCGGG TGTTGGGCGGTGCCACCGCCTAGACTTTTTCAGCTCACTGGTTGGATTCCAAAC TTGACCCAAACCAGTCCGAACTCGGGTCCAATTGACCCGTAACCGGATTATAGG ATTAACCCTTAATCCTAACCCTAATTATATGCAAACTACGCAACTGAAAATATA GTCCTAAGCAAGTTTTTAACCGGCAAACGTCGAGTCTTCTTCCGGCGATCTTTC GGCAGACTTCTGATATACCTTTGGATTTCTTCTAGCGGACTCCTAGTAGGGTCC CGATCTTGTGGCGAGTTTAGCGAGTAGCCGAACCTTCTCGGTGATCTCCGCAAA CCGCCGATGATCTCTTCGGCAGACTTTCGAAAACTTCGACAAGTCCCCGATTTC TTCTCGGTTGGTTCCGACAGCATCTCTAACGAAACTTCGGACTCCTTGAATGTC CATCGAACTTGACTCCGGTAGGCTTGCTTTATATTTTCAGGCTATCATAGTTAAT CCTACATACTTAACTCAATAATATGGATTAGATTAATTAACCCATCAATTGATT TCATCATCAAAATTCGACATTCAACAAACATCCGTACTCAATAACCCATCAGGC TATAGTTACGTGACTATCTACTGTGATCCGTACGTGAAGTTAGCGAGTCATGAT CCAGGTCGTGTCACTTATTGGCCGAACACGTATCCCTTATCCAAATCCAGTCTT CTCAACTCTTCTAGCCTACCCGTCTCTTTTTTTATTACTTTTGAAAGAATTCAAA TCAAAACAGATACAAAATAACACGGTGAGACACTGTGACATGCTAGTCTCTGG AAAGCATTAATTCGCGCATCCACAGACGTCGTCAGCTTCATCACCCACTTTTTC CTACATACCATGTCGCATGGCTTTGTTGATGACAGACCACCACAAGCTTGCCTT TGGTTGTGCCTAACAGAGAGAGAGAGAGAGACAGACCGATAGCCTCCTCATTC ACTATGG), was subcloned from a Lambda-FIX® II library (Stratagene) into pBluescriptII-SK (Stratagene) to create pBS-31. A NcoI site at position −1741 relative to the start codon was removed by digestion of pBS-31 with NcoI, filling the ends with Klenow enzyme, and religating to form p31!N. A new NcoI site was created spanning the translation start site by PCR with template p31!N and the mutagenic primer p31-Nco (5′-GATCGCCATGGTGAATG) (SEQ ID NO:42) with the M13F primer (5′-GTAAAACGACGGCCAGT) (SEQ ID NO:43), performing 25 cycles of 94° C. for 45 seconds, 46° C. for 45 seconds and 72° C. for 60 seconds. The 2.1 kb product with nucleotide sequence SEQ ID NO: 45 (SEQ ID NO: 45: GGATCCCAACTTTTAGGAATGGATCTTAAAATTTTAGTTATAAGTT CAAAGTTAGAAAAATCTTTACCAAGAGCTTTGAGTCCATTGATGACATCCGTGA AACGGTGTACATGTCTCCGATGGACTCACTTGGTTTCATTCGGAAAAGTTCGAA AGAGTGCATAAGAATATTGATTTTGGATTCTTTCACTCGGTTGGTGCCTTCATG AGTGACCTCAAGAGTCCTCCAAATATCAAAAGCCGAATCACAAATTGAAATGT GATTGAATTCATTTTTGTCTAATGCACAAAACAGGGCATTCATAGCCTTTGTGT TTAAAGCAAAAACATTCTTCTCCGATTCATCCCATTCGCTCATCGGAAGAGAAA ATTTTTGAAATCCATTTTCGACAATAGACCAAAGCTCGAAATCCATGCATGGAA ATGAGGAAGATCCTCATATGAGTTTTCCAATACATGTAATTCGACTCATTAAAC ATAGGTGGATGTGTAATGAAATGACCCTCATGCSCTATCTCTCTTGGGTATTAA ACCAAATATGAGAGTGAGCCTTGCTCTGATACCAATTGTTAGGATCAGAGTGGC ACTAAGAGAGGGGGGGAGTGAATTAGTGCAGTGGATTAAAACTTATAAGTTTA AAAATGAATTCGTAAATACGAGAAGATTTCGTTTTAATAGTAACTTGAGTAGAT GAAAACCAAAAGTTAACAGTAGTGTAAATAACAATTTCGGGAAAGTAAGAACT CACACATTCAAGGAACATACCAATTTAAAGTGGTTCGGTCAAAATGACCTACAT CCACTTGTGAAGCCTTCTTCGAAGAGGCTCCCAACTTCCACTAGCAAATCACTT TGAAGGGGAAGGACAAATACCTCTCTTACNACCTTTTACAATGGTTCATACTCT TACAAATTTTCAACGAGAAAGAAGGAGGTGAACATGCAAGCAATTGAAAACAA GACTTGCTAAAGACTTTGCTAAGGCTTTTTTTCTCAATCTATTGCTTCTCAAAAG TTGTATTCTCTGCTGAGAATTGAGGGGTATTTATAGACCCCAAGAGGATTTAAA TTTGGGCTCCAAATTTCGAATGCTCTTGGGTTCCCGAGGTTGCCGGTGCCACCG CCTGTCAGTGTTTGACACTGGACAGTGTACTAGCGGTGCCACCGCCGGACCTCT CGGGTGTTGGGCGGTGCCACCGCCTAGACTTTTTCAGCTCACTGGTTGGATTCC AAACTTGACCCAAACCAGTCCGAACTCGGGTCCAATTGACCCGTAACCGGATTA TAGGATTAACCCTTAATCCTAACCCTAATTATATGCAAACTACGCAACTGAAAA TATAGTCCTAAGCAAGTTTTTAACCGGCAAACGTCGAGTCTTCTTCCGGCGATC TTTCGGCAGACTTCTGATATACCTTTGGATTTCTTCTAGCGGACTCCTAGTAGGG TCCCGATCTTGTGGCGAGTTTAGCGAGTAGCCGAACCTTCTCGGTGATCTCCGC AAACCGCCGATGATCTCTTCGGCAGACTTTCGAAAACTTCGACAAGTCCCCGAT TTCTTCTCGGTTGGTTCCGACAGCATCTCTAACGAAACTTCGGACTCCTTGAAT GTCCATCGAACTTGACTCCGGTAGGCTTGCTTTATATTTTCAGGCTATCATAGTT AATCCTACATACTTAACTCAATAATATGGATTAGATTAATTAACCCATCAATTG ATTTCATCATCAAAATTCGACATTCAACAAACATCCGTACTCAATAACCCATCA GGCTATAGTTACGTGACTATCTACTGTGATCCGTACGTGAAGTTAGCGAGTCAT GATCCAGGTCGTGTCACTTATTGGCCGAACACGTATCCCTTATCCAAATCCAGT CTTCTCAACTCTTCTAGCCTACCCGTCTCTTTTTTTATTACTTTTGAAAGAATTC AAATCAAAACAGATACAAAATAACACGGTGAGACACTGTGACATGCTAGTCTC TGGAAAGCATTAATTCGCGCATCCACAGACGTCGTCAGCTTCATCACCCACTTT TTCCTACATACCATGTCGCATGGCTTTGTTGATGACAGACCACCACAAGCTTGC CTTTGGTTGTGCCTAACAGAGAGAGAGAGAGAGACAGACCGATAGCCTCCTCA TTCACCATGG) was gel-purified and ligated with T-tailed pBluescriptKS (Stratagene). The T-tailed pBluescriptKS was prepared by digesting the plasmid with EcoRV and treating with Taq polymerase and dTTP at 72° C. for two hours. A clone was selected and named pKS-31Nm, and the sequence surrounding and upstream of the newly created NcoI site was confirmed by DNA sequencing. The 3′ end of the p31 promoter contained in pKS-31Nm was obtained by digestion with PacI and NcoI. This 454 bp fragment of SEQ ID NO: 45 was ligated with the 1874 bp SacI/PacI fragment from p31!N and NcoI/PacI-digested vector pGEM5-zf (Promega) to form pGEM-31N!N.

Since DNA sequencing of the 3′ end of the p31 promoter in pKS-31Nm detected a single “T” insertion 5′ of the HindIII site at position −103 relative to the start codon, only the short fragment HindIII/NcoI was used. An intermediate vector, pKS-31TH, was constructed by ligation of the 2158 bp BamHI/NcoI fragment of pGEM-31N!N with the 956 bp NcoI/EcoRI fragment of pTH210 (Haq, et al. (1995) Science 268:714-716) and the BamHI/EcoRI-digested vector pBluescriptKS. Ligation of the 448 bp HindIII/Kpn1 fragment of pKS-31TH with the 2089 bp BamHI/HindIII fragment of p31!N and the BamHI/KpnI-digested vector pUC19 resulted in pUC-31TH. This procedure fused the NcoI site created at the start codon via the HindIII site of the p31 promoter to avoid insertion at the aforementioned −103 site.

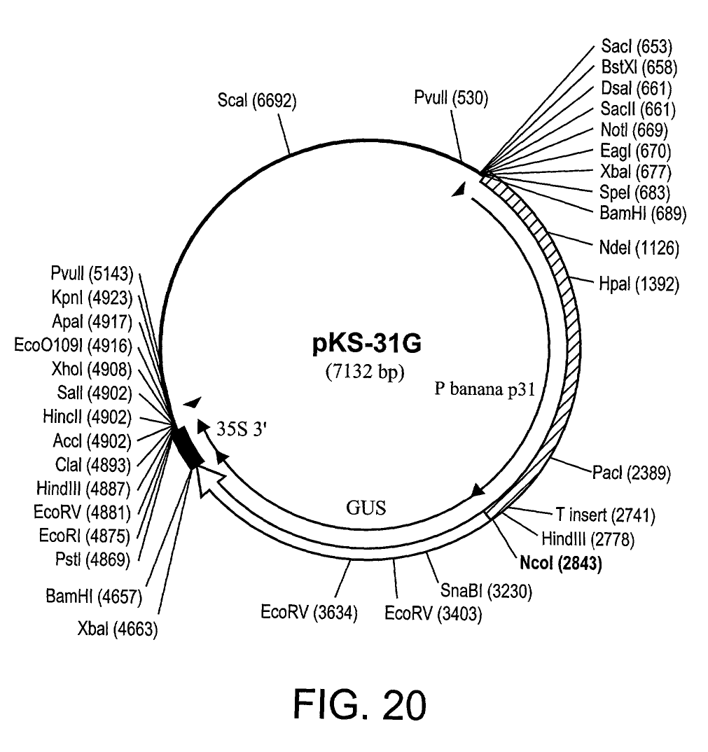

The p31 promoter was then fused with the reporter gene β-glucuronidase (GUS), the expression of which can be evaluated by histochemical staining in plant tissues (Jefferson, R. A. (1987), Plant Mol. Biol. Rep. 5:387-405; Jefferson et al. (1987), EMBO J. 13:3901-3907). The 2026 bp NcoI/PstI fragment of pRTL2-GUS (Carrington and Freed (1990) J. Virol. 64:1590-1597) containing the GUS coding sequence and the cauliflower mosaic virus (CaMV) 35S RNA 3′ end was ligated with the 2154 bp BamHI/NcoI fragment of pUC-31TH containing the banana p31 promoter and the BamHI/PstI-digested vector pBluescriptKS resulting in vector pKS-31G (FIG. 20).

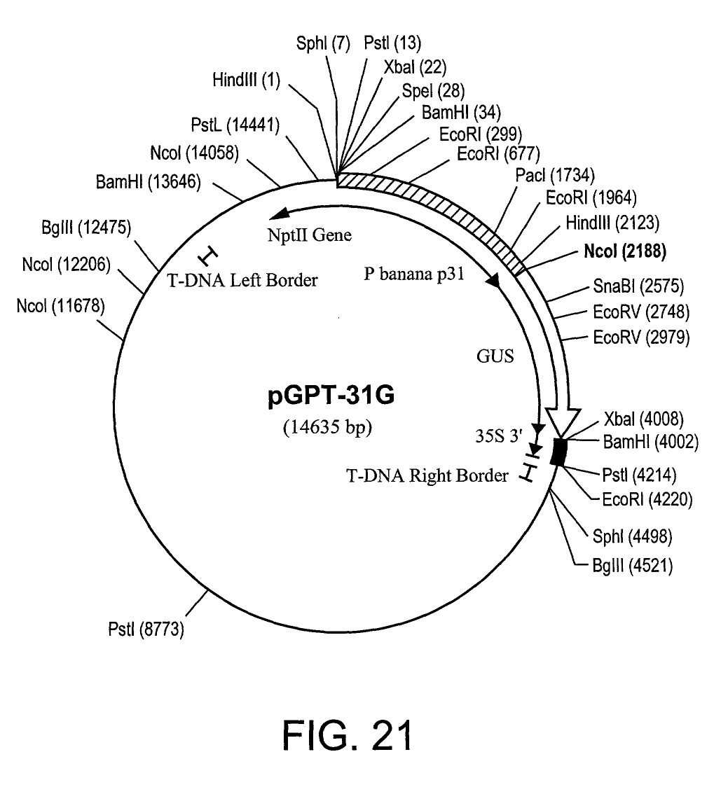

The p31-GUS expression cassette was inserted into a binary T-DNA vector for use in Agrobacterium-mediated plant transformation. The 2166 bp XbaI/NcoI fragment of pKS-31G containing the p31 promoter and the 2032 bp NcoI/EcoRI fragment of pKS-31G containing the GUS coding sequence and the CaMV 35S RNA 3′ end were ligated with XbaI/EcoRI-digested pGPTV-KAN (Becker et al. (1992) Plant Mol. Biol. 20:1195-1197) to construct pGPT-31G (FIG. 21). Recombinant clones were confirmed by four separate restriction digests with EcoRI, XbaI, XbaI/NcoI, or BamHI/HindII. Agrobacterium tumefaciens LBA4404 was transformed with a positive clone. Transformed Agrobacterium were identified by plasmid preparation and digestion with EcoRI.

Transformation of Tomato and Evaluation of GUS Expression

Tomato (Lycopersicon esculentum) variety TA234 was transformed with pGPT-31G by Agrobacterium-mediated transfer of the T-DNA and regeneration of whole plants on medium containing kanamycin. Transgenic lines were evaluated by Northern blot for expression of mRNA encoding NptII, and several lines were selected for transplant to soil and growth in the greenhouse. Fruits of mature transgenic and control plants were assayed for GUS activity by the histochemical staining method (Jefferson, R. A. (1987), Plant Mol. Biol. Rep. 5:387-405; Jefferson et al. (1987), EMBO J. 13:3901-3907).

### Results

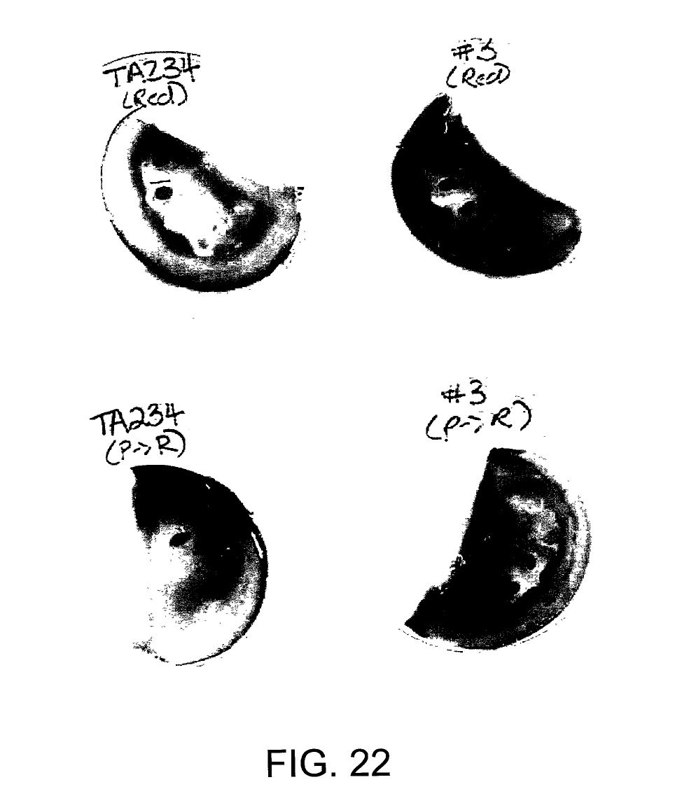

FIG. 22 indicates that the expression of GUS is predominantly in the vascular and placental tissues in transgenic fruit, while no staining is observed in nontransgenic fruit. Although other additional tissues were subjected to staining, no GUS activity was observed in leaf, petiole, or stem tissues (data not shown). Thus, these data demonstrate that the p31 promoter derived from banana can drive fruit-specific heterologous gene expression in tomato. Further, these data provide support that monocot-derived (e.g. banana) promoters can be used to drive gene expression in dicots (e.g. tomato).

While the invention has been described and illustrated herein by references to various specific material, procedures and examples, it is understood that the invention is not restricted to the particular material, combinations of material, and procedures selected for that purpose. Numerous variations of such details can be implied and will be appreciated by those skilled in the art.

What is claimed is:

1. An isolated and purified banana DNA molecule, said DNA molecule being differentially expressed during banana fruit development.

2. A DNA molecule according to claim 1, wherein said DNA molecule encodes a protein selected from the group consisting of a starch synthase, a granule-bound starch synthase, a chitinase, an endochintinase, a beta-1,3 glucanase, a thaumatin-like protein, an ascorbate peroxidase, a metallothionein, a lectin, and another senescence-related protein.

3. A DNA molecule according to claim 1, selected from the group consisting of clones pBAN 3-33, pBAN 3-18, pBAN 3-30, pBAN 3-24, pBAN 1-3, pBAN 3-28, pBAN 3-25, pBAN 3-6, pBAN 3-23, pBAN 3-32, and pBAN 3-46.

4. A DNA molecule according to claim 1, wherein said DNA molecule has the nucleotide sequence selected from the group consisisting of SEQ ID NO: 1, SEQ ID NO: 2, and SEQ ID NO: 3.

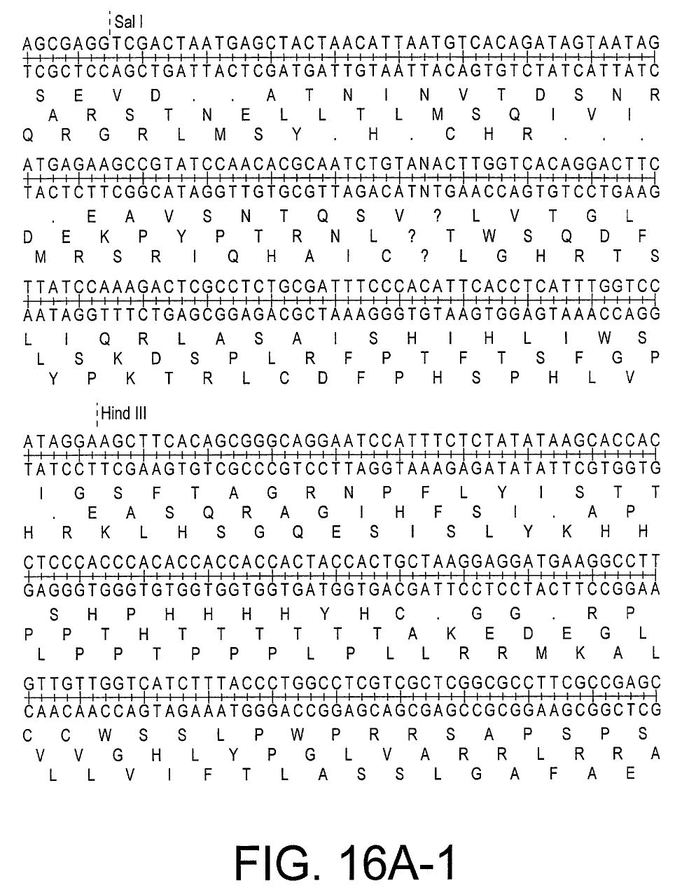

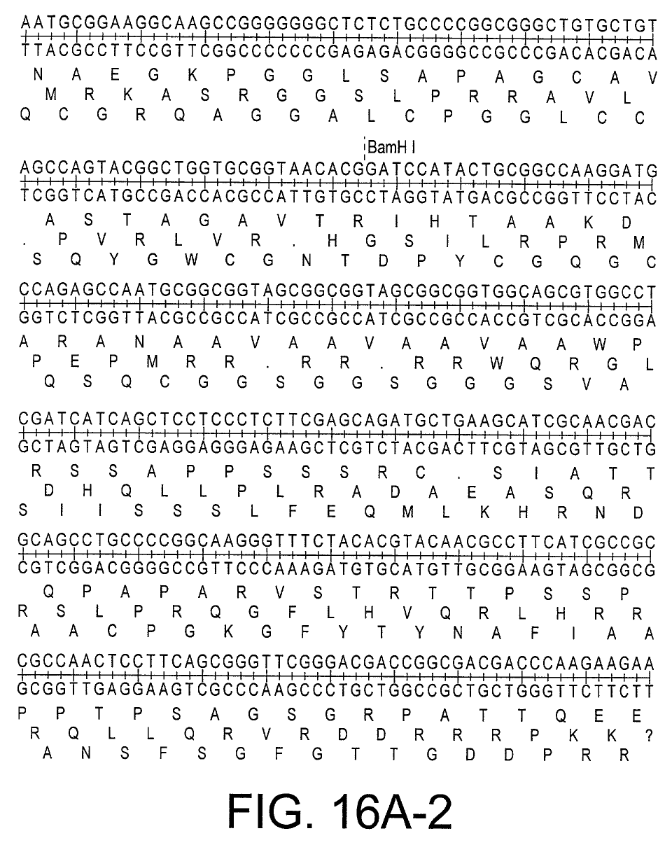

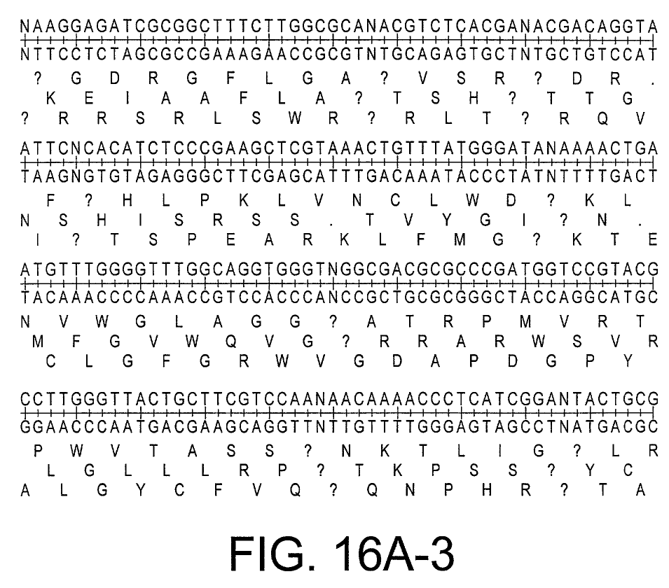

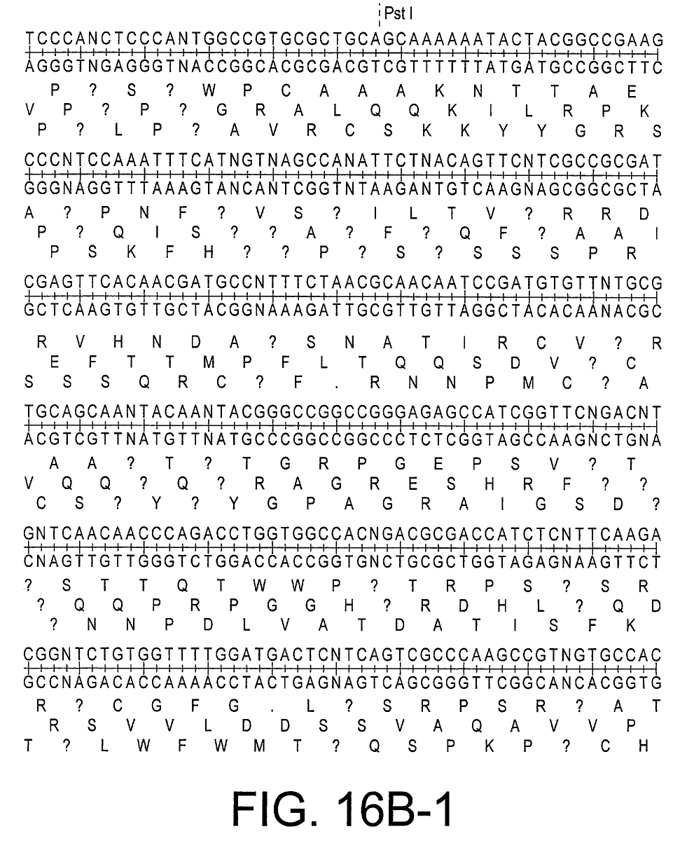

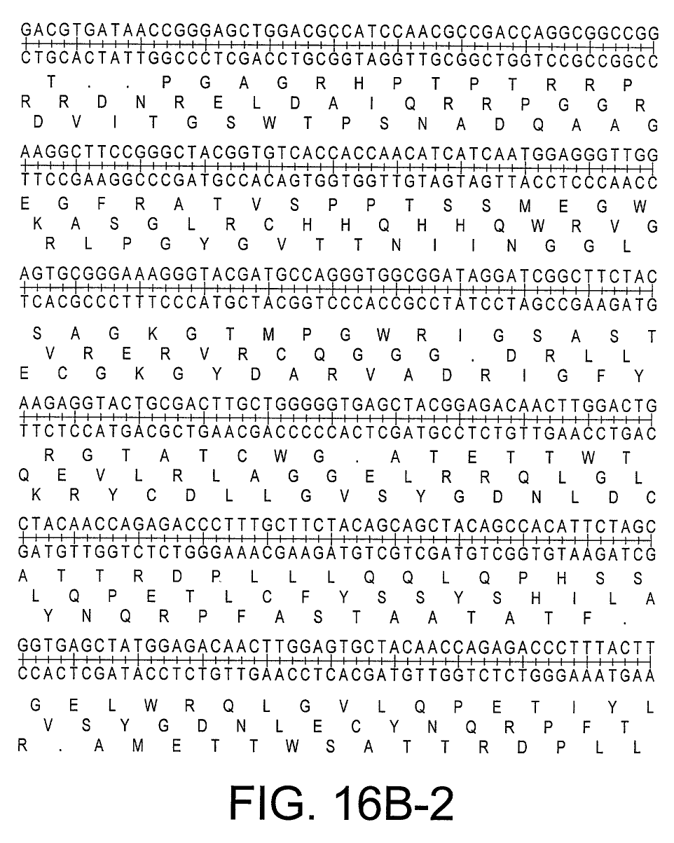

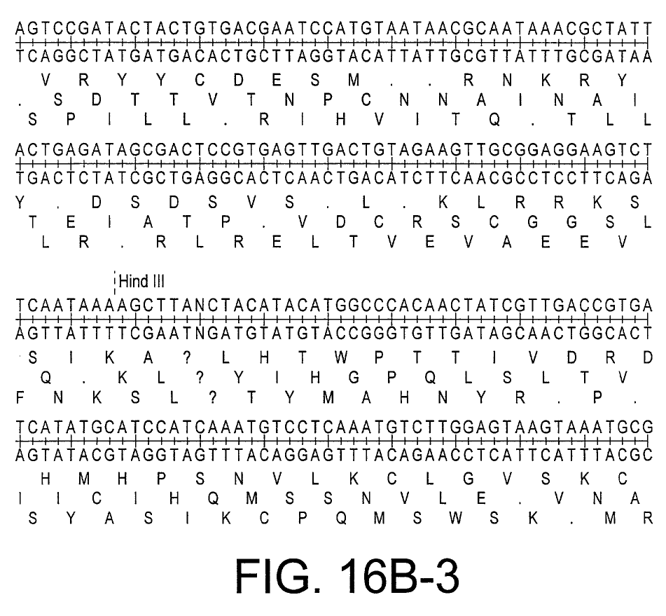

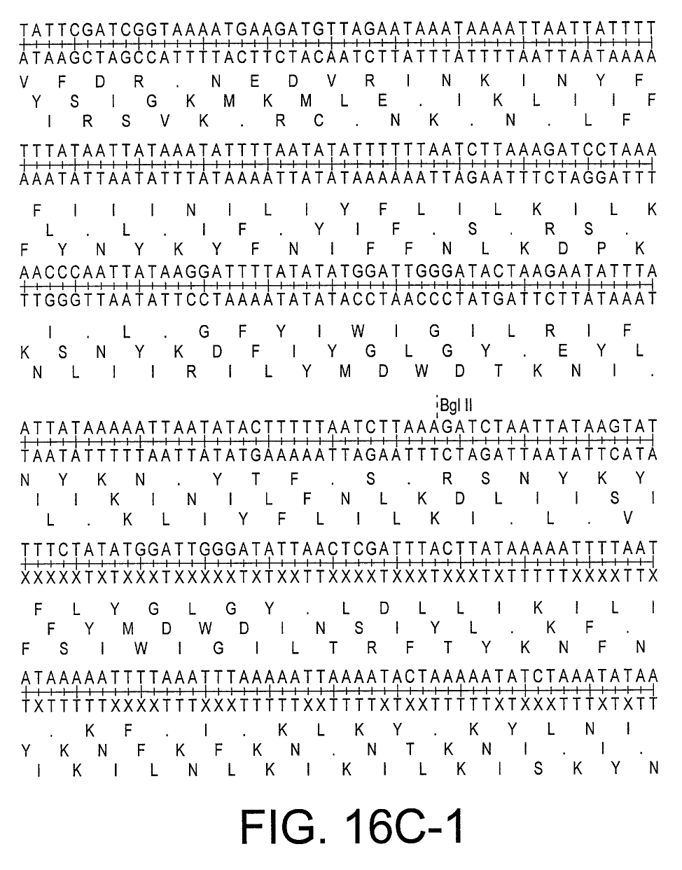

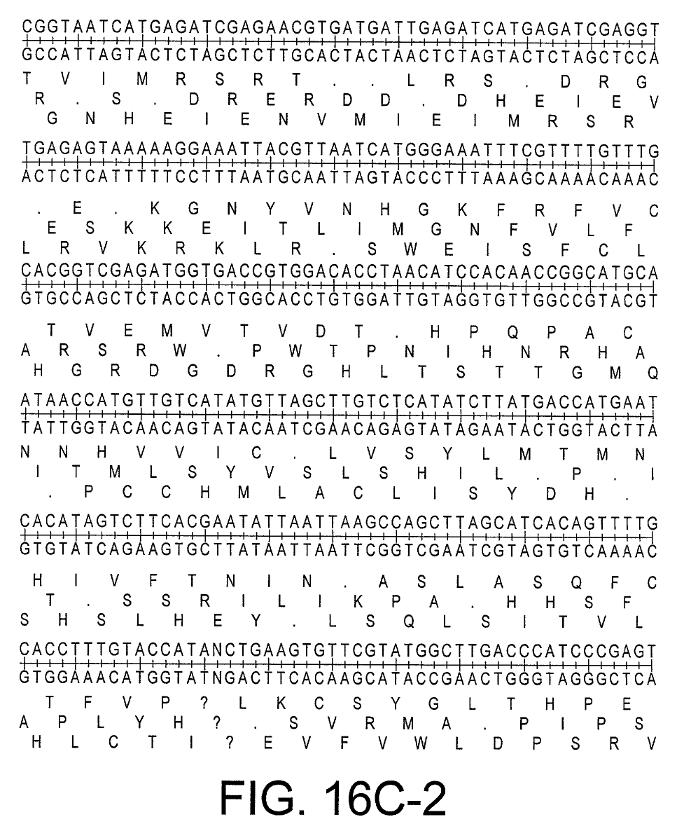

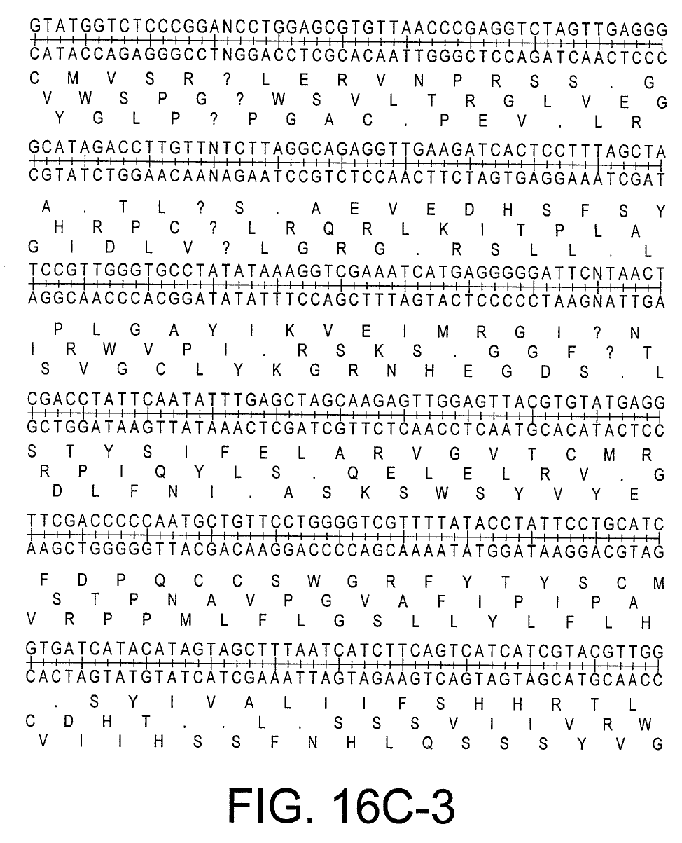

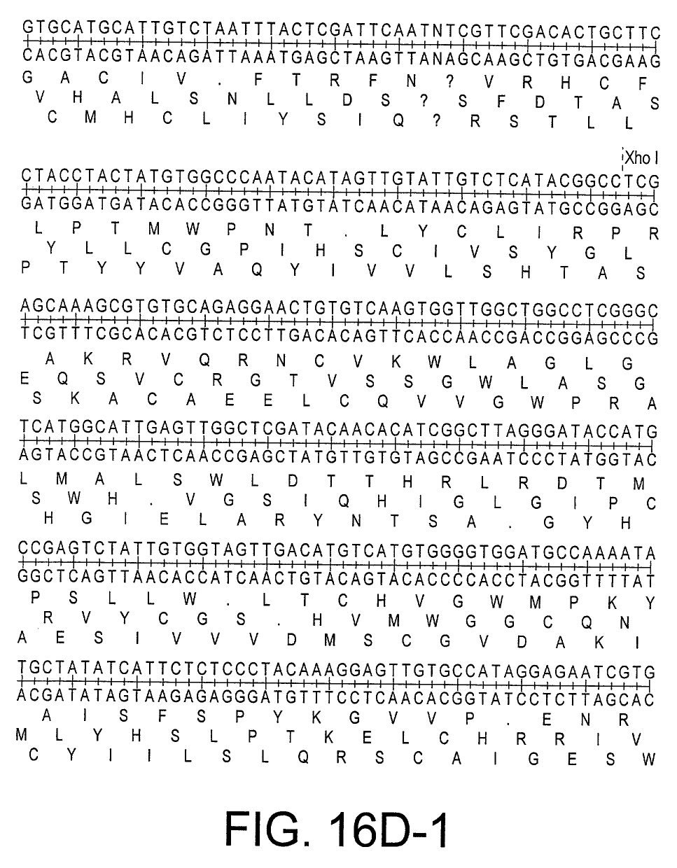

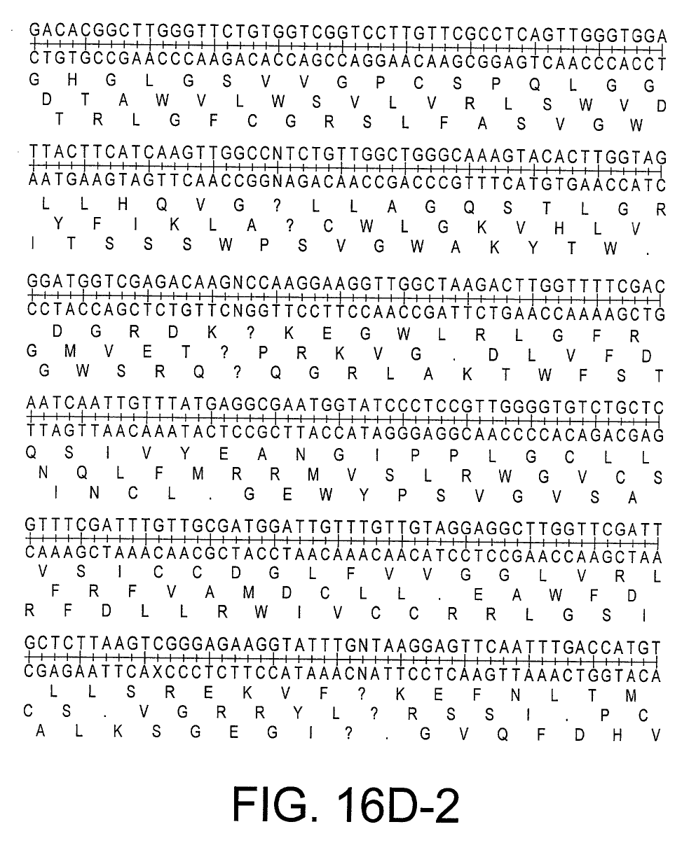

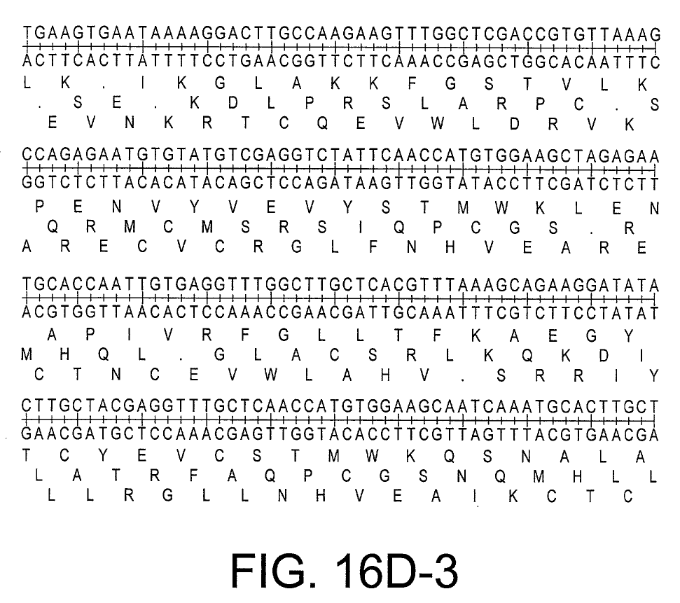

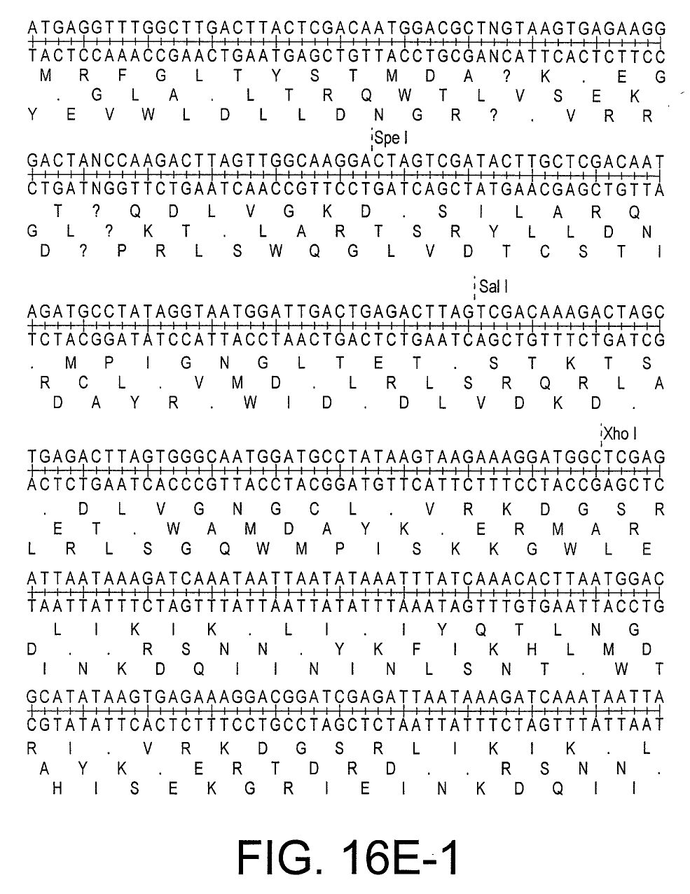

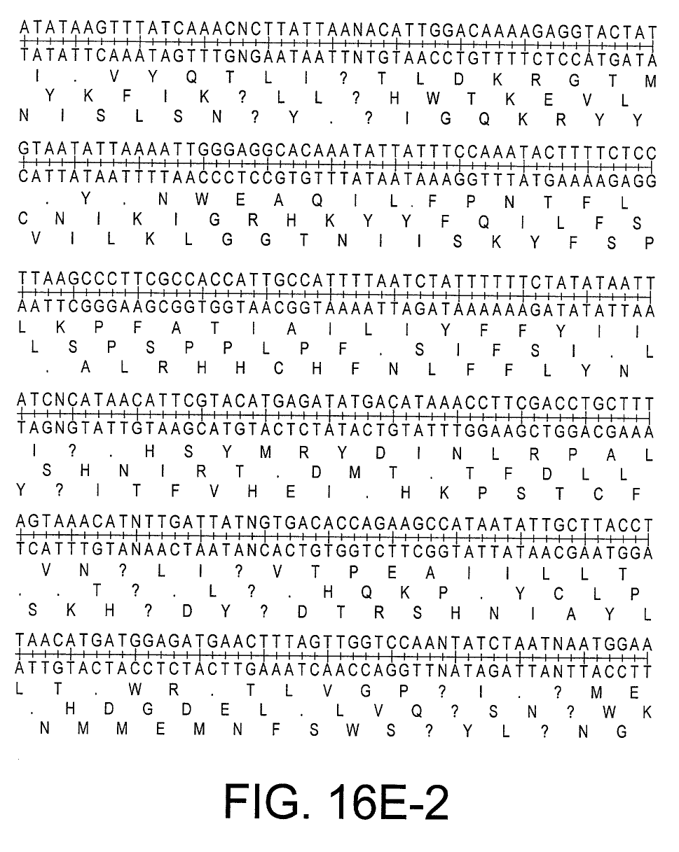

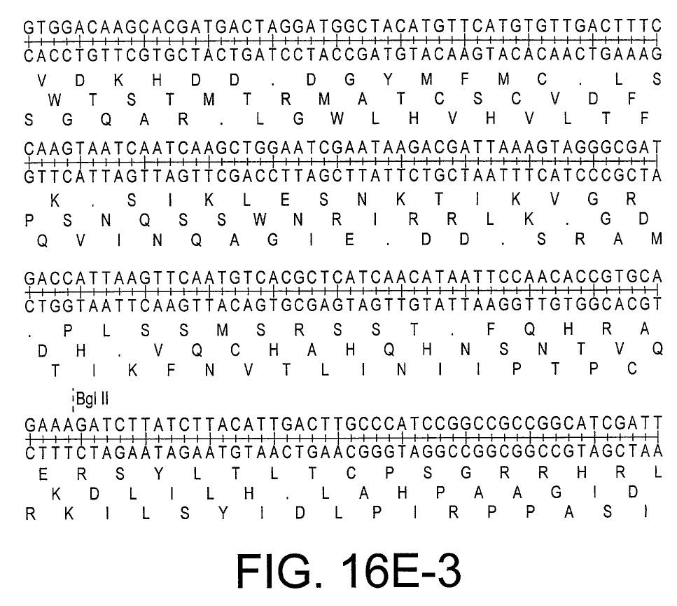

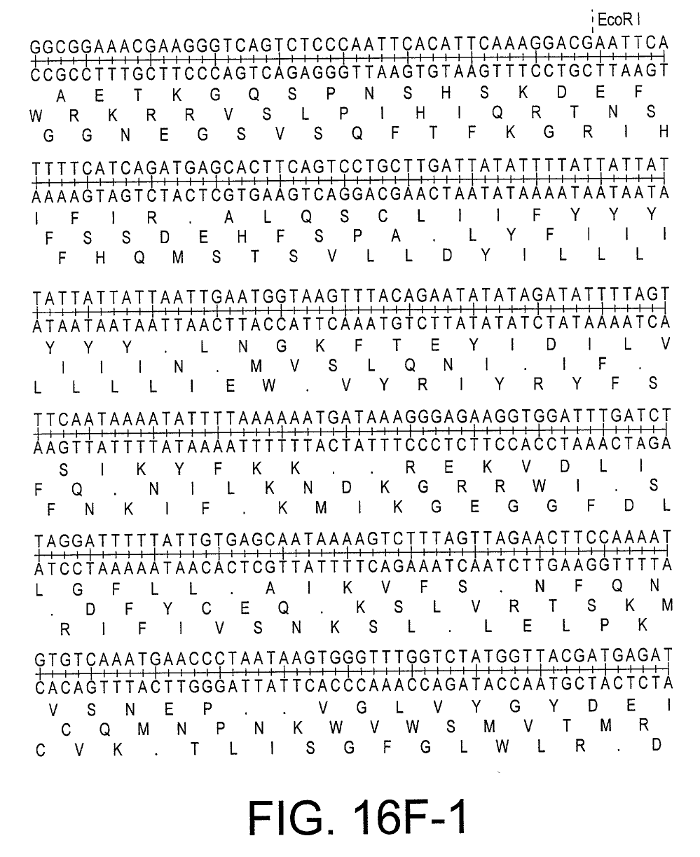

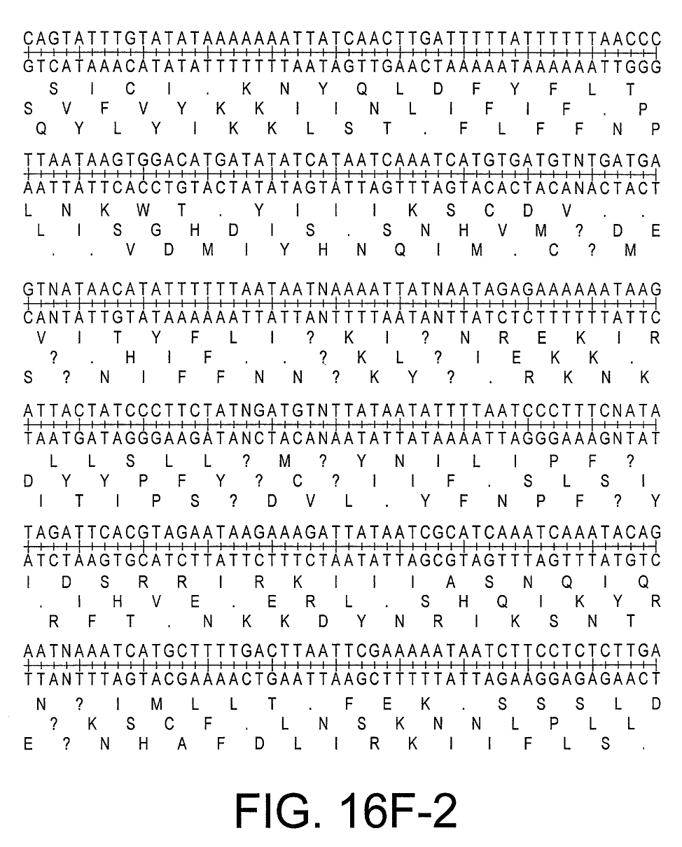

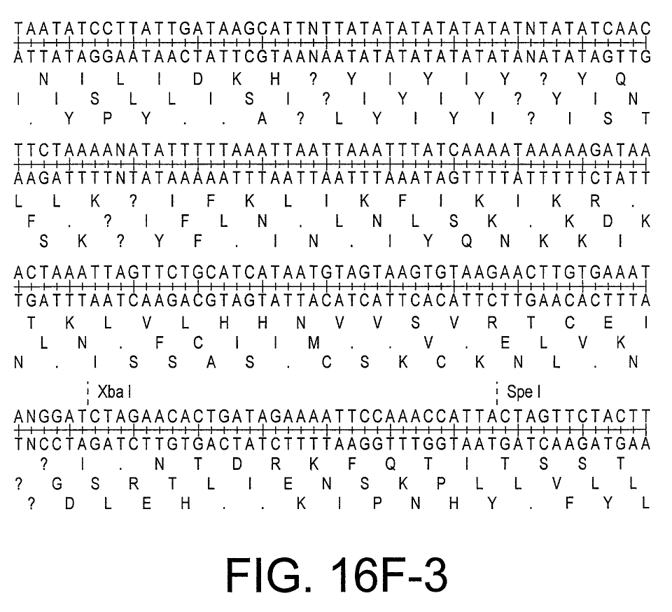

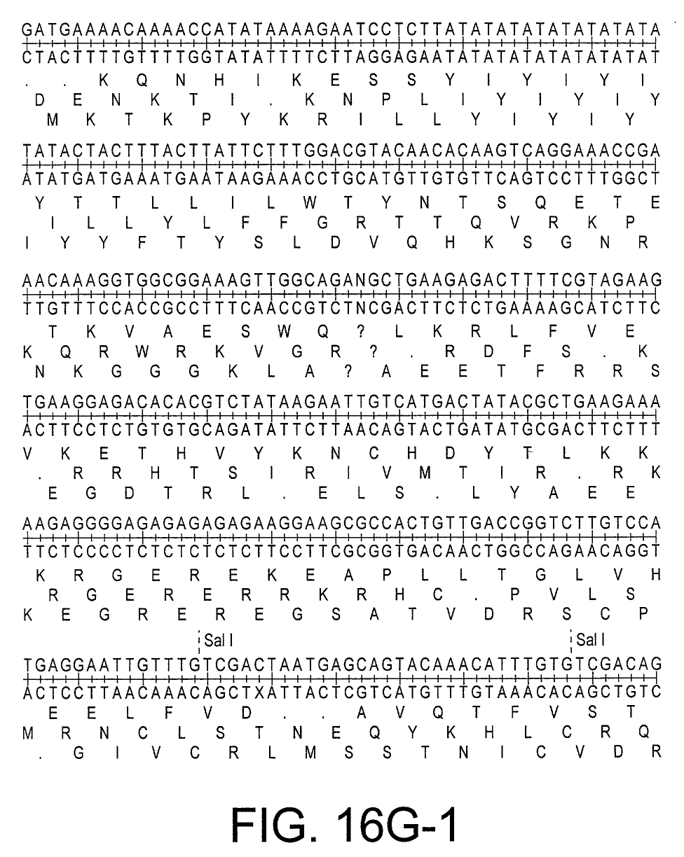

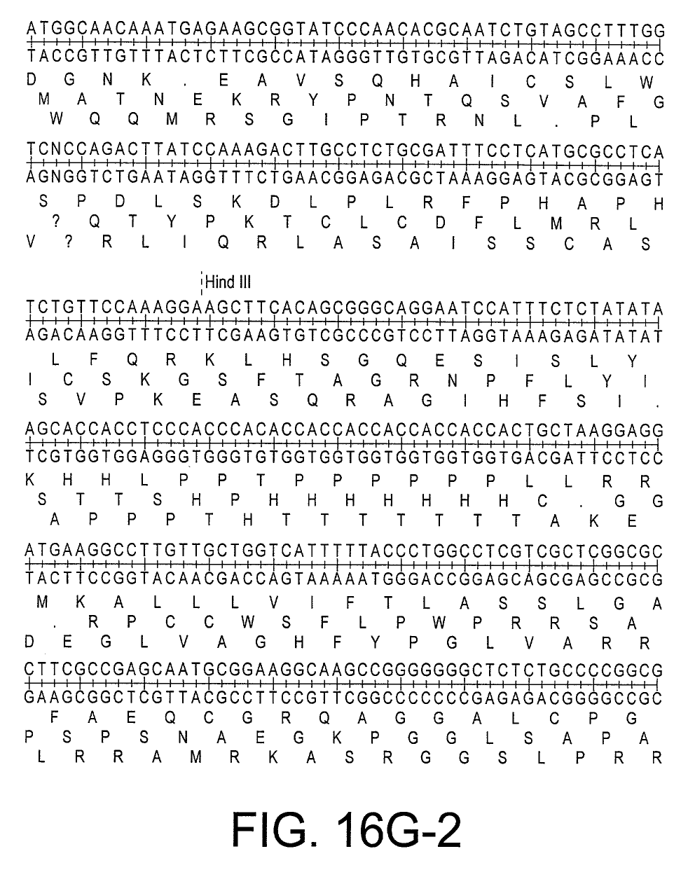

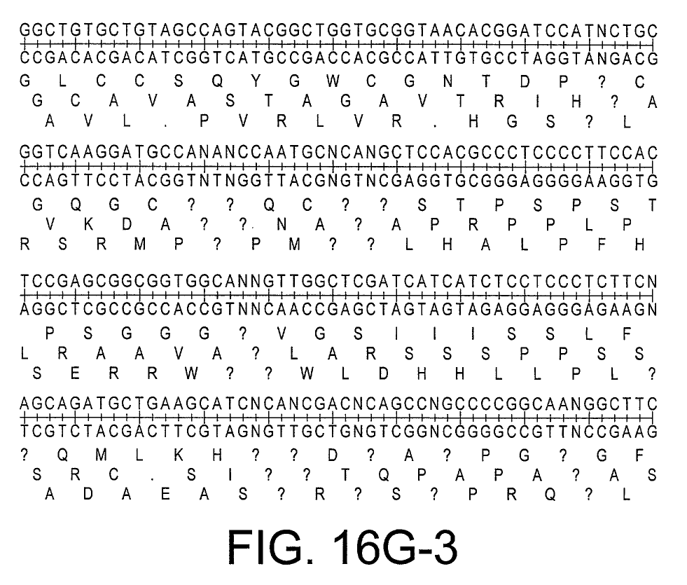

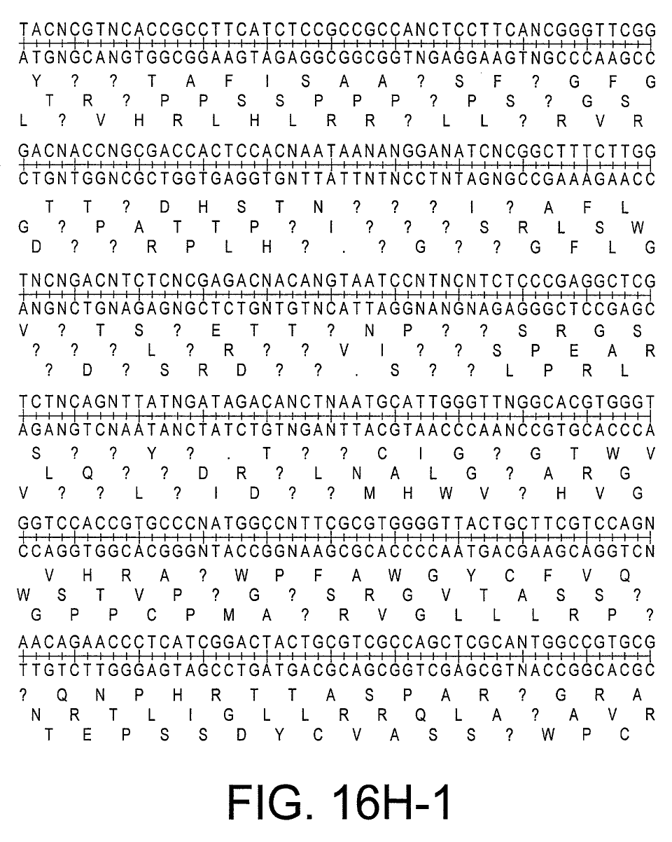

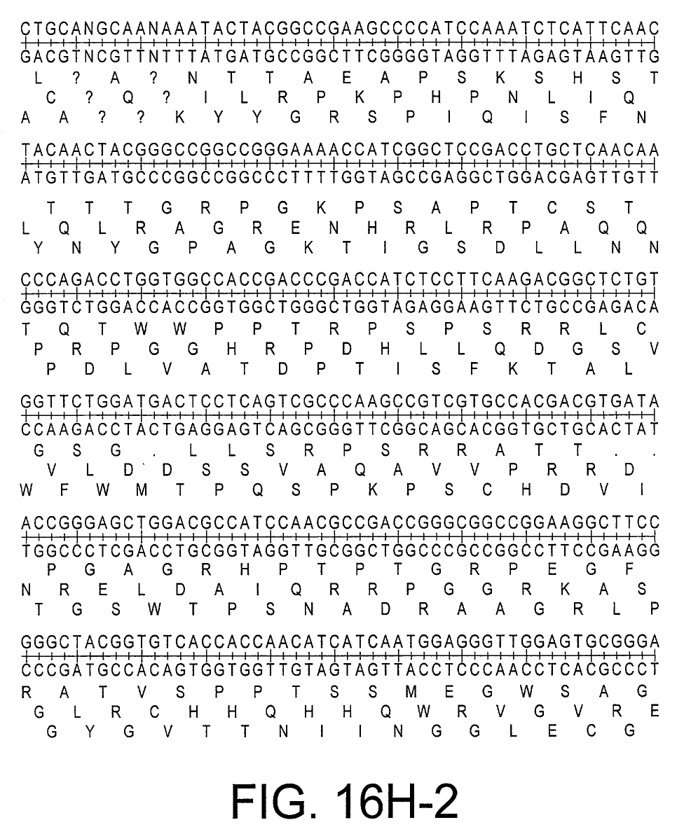

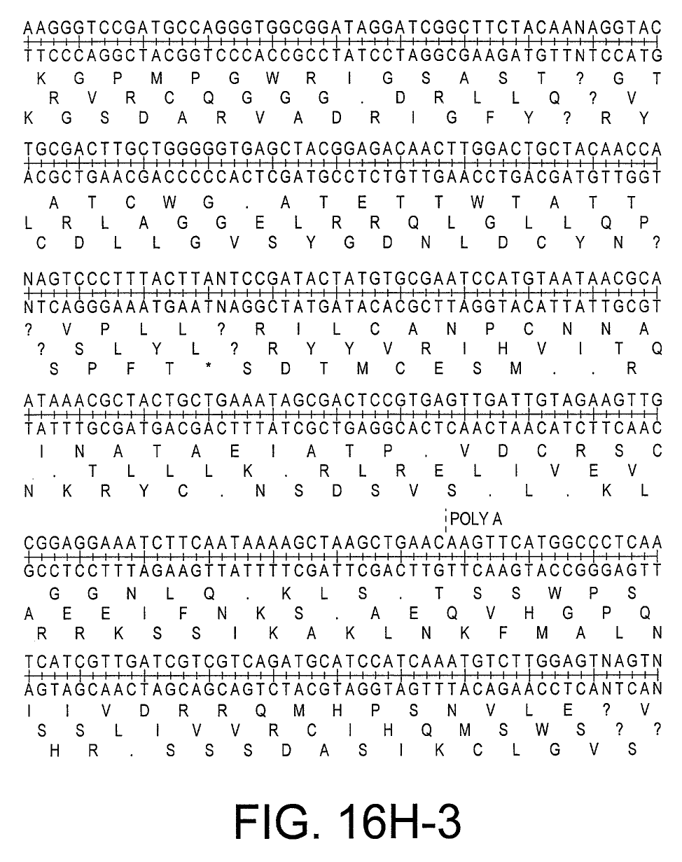

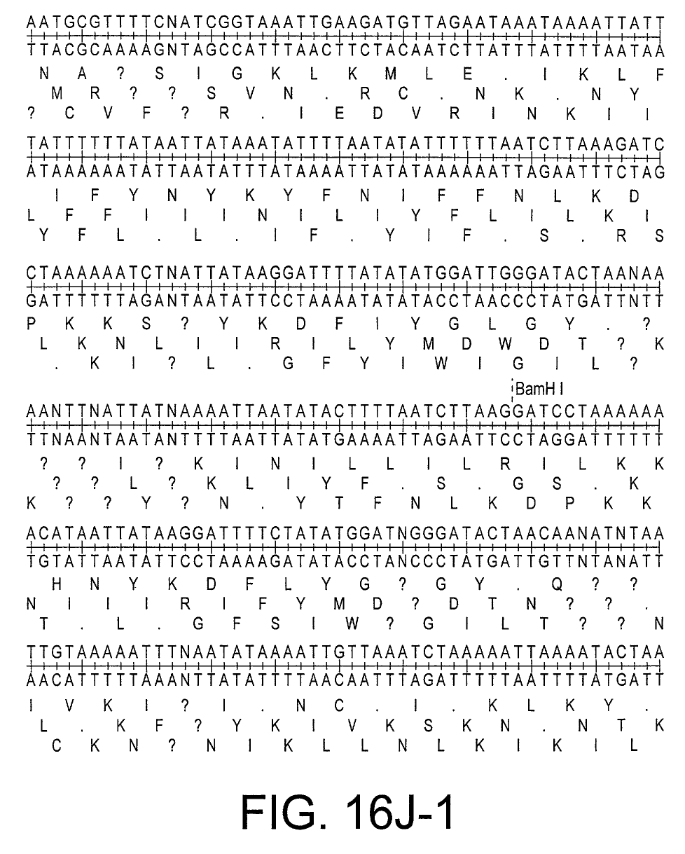

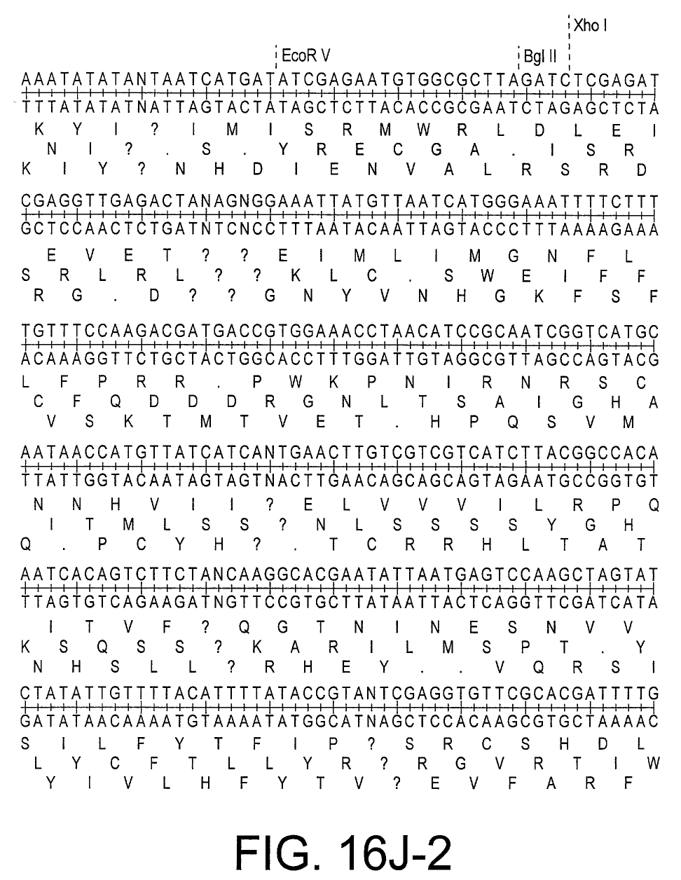

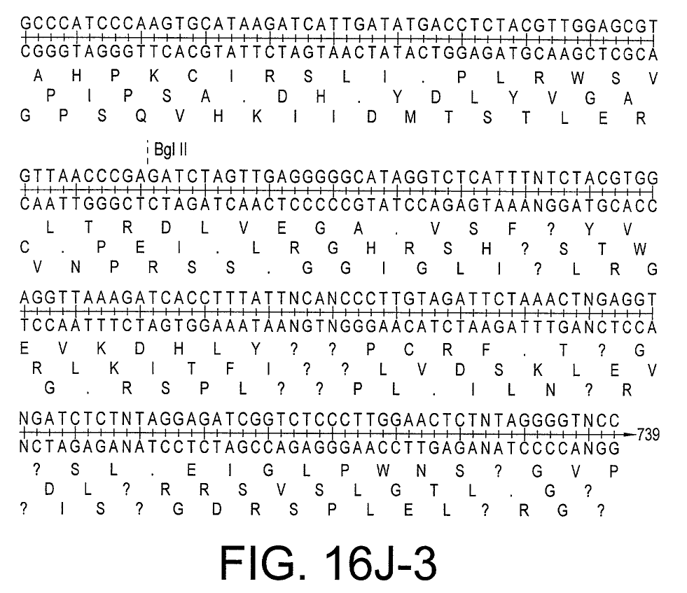

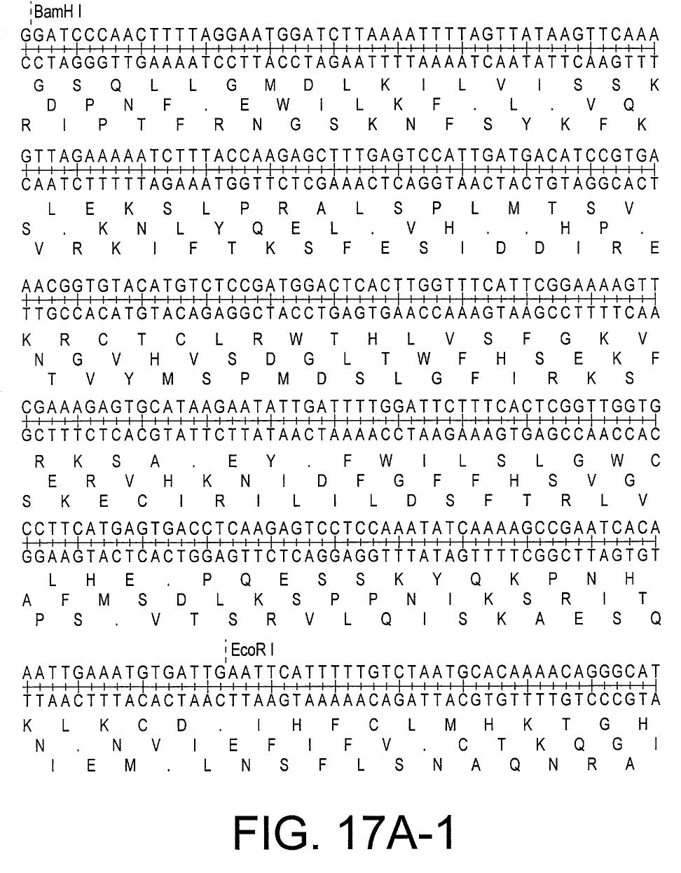

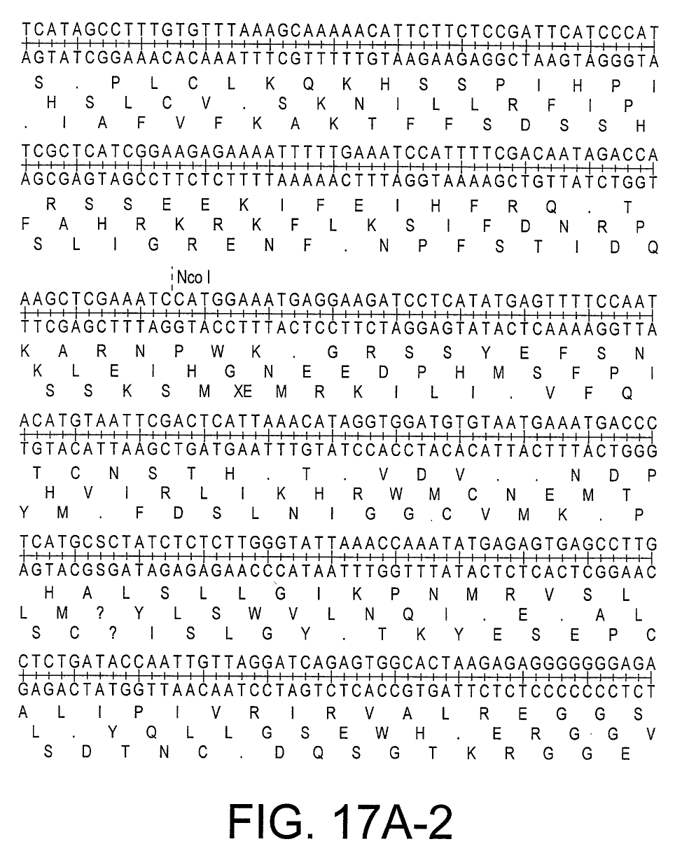

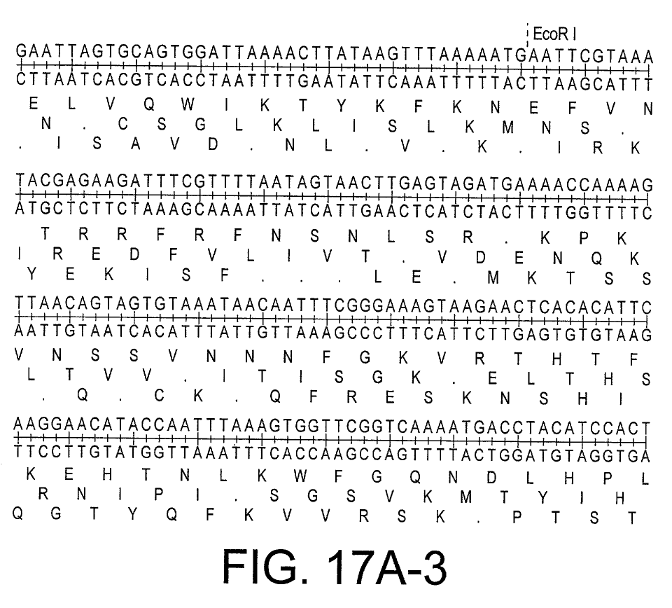

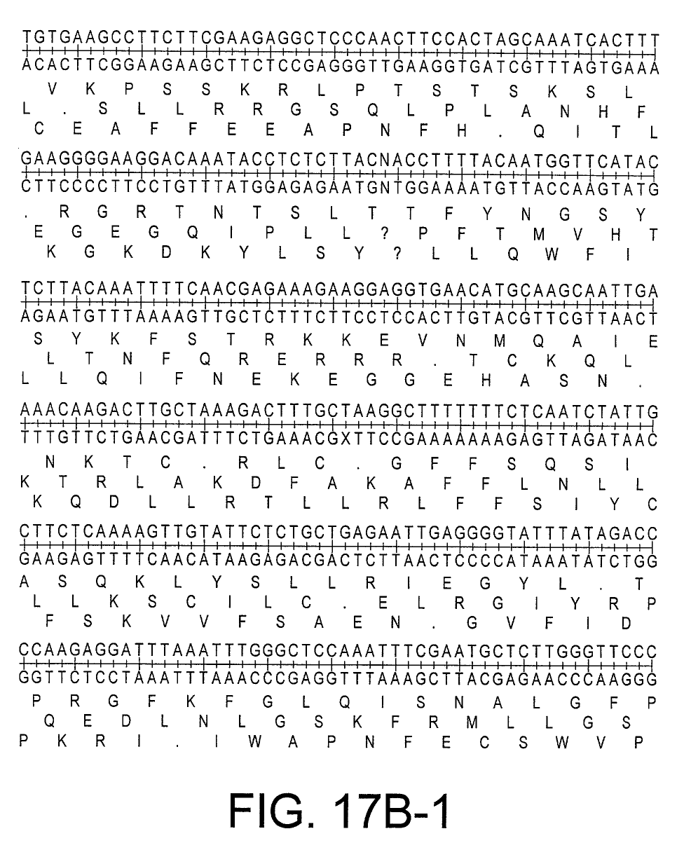

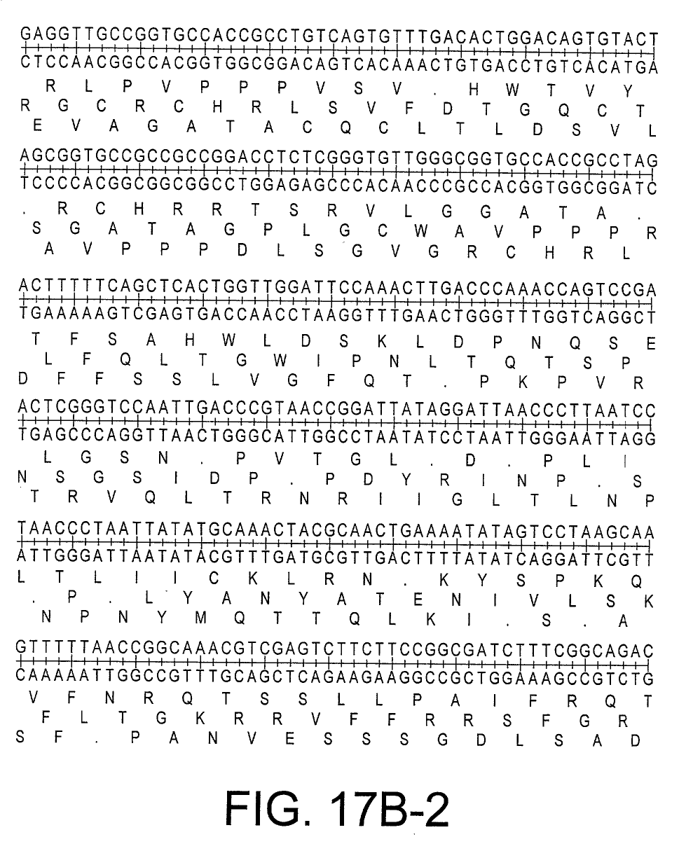

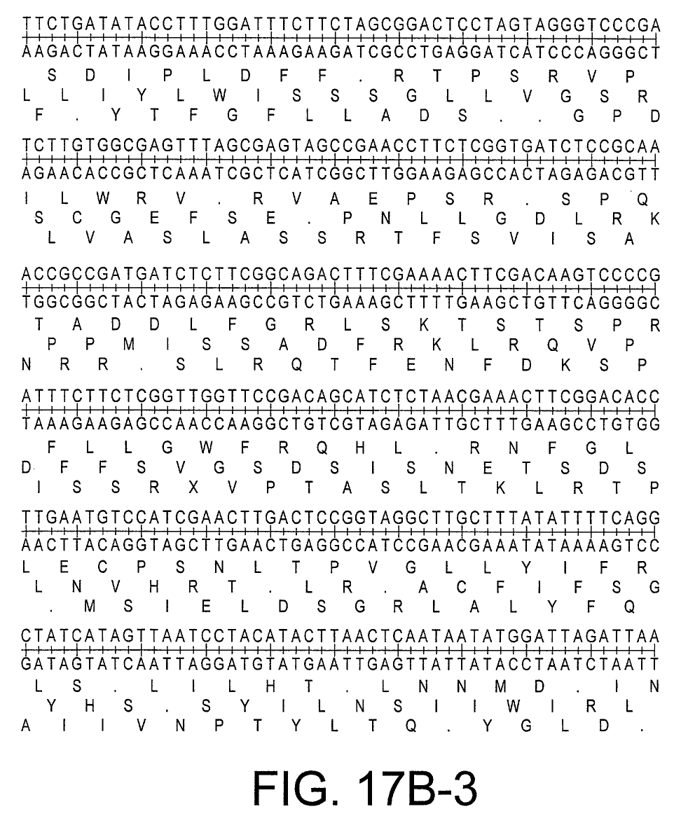

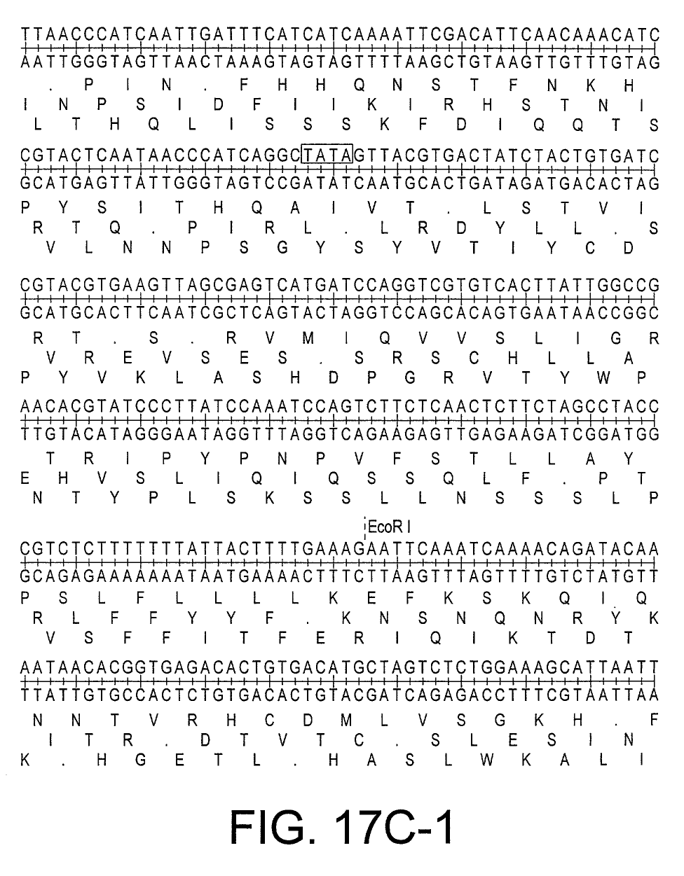

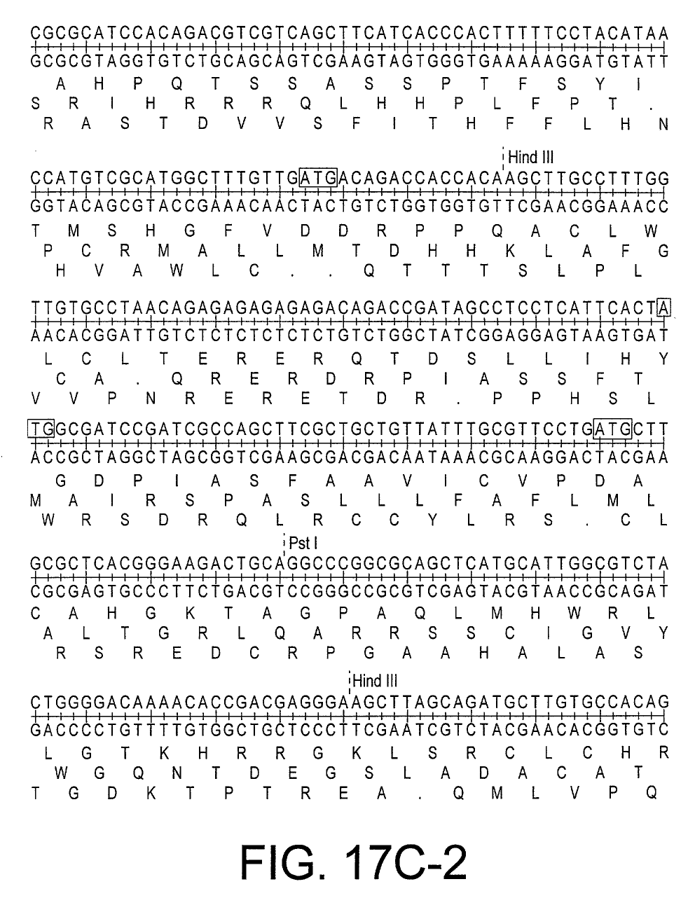

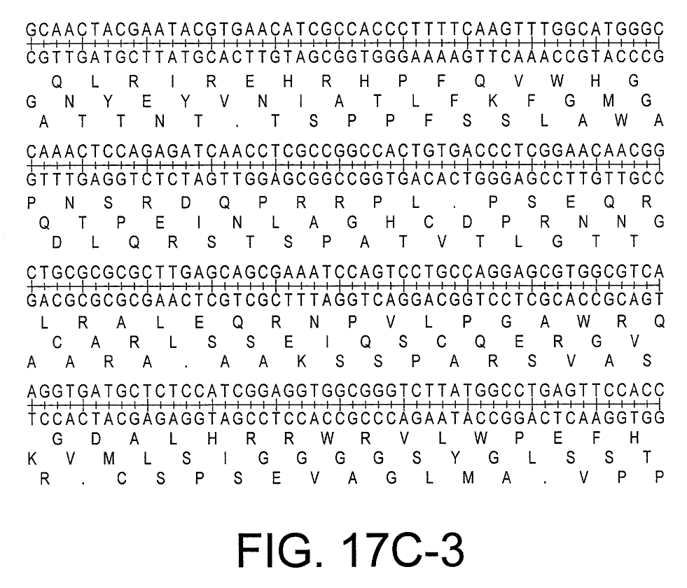

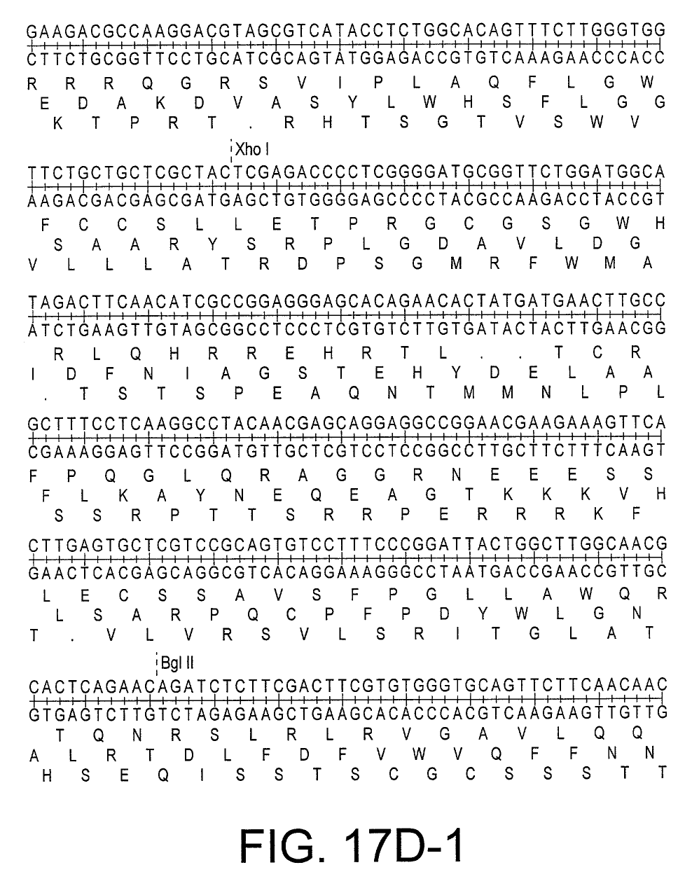

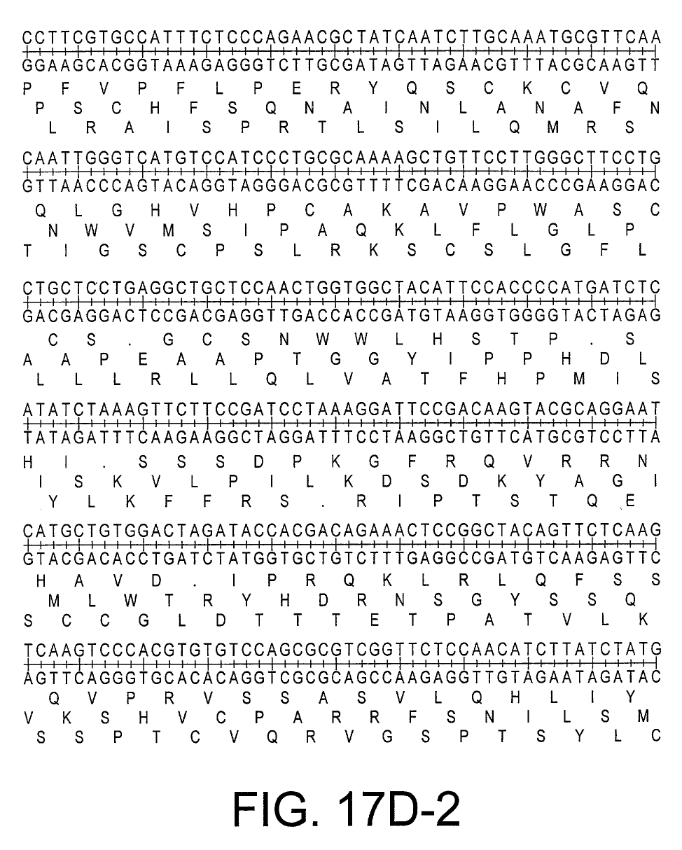

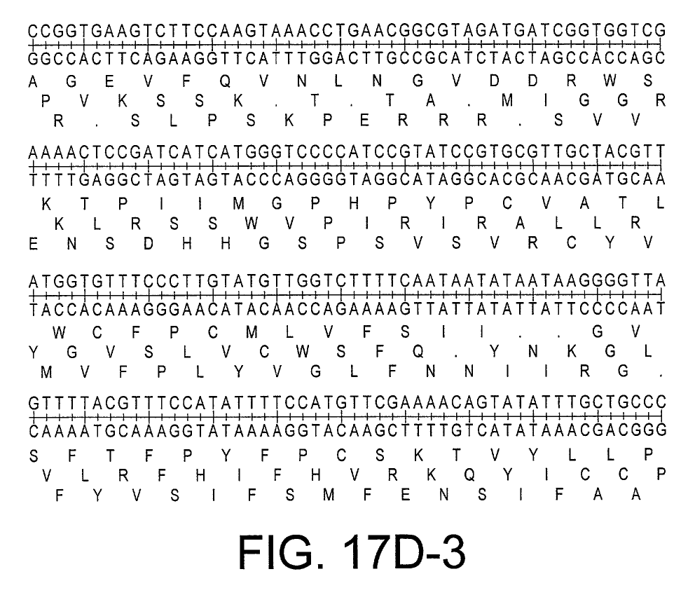

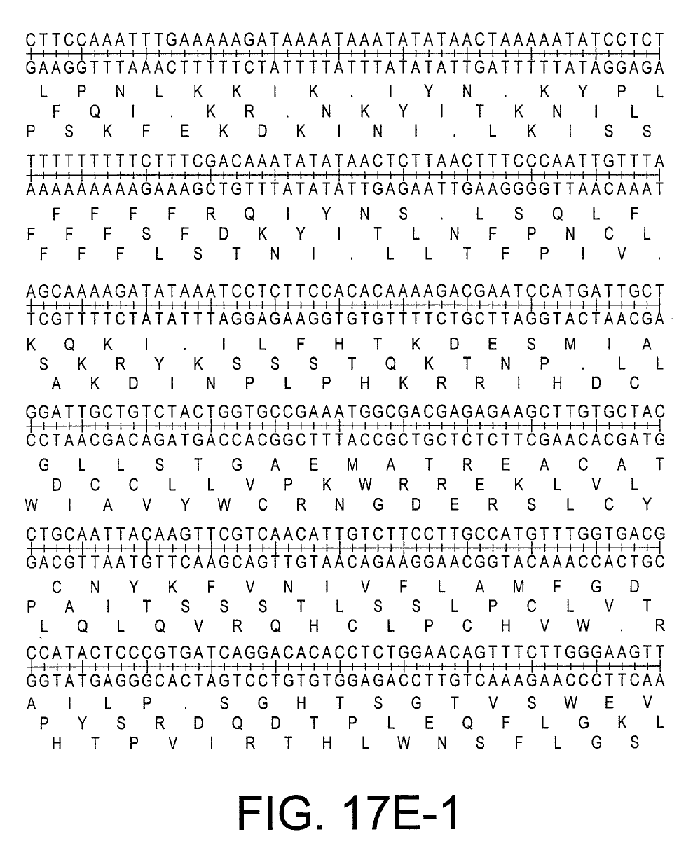

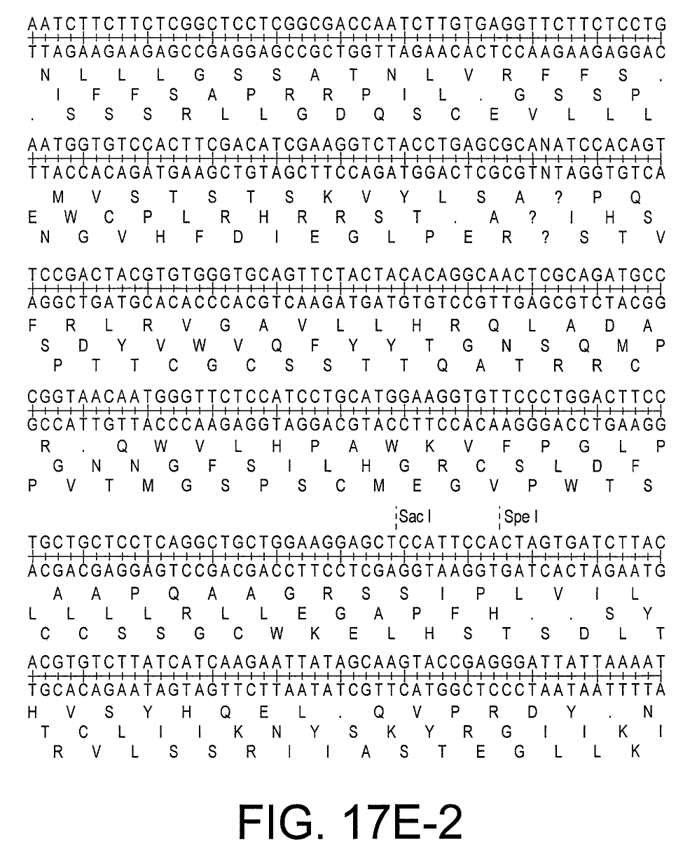

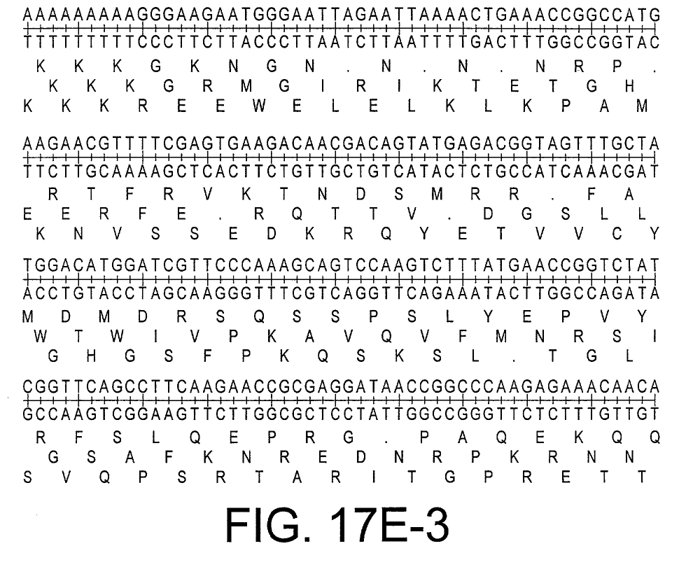

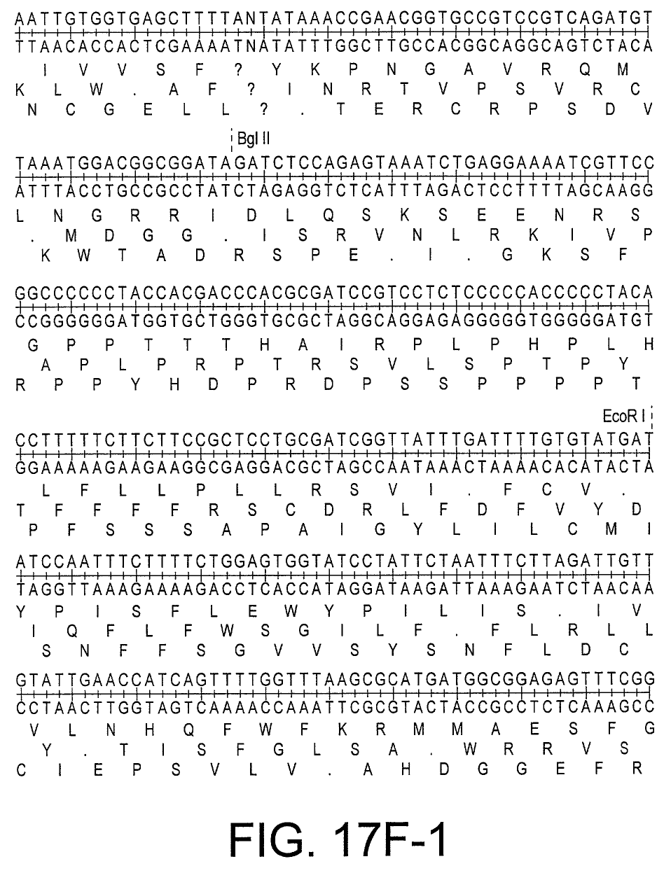

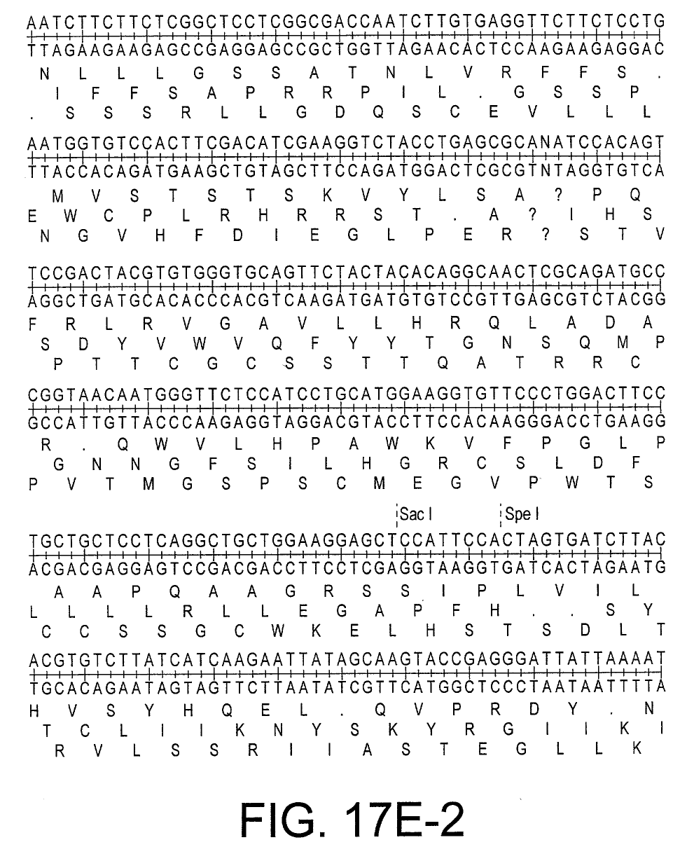

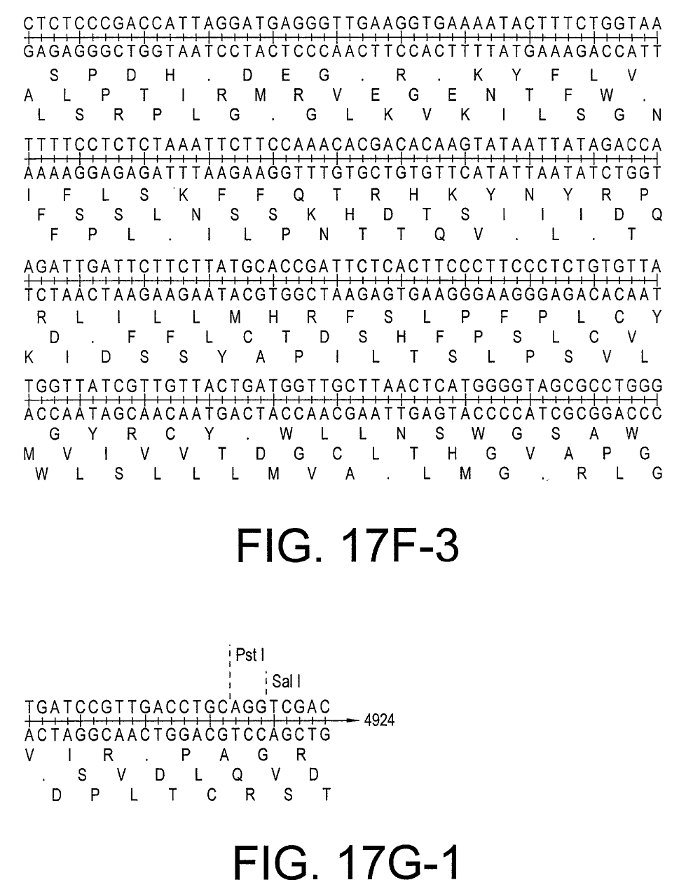

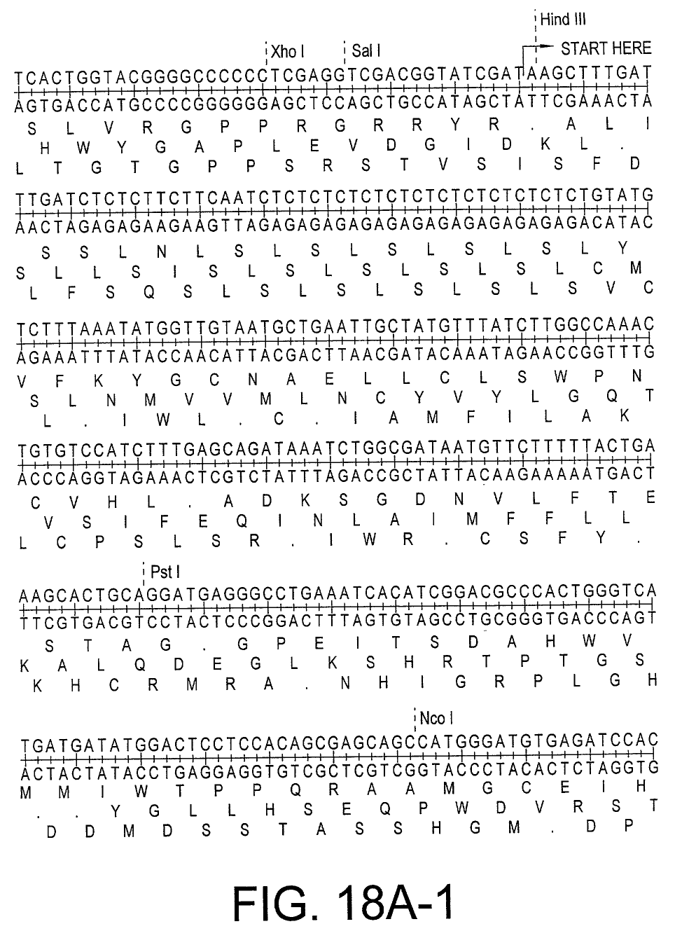

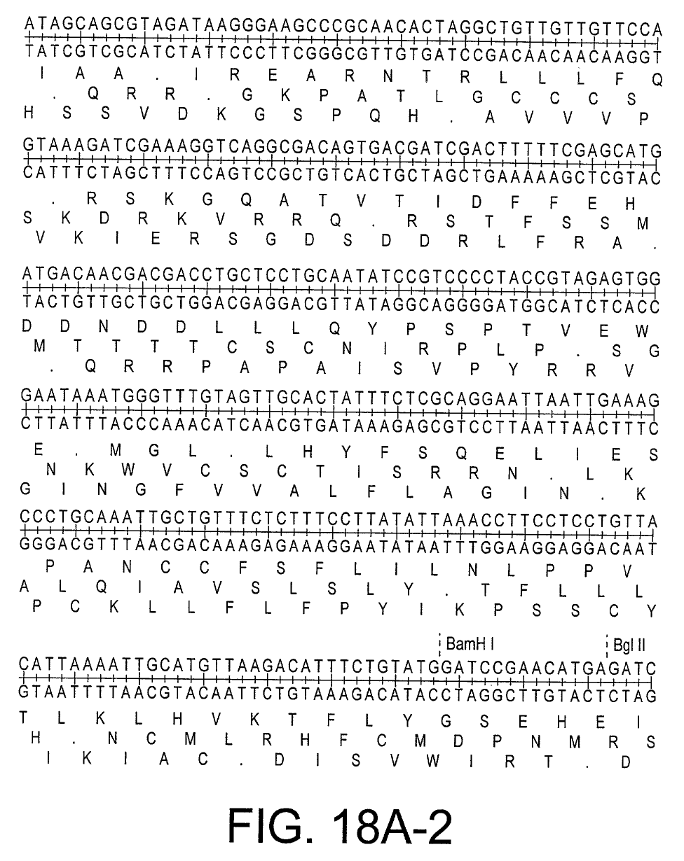

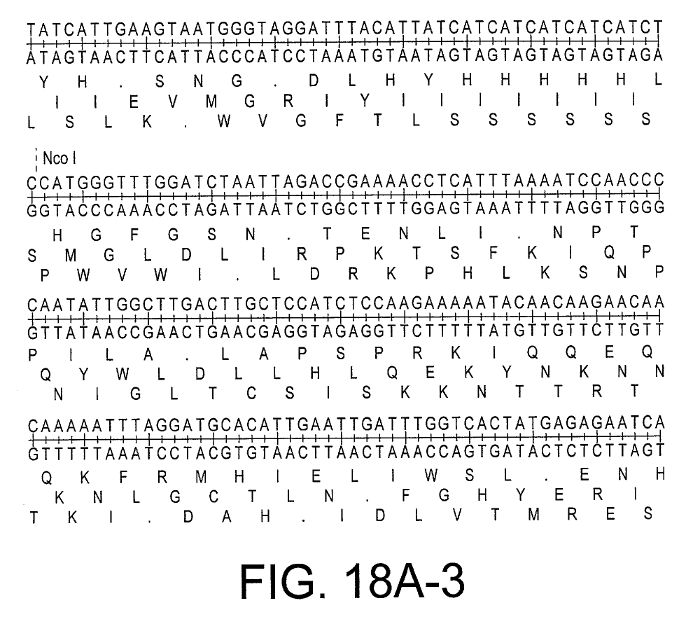

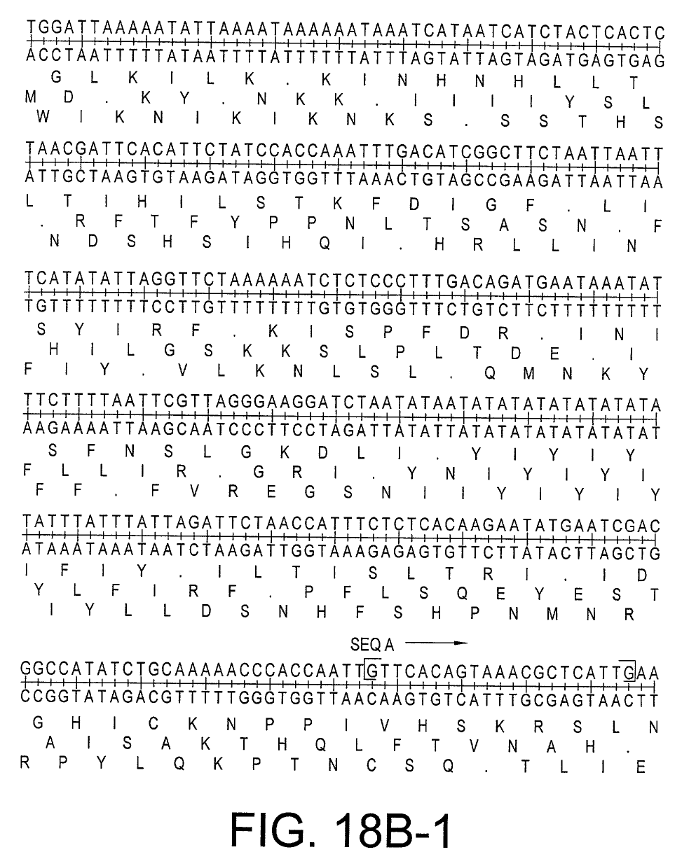

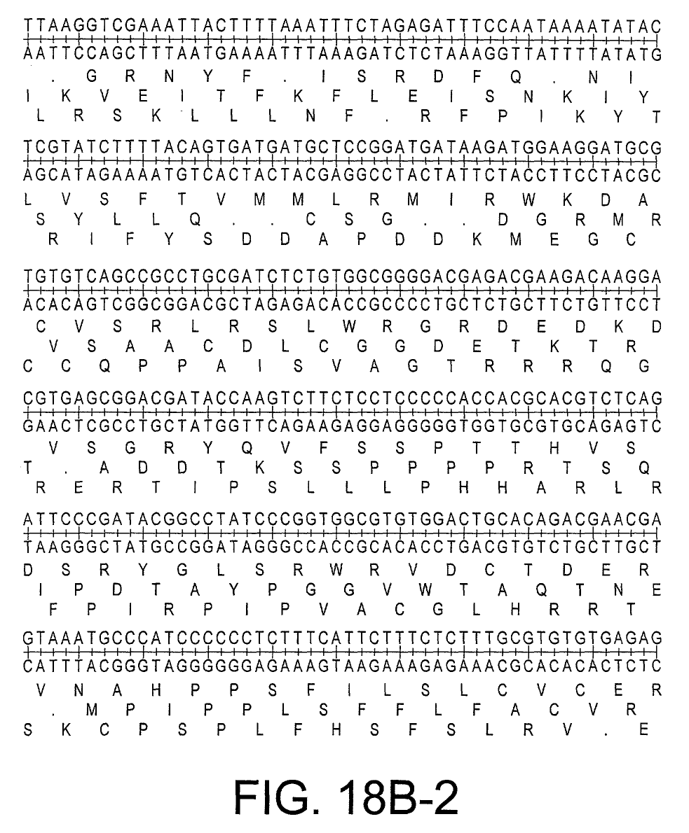

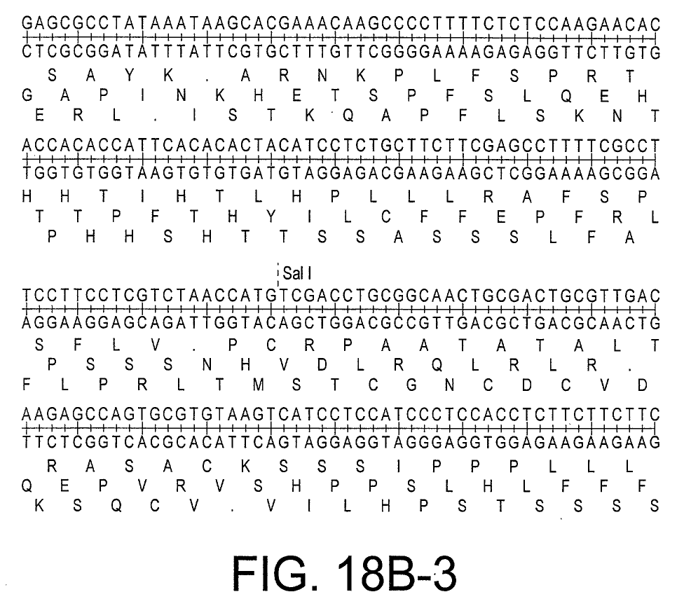

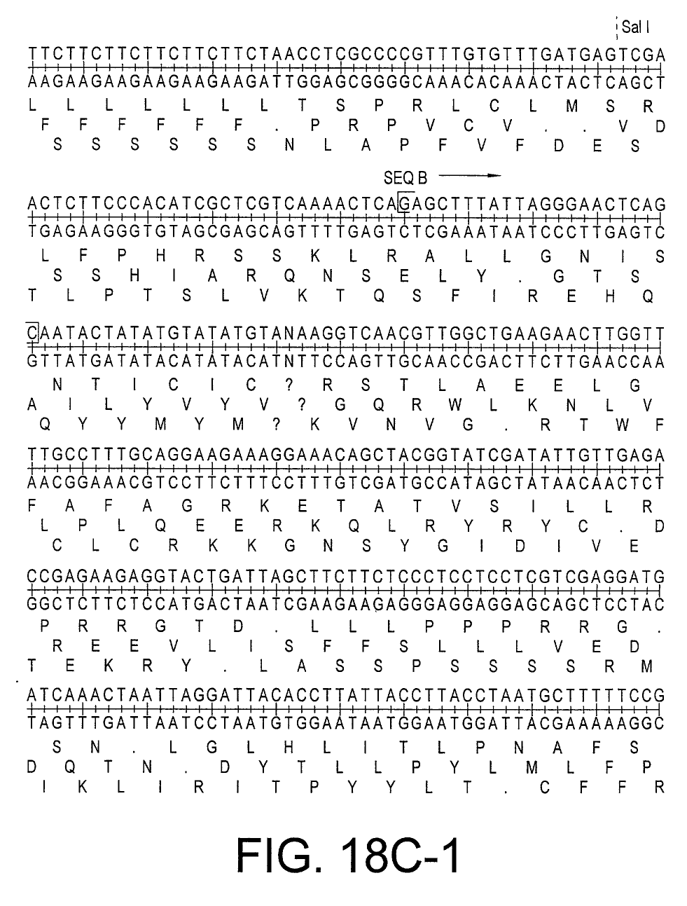

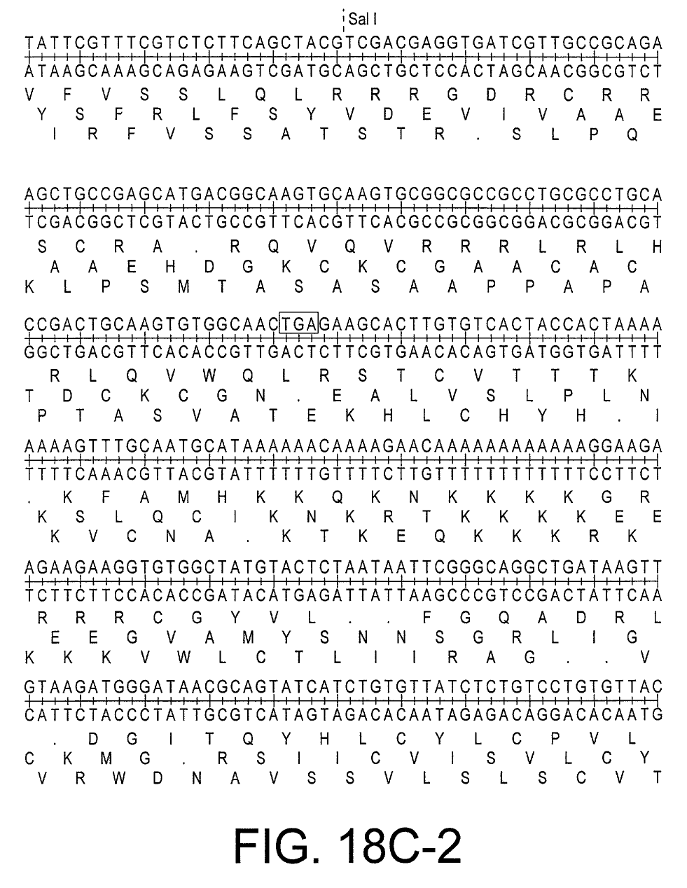

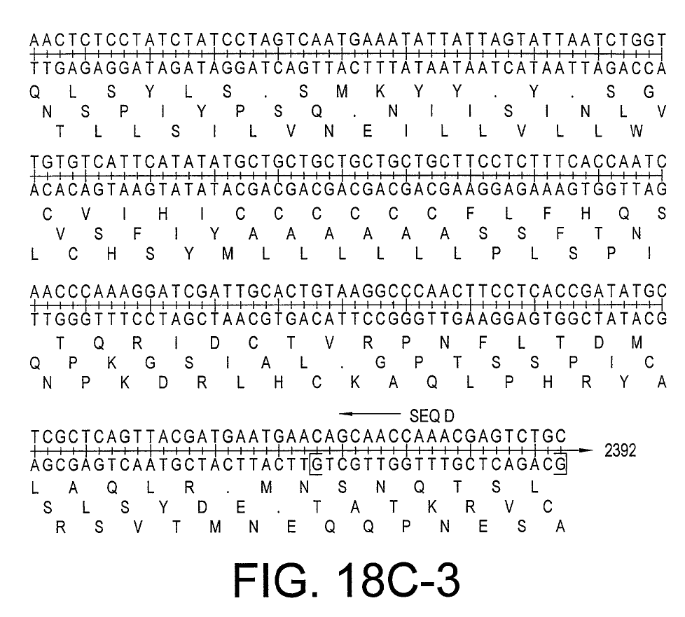

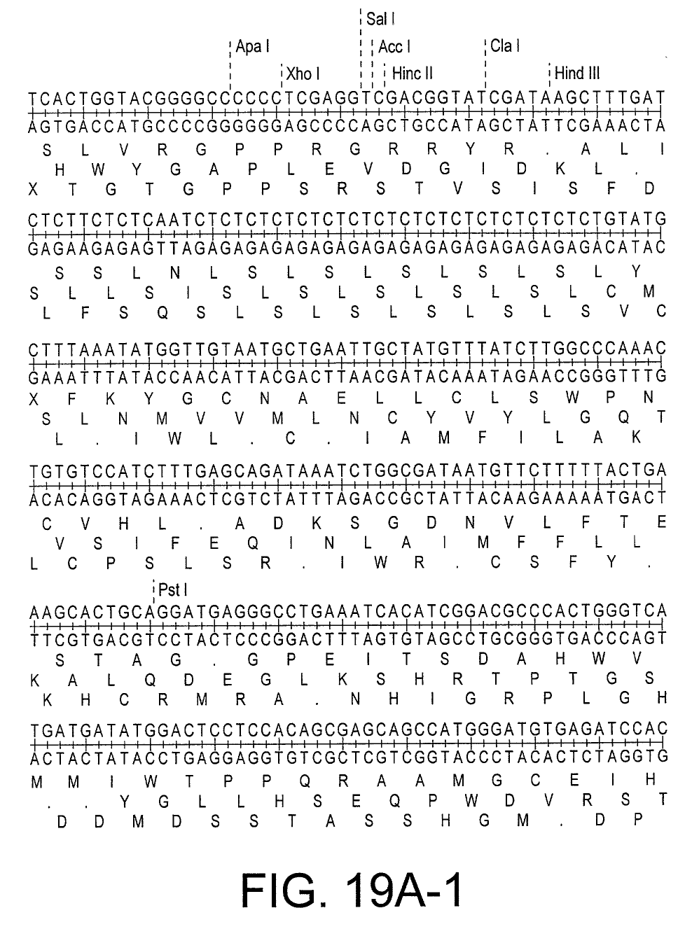

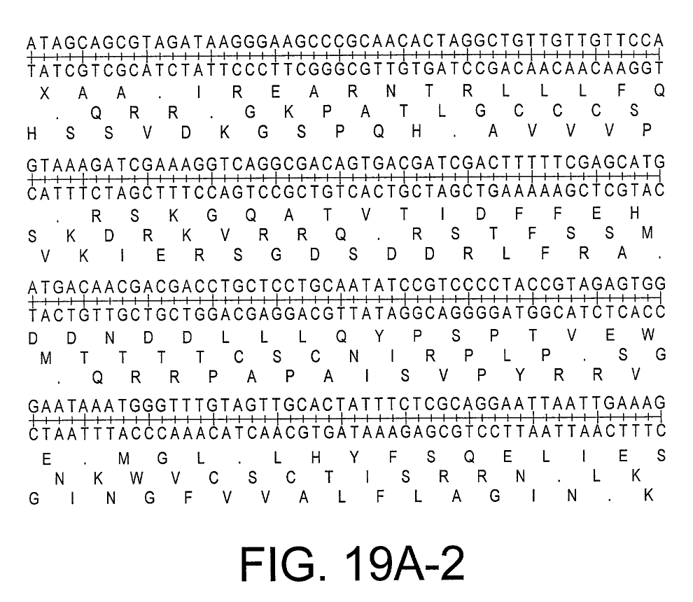

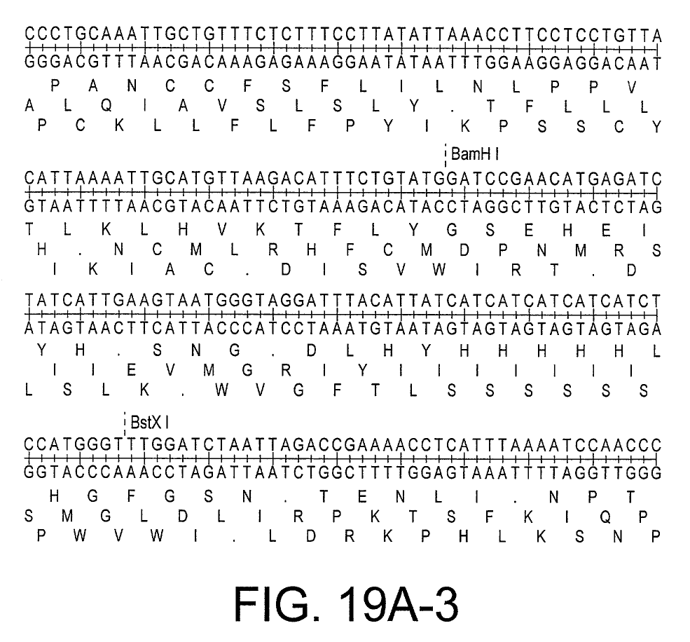

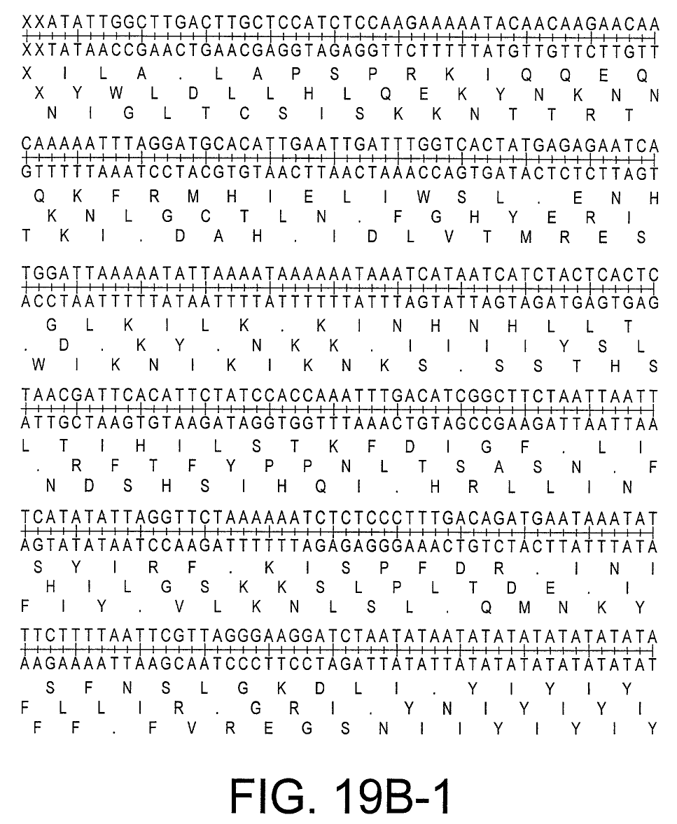

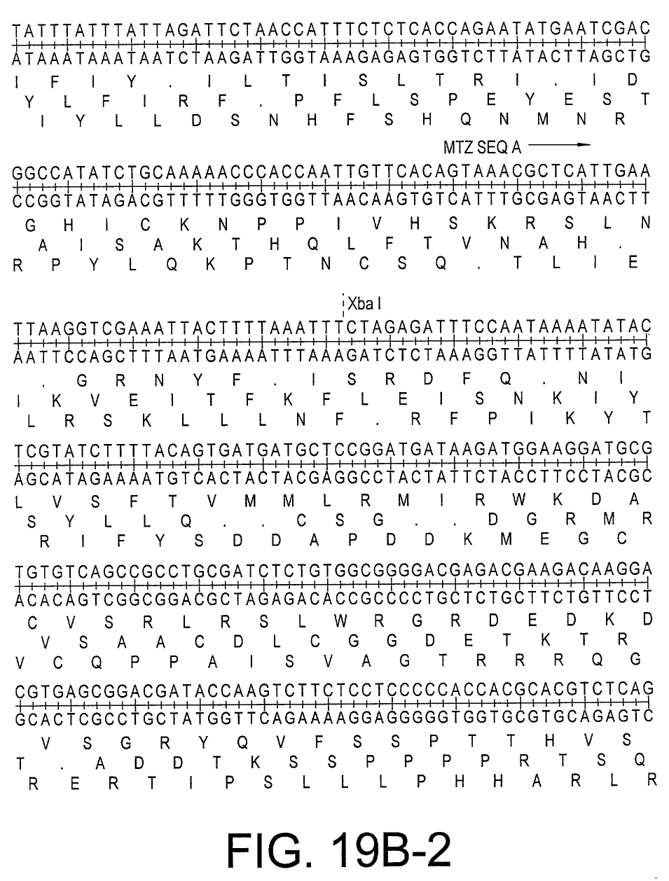

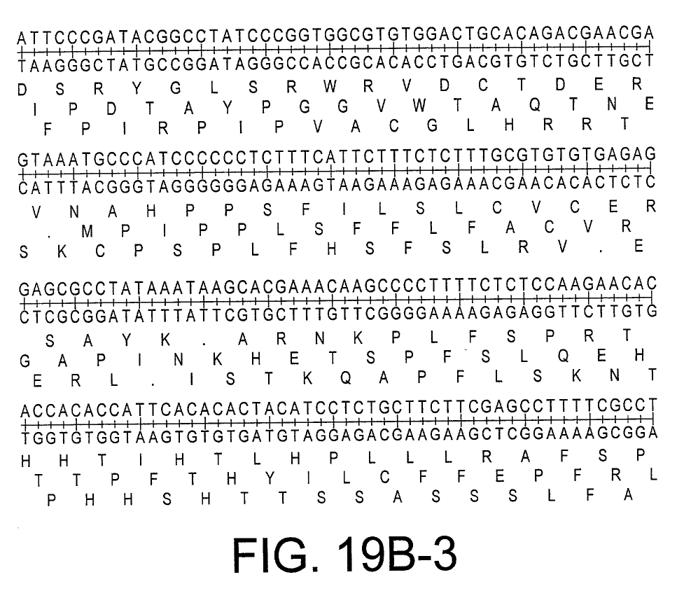

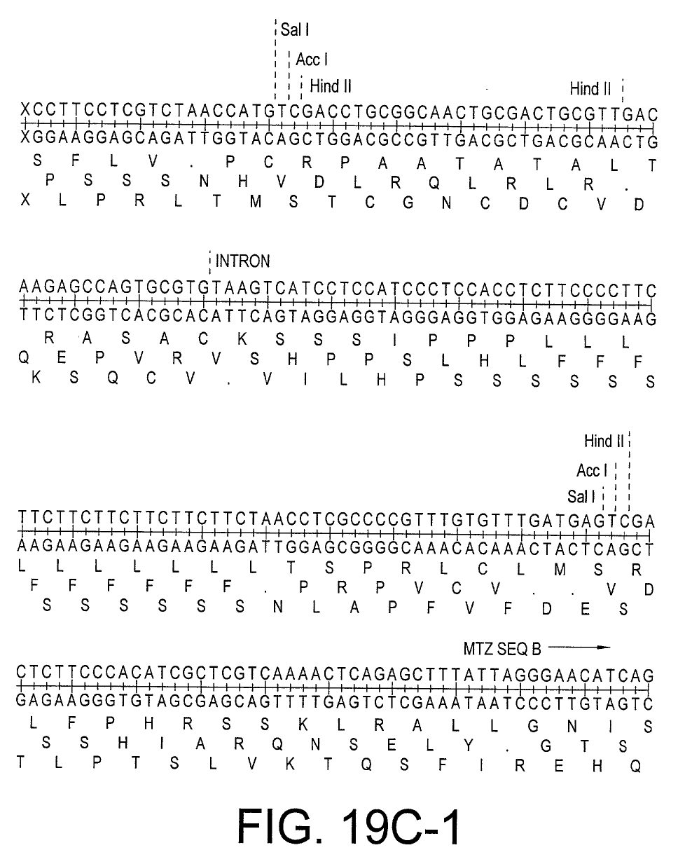

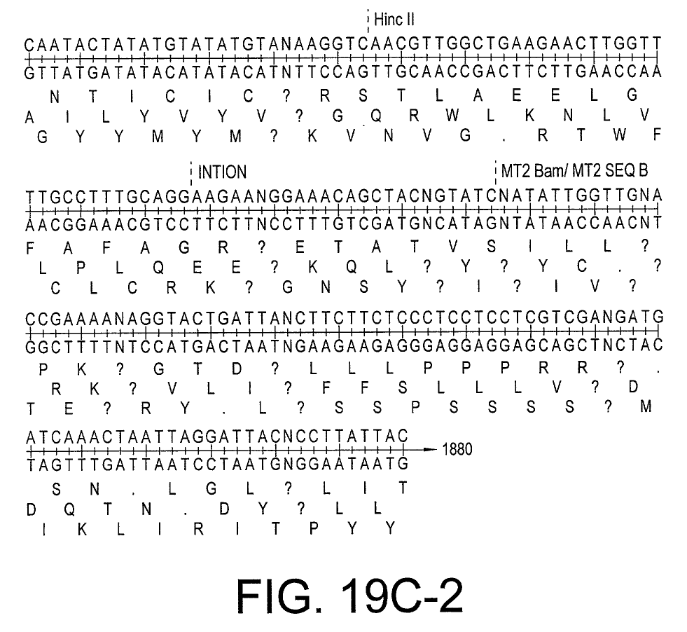

5. A DNA molecule according to claim 1, wherein said DNA molecule encodes a protein having an amino acid sequence selected from the group consisting of SEQ ID NO: 4, SEQ ID NO: 5, SEQ ID NO: 6, the DNA sequence shown in FIG. 16, the DNA sequence shown in FIG. 17, the DNA sequence shown in FIG. 18, and the DNA sequence shown in FIG. 19.

6. A chimeric gene comprising a DNA molecule which is differentially expressed during banana fruit development operably linked to a heterologous promoter.

7. A replicable expression vector comprising the chimeric gene of claim 6.

8. A plant genome, comprising the chimeric gene of claim 6.

9. A plant cell, comprising the chimeric gene of claim 6.

10. A plant comprising the chimeric gene of claim 6, wherein said chimeric gene is stably integrated into the plant genome.

11. An isolated and purified banana protein which is differentially produced in developing banana fruit.

12. A protein according to claim 11, wherein said protein is a selected from the group consisting of a starch synthase, a granule-bound starch synthase, a chitinase, an endochitinase, a beta-1,3 glucanase, a thaumatin-like protein, an ascorbate peroxidase, a metallothionein, a lectin, and another senescence-related protein.

13. A protein according to claim 11, wherein said protein is encoded by a DNA molecule selected from the group consisting of clones pBAN 3-33, pBAN 3-18, pBAN 3-30, pBAN 3-24, pBAN 1-3, pBAN 3-28, pBAN 3-25, pBAN 3-6, pBAN 3-23, pBAN 3-32, and pBAN 3-46.

14. A protein according to claim 11, wherein said protein has an amino acid sequence selected from the group consisting of SEQ ID NO: 4, SEQ ID NO: 5, SEQ ID NO: 6, the amino acid sequence shown in FIG. 16, the amino acid sequence shown in FIG. 17, the amino acid sequence shown in FIG. 18, and the amino acid sequence shown in FIG. 19.

15. A composition comprising the protein of claim 11 and a carrier therefor.

16. A plant cell comprising the protein of claim 11.

17. An isolated and purified banana DNA regulatory element which is 5′ or 3′ to a gene which is differentially expressed during fruit development.

18. A regulatory element according to claim 17, wherein said regulatory element is activated by an ethylene signal.

19. A regulatory element according to claim 18, wherein the ethylene signal is produced by developing fruit.

20. A regulatory element according to claim 18, wherein the ethylene signal is produced by exogenous ethylene gas.

21. A chimeric gene comprising a banana DNA regulatory element operably linked to a heterologous DNA molecule, wherein said regulatory element is naturally found, or is derived from a sequence naturally found, 5′ or 3′ to a gene which is differentially expressed during fruit development.

22. A plant genome comprising a chimeric gene according to claim 21.

23. A plant cell transformed with a chimeric gene according to claim 21.

24. A plant comprising a chimeric gene according to claim 21, wherein said chimeric gene is stably integrated into the plant genome.

25. A method for expression of heterologous protein in fruit comprising transforming fruiting plants with a chimeric gene according to claim 21, exposing said fruit to an plant development signal, and harvesting fruit containing said heterologous protein.

26. The method of claim 25, wherein the plant development signal is ethylene gas produced by ripening fruit.

27. The method of claim 25, wherein the plant development signal is exogenous ethylene gas.

28. The method of claim 25, further comprising the step of isolating the heterologous proteins from the harvested fruit.

29. The method of claim 25, wherein the heterologous protein is a therapeutic protein.

30. A fruit produced by the method of claim 25.

31. The fruit of claim 30, wherein said fruit is a banana.

32. A protein produced by the method of claim 25.

33. A protein produced by the method of claim 28.

34. A plant comprising a chimeric gene according to claim 24, wherein said plant is a monocot.

35. A plant comprising a chimeric gene according to claim 34, wherein said monocot is a banana plant.

36. A plant comprising a chimeric gene according to claim 24, wherein said plant is a dicot.

37. A plant comprising a chimeric gene according to claim 36, wherein said dicot is a tomato plant.

38. The fruit of claim 30, wherein said fruit is a tomato.

39. A regulatory element according to claim 17, wherein said regulatory element is a 5′ upstream promoter region of the p31 gene.

40. A regulatory element according to claim 39, wherein said regulatory element has the nucleotide sequence of SEQ ID NO: 44 or is a fragment thereof.

41. A regulatory element according to claim 39, wherein said 5′ upstream region of the p31 gene has the nucleotide sequence of SEQ ID NO: 45 or is a fragment thereof.

42. A chimeric gene according to claim 21, wherein said regulatory element is a 5′ upstream promoter region of the p31 gene.

43. A chimeric gene according to claim 21, wherein said regulatory element has the nucleotide sequence of SEQ ID NO: 44 or is a fragment thereof.

44. A chimeric gene according to claim 21, wherein said regulatory element has the nucleotide sequence of SEQ ID NO: 45 or is a fragment thereof.
